(19) United States
(12) Patent Application Publication
(10) Pub. No.: US 2025/0295729 A1
(43) Pub. Date: Sep. 25, 2025

(54) NOVEL THERAPEUTIC METHODS OF USING PEPTIDOGLYCAN MUROPEPTIDES TO PROMOTE ATP SYNTHASE ACTIVITY AND MITOCHONDRIAL HOMEOSTASIS AND DEVELOPMENT

(71) Applicant: The Regents of the University of Colorado, a body corporate, Denver, CO (US)

(72) Inventors: Min Han, Boulder, CO (US); Dong Tian, Boulder, CO (US); Aileen K Sewell, Longmont, CO (US); Mingxue Cui, Boulder, CO (US)

(21) Appl. No.: 18/682,389

(22) PCT Filed: Aug. 8, 2022
(86) PCT No.: PCT/US2022/074654

§ 371 Date: Feb. 8, 2024

Related U.S. Application Data
(60) Provisional application No. 63/231,350, filed on Aug. 10, 2021.

(51) Int. Cl. A61K 38/14; A61P 25/00; A61P 25/28

(52) U.S. Cl. CPC A61K 38/14; A61P 25/00; A61P 25/28

(57) ABSTRACT

The present inventive technology is directed the novel therapeutic application of muropeptides as class of novel ATP synthase agonists. In particular, the invention includes systems, methods and compositions for the use of muropeptides as novel ATP synthase agonists, and their use as a therapeutic agents to treat diseases and conditions that involve abnormal ATP synthase and mitochondrial activities.

### CROSS-REFERENCE TO RELATED APPLICATIONS

This International PCT Application claims the benefit of and priority to U.S. Provisional Application No. 63/231,350, filed Aug. 10, 2021, which is incorporated herein by reference in its entirety.

### STATEMENT OF GOVERNMENT INTEREST

This invention was made with government support under grant number 5R01GM047869 awarded by the National Institutes of Health. The government has certain rights in the invention.

### TECHNICAL FIELD

The present inventive technology include novel ATP synthase agonists, and their use as a therapeutic agents to treat diseases and conditions that involve abnormal ATP synthase and mitochondrial activities.

### BACKGROUND

A growing body of studies have revealed the enormously complexity and diversity of gut microbiota and their profound impact on a broad range of physiological functions in host animals. Based on the “symbiosis” concept that describes the interdependent relationship between commensal microbes and host, animals have evolved various mechanisms to beneficially use many microbial metabolites to enhance their reproductive fitness. However, our understanding of these beneficial roles and the underlying mechanisms for individual microbial metabolites are still limited largely due to the challenging nature of studies using animal models. Therefore, developing effective animal models and new approaches to explore the role of individual microbial metabolites are highly desirable.

Peptidoglycan (PG) is a crucial and unique component of bacterial cell walls in both of Gram-positive and Gram-negative species. The PG structure is made of a glycan backbone of repeating N-acetylglucosamine (NAG) and β-(1-4)-Nacetylmuramic acid (NAM) that are cross-linked by short peptides. During bacterial proliferation, up to 50% of PG is degraded by a turnover process (PG hydrolysis) that generates PG fragments with diverse structures known as muropeptides (See general description of muropeptides in Irazoki et al. 2019, which is incorporated by reference herein). While the majority of the muropeptides from PG hydrolysis or autolysis are reused by a PG recycling pathway in bacteria, muropeptides are also released to the environment, which is the intestine for commensal bacteria. Muropeptides are known to interact with host proteins for roles in bacterial pathogenicity and host innate immune responses (Humann and Lenz, 2009; Irazoki et al., 2019). Previous studies have shown that eukaryotes detect intact PG or its fragments by peptidoglycan recognition proteins (PGRPs) or Nod-like receptors (NLRs). PG has been detected within animal cells, and high PG levels in animal cells were reported to correlate with most of the clinical manifestations of bacterial infections, such as fever, inflammation, septic shock, leukocytosis and arthritis. However, some recent studies also suggested roles for PG molecules beyond pathogenicity and immune responses in animal hosts. The specific PG molecules involved in the beneficial impact on animal physiology.

As described herein, the inventive technology is directed the novel therapeutic application of muropeptides as class of compounds to treat a disease or condition. In particular, the inventive technology includes the discovery of an unexpected beneficial role of muropeptides in regulating host mitochondria (Mt) homeostasis, development and food behavior, as well as the underlying mechanism.

### SUMMARY OF THE INVENTION

One aspect of the inventive technology described herein includes the novel therapeutic application of bacterial PG fragments in supporting mitochondrial homeostasis, animal development and behavior. These beneficial PG fragments include one or more mono-, or preferably disaccharide muropeptides, or mixtures of the same containing a short amino acid chain (hereinafter generally referred to as “muropeptides” or “therapeutic muropeptides.”) The therapeutic muropeptide of the invention may further be derived from a PG that is not associated with a lipoprotein.

In certain aspects, the invention may include one or more isolated therapeutic muropeptides, which may include a single form of an therapeutic muropeptide, for example a therapeutic muropeptide comprising a 5′NAG-NAM disaccharide muropeptides with an amino acid peptide attached to NAM. In other preferred the invention may include one or more isolated therapeutic muropeptides, which may include a complex mixture of therapeutic muropeptide that each have distinct compositions. For example, a complex mixture may include therapeutic muropeptides having mono-, di-, or tri-saccharide structures with vary compositions and positions of associated peptides. In further embodiment, the invention may include one or more isolated muropeptides in a complex mixture having therapeutic muropeptide as well as muropeptides that do not have a therapeutic effect. In this example, the complex mixture may include a bacterial extract, or fraction of a bacterial extract containing a complex mixture of different muropeptides compositions. In each of the foregoing embodiment, the complex mixtures of muropeptides and/or therapeutic muropeptides may be derived from a PG that is not associated with a lipoprotein.

In another aspect, a therapeutic muropeptide of the invention may include a 5′ NAG-NAM disaccharide muropeptide with an amino acid peptide coupled to the NAM. In one preferred aspect, the therapeutic muropeptide of the invention is not associated with a lipoprotein. In a preferred aspect, a therapeutic muropeptide of the invention may include a 5′ NAG-NAM disaccharide muropeptide with an amino acid peptide coupled to the NAM, and wherein the therapeutic muropeptide is not associated with a lipoprotein, or wherein the PG molecules form which it was derived was not associated with a lipoprotein.

In another aspect, the invention may include methods of treating a mitochondrial disease or condition. In a preferred aspect, the method may include administering a therapeutically effective amount of isolated therapeutic muropeptide combined with a pharmaceutically acceptable carrier to a subject in need thereof. In a preferred aspect, a therapeutic muropeptide of the invention may include a 5′ NAG-NAM disaccharide muropeptide with an amino acid peptide coupled to the NAM, and wherein the therapeutic muropeptide is not associated with a lipoprotein, or wherein the PG molecules form which it was derived was not associated with a lipoprotein.

In another aspect, the invention may include a pharmaceutical composition for the treatment of a mitochondrial disease. In a preferred aspect, the pharmaceutical composition of the invention may include a therapeutically effective amount of isolated therapeutic muropeptide combined with a pharmaceutically acceptable carrier which may be administered to a subject in need thereof. In a preferred aspect, a therapeutic muropeptide of the invention may include a 5′ NAG-NAM disaccharide muropeptide with an amino acid peptide coupled to the NAM, and wherein the therapeutic muropeptide is not associated with a lipoprotein, or wherein the PG molecules form which it was derived was not associated with a lipoprotein.

In another aspect, the invention may include systems and methods of producing a therapeutic muropeptide comprising: establishing a quantity of peptidoglycan (PG); removing any lipoproteins associated with the PG; treating the PG with a lysozyme generating a quantity of therapeutic muropeptides; and isolating the therapeutic muropeptides. In another preferred embodiment, the therapeutic muropeptides of the invention may be combined with a pharmaceutically acceptable carrier. In a preferred aspect, a therapeutic muropeptide of the invention may include a 5′ NAG-NAM disaccharide muropeptide with an amino acid peptide coupled to the NAM, and wherein the therapeutic muropeptide is not associated with a lipoprotein, or wherein the PG molecules form which it was derived was not associated with a lipoprotein.

In another aspect, the invention may include a novel ATP synthase agonist comprising an isolated therapeutic muropeptide. In a preferred aspect, a therapeutic muropeptide of the invention may include a 5′ NAG-NAM disaccharide muropeptide with an amino acid peptide coupled to the NAM, and wherein the therapeutic muropeptide is not associated with a lipoprotein, or wherein the PG molecules form which it was derived was not associated with a lipoprotein.

In another aspect, the invention may include a novel method of stabilizing an ATP synthase complex comprising contacting one or more subunits of the ATP synthase complex with an isolated therapeutic muropeptide. In a preferred aspect, a therapeutic muropeptide of the invention may include a 5′ NAG-NAM disaccharide muropeptide with an amino acid peptide coupled to the NAM, and wherein the therapeutic muropeptide is not associated with a lipoprotein, or wherein the PG molecules form which it was derived was not associated with a lipoprotein.

In another aspect, the invention may include a novel method of increasing ATP production in an assay. In one embodiment, this may include contacting one or more subunits of said ATP synthase complex an in vitro or in vivo with an isolated therapeutic muropeptide. In a preferred aspect, a therapeutic muropeptide of the invention may include a 5′ NAG-NAM disaccharide muropeptide with an amino acid peptide coupled to the NAM, and wherein the therapeutic muropeptide is not associated with a lipoprotein, or wherein the PG molecules form which it was derived was not associated with a lipoprotein.

In another aspect, the invention may include a novel assay having increased ATP production. In one embodiment, this may include an in vitro or in vivo assay that requires ATP synthesis, which includes a quantity of isolated therapeutic muropeptide. In a preferred aspect, a therapeutic muropeptide of the invention may include a 5′ NAG-NAM disaccharide muropeptide with an amino acid peptide coupled to the NAM, and wherein the therapeutic muropeptide is not associated with a lipoprotein, or wherein the PG molecules form which it was derived was not associated with a lipoprotein.

Additional aspect of the invention may include one or more of the following embodiments. The present application refers to various journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### BRIEF DESCRIPTION OF THE FIGURES

FIGS. 1A-F. Peptidoglycan (PG) from E. coli is required for the development and food behavior in C. elegans. (A) List of 4 E. coli PG metabolic genes identified in a screen of the Keio mutant collection (E. coli K-12). DD-Pase, DD-carboxypeptidase and DD-endopeptidase; LD-TPase, LD-transpeptidase. (B) Bar graph showing worms fed each of 4 PG mutants displayed growth delay as indicated by percentage of worms at the indicated developmental stages. Larval stages were visually determined based on vulval development. n=53-140. (C) Worms fed with each of 4 PG mutants exhibited food avoidance behavior. Data are presented as the percentage of worms found On or Off the bacterial lawn. 100-200 animals/assay. Data are represented as mean±SEM from three replicates. (D-F) Bar graphs and representative microscope images showing that both the growth delay (D, n=49-78) and food avoidance (E and F, 100-200 animals/assay, mean±SEM from three replicates) phenotypes were suppressed by adding heat-killed wild type K-12 bacteria (HK-WT) or isolated PG.

FIGS. 2A-H. Absence of beneficial PG metabolites induces Mt stress in C. elegans. (A-D) GFP fluorescence images and bar graphs showing that feeding each of the 4 PG mutant E. coli induced Mt stress as indicated by Mt stress/UPR^{mt} reporters (Phsp-6::gfp and Phsp-60::gfp), and nuclear accumulation of DVE-1::GFP in the intestine. n=31-68 in (B), n=31-32 in (C), and n=39-40 in (D). Error bars: mean±SD. Scale bar, 100 μm for the top and middle panels in (A), 20 μm for the bottom panels in (A). (E and F) GFP fluorescence images and bar graph showing PG mutant diet did not trigger ER stress (Phsp-4::gfp, n=27-37, mean±SD) or cytoplasmic stress (Phsp-16.2::gfp, n=32-36, mean±SD). Tunicamycin (n=29) and heat shock (n=32) were used as positive controls for ER stress and cytoplasmic stress, respectively. Scale bar in (E), 100 μm. (G) GFP images and bar graph showing PG isolated from wild type E. coli suppressed Mt stress (Phsp-6::GFP) induced by feeding each of the 4 PG mutant E. coli (n=30-37, mean±SD). Scale bar, 100 μm. (H) Feeding erfK mutant E. coli upregulated the level of SOD-3::GFP and the increase was suppressed by adding PG from wild type E. coli or NAC. (n=29-30, mean±SD). (I-K), Bar graphs and representative microscope images showing that NAC supplementation suppressed growth delay (I, n=52-61), Mt stress (J, n=30-31) and food avoidance behavior (K, 100-200 animals/assay, mean±SEM from 3 replicates). Scale bar in (J), 100 μm.*** p<0.001, ns: not significant.

FIGS. 3A-G. Beneficial muropeptides contain disaccharides and short peptides. (A) Cartoon diagram of a large PG molecule and the cleavage points of 3 enzymes indicated by black arrows. NagZ: Glucosaminidase; AmiD: Amidase; NAG: 3′ N-acetylglucosamine; NAM: 5′ N-acetylmuramic acid. (B-D) Bar graphs and microscope images showing the effects of treating isolated PG with the indicated enzymes. ProtesaseK (ProK), AmiD or NagZ treated PG failed to suppress the growth delay (B, n=50-59), Mt stress (C, n=30-31, mean±SD) or food avoidance (D, 100-200 animals/assay, mean±SEM from 3 replicates) phenotypes in animals fed the erfK mutant E. coli. PG and lysozyme (lyso) treated PG suppressed all three phenotypes. (E-G) Muropeptides isolated from lysozyme-treated PG suppressed growth delay (E, n=30-65), Mt stress (F, n=30-32, mean±SD) and food avoidance (G, 100-200 animals/assay, mean±SEM from 3 replicates). Scale bar in (C) and (F), 100 μm.

FIGS. 4A-D. Muropeptides enter and accumulate in host intestinal mitochondria. (A) FITC labeled PG (FITC-PG) suppressed the growth defect of worms fed erfK mutant E. coli (n=64-80). (B) Representative images and quantitative data showing FITC-muropeptide accumulation in intestinal cells of C. elegans that were also fed with MitoTracker as a control. Animals were fixed to remove intestinal auto-fluorescence. n=17-18, mean±SD. (C) Representative images and quantitative data showing co-localization of FITC-muropeptides and MitoTracker in a subset of isolated Mt (FITC-positive Mt in upper panel). Quantification showing that 34% of total isolated Mt are FITC-positive, 66% are FITC-negative. FITC-positive Mt may be derived from intestinal cells while FITC-negative Mt may represent Mt from non-intestinal cells. Mean±SEM, n=336 from 3 replicates. (D) Data from an NF-κB activity assay that indicates the interaction between muropeptides from C. elegans Mt lysates and NOD-1 in mammalian cells. Mt lysate from worms fed wild-type E. coli induced higher NF-κB activity than the lysate from worms fed erfK mutant E. coli. Mean±SEM from 3 replicates. *** p<0.001, ** p<0.01.

FIG. 5A-N. Muropeptides bind to ATP synthase in the host for the beneficial function. (A-D) RNAi knockdown of 5 PG interactor candidates triggered Mt stress (A and B, n=33-37, mean±SD), growth delay (C, n=49-60) and food avoidance behavior (D, 100-200 animals/assay, mean±SEM from 3 replicates). (Representative images for D are shown in FIG. 15B). Scale bar in (A) is 100 μm. (E) Interaction between PG and ATP-1 detected by in vivo pull-down assays. ATP-1 from worm lysates was bound to PG and the binding increased in a concentration dependent manner. (F) Protease-K treatment (to which short peptides on PG are sensitive), but not trypsin treatment (to which the short amino acid peptides on PG are insensitive), eliminated the binding of PG to ATP-1. (G) Trypsin treatment in Step 4 of the PG purification procedure (FIG. 13A), which is expected to remove lipoproteins attached to PG, is required for PG to pull down ATP-1 from worm lysates. (H) GFP images and bar graph showing that the level of FITC-muropeptides in intestinal cells was decreased in atp-4 RNAi knockdown animals. Animals were fixed to remove intestinal autofluorescence. n=31, mean±SEM. (I-K) Representative images and bar graphs showing the dependence of the beneficial role of PG on ATP synthase. In atp-4 RNAi knockdown animals, addition of PG failed to suppress Mt stress (I, n=30-34, mean±SD), growth delay (J, n=53-62) and food avoidance behavior (K, 100-200 animals/assay, mean±SEM from 3 replicates) when feeding with erfK mutant E. coli. atp-4i=atp-4 RNAi treatment. Scale bar in (H), 5 μm; in (I), 100 μm. (L-N) Bar graphs showing that growth delay (L, n=54-62), Mt stress (M, n=30-34, mean±SD) and food avoidance behavior (N, 100-200 animals/assay, mean±SEM from 3 replicates) in worms fed erfK mutant E. coli were partially suppressed by the mai-2(−) mutation, mai-2 (xm19). *** p<0.001.

FIG. 6. PG muropeptides naturally accumulate in the mitochondria of mouse small intestinal cells. Data from a NF-κB activity assay that indicates the interaction between muropeptides from Mt lysates of mouse primary intestinal epithelial cells and NOD-1 receptor. Mt lysate from mouse intestinal epithelial cells induced dramatically higher NF-κB activity than the lysate from MODE-K cultured intestinal epithelial cells. Mean±SEM from 3-4 replicates.

FIG. 7A-K. Muropeptides stabilize ATP synthase and enhance Mt oxidative phosphorylation in cultured human intestinal epithelial cells. (A) Interaction between PG and human ATP synthase α subunit (ATP5A1) detected by in vivo pull-down assays. ATP5A1 from lysates of cultured human intestinal epithelial (caco-2) cells bound to PG and the binding increased with increased caco-2 cell lysate input. (B) Protease-K treatment, but not trypsin treatment, eliminated the binding of PG to ATP5A1. (C-D) PG specifically bound to a, d and F6 subunits, but not β subunit of mammalian (C) or worm (D) ATP synthase. (E) Blue-Native PAGE (BN-PAGE) analysis showing that ATP synthase Complex V in mitochondrial lysates from caco-2 cells was increased by adding muropeptides. Immunoblotting performed with anti-ATP5A1 antibody. SDS-PAGE electrophoresis and subsequent immunoblotting of ATP5A1 was used as the loading control. +: 5 ng/ml; ++: 500 ng/ml; +++: 500 μg/ml muropeptides. (F) Complex V activity increased with increased muropeptide concentration. Data are averaged from 3 replicates. (G) Data from a Mt oxidative respiration analysis of caco-2 cells showing the increase in oxygen consumption rate (OCR) by supplementation with muropeptides (800 μg/ml). Oligomycin (2.5 μM), an ATP synthase inhibitor, was added to evaluate the ATP-linked increase in OCR. FCCP (1.0 μM), an uncoupling agent that raises OCR to maximal value by disrupting the Mt proton gradient and membrane potential, was added to measure maximal OCR. Antimycin and rotenone (1.25 M), two inhibitors of the Mt respiration chain, were added to evaluate non-Mt OCR. The “mock” sample was from the same cells supplemented with lysozyme solution without PG. Mean±SEM from 3 replicates. (H) Bar graphs of the OCR quantification show that the majority of the increase in OCR in caco-2 cells by muropeptide (800 μg/ml) supplementation was due to increase in ATP-linked or Mt-related OCR. Basal OCR: last OCR measurement before oligomycin injection. ATP-linked OCR: basal OCR minus minimal OCR value after oligomycin injection. Maximal OCR: maximum OCR measurement after FCCP injection. Mean±SEM from 3 replicates. (I) Bar graph showing significant increase of ATP level in caco-2 cells supplemented with muropeptides (800 μg/ml). Mean±SEM from 3 replicates. (J) Microscope images and bar graphs showing that addition of muropeptides (800 μg/ml) to caco-2 cells increased Mt membrane potential (Δψₘ) as indicated by MitoTracker-Red CMXRos (red fluorescence). n=108-126, mean±SEM. Scale bar=20 μm. (K) Microscope images and bar graph showing that addition of muropeptides (800 μg/ml) to caco-2 cells suppressed ROS production as indicated by H₂DCFDA (green fluorescence). n=49-57, mean±SEM. Scale bar=20 μm. *** p<0.001, * p<0.05, ns: not significant.

FIGS. 8A-G. Muropeptides significantly recover the mitochondrial function in m.8993T>G mutant fibroblast cells. (A) Analysis of Mt oxidative respiration of m. 8993T>G fibroblast cells (derived from a Leigh Syndrome patient; obtained from Coriell Inst), showing that muropeptide supplementation drastically increased oxygen consumption rate (OCR) that is impaired in the mutant cells. The “mock” treatment was lysozyme solution without PG. Mean±SEM from 3 replicates. (B-D) Bar graphs of the OCR quantification showing that the basal OCR, ATP-linked OCR and maximal OCR in mutant fibroblast cells were increased by muropeptides supplementation. Mean±SEM from 3 replicates. (E) Bar graph showing significant increase of ATP level in m.8993T>G mutant cells supplemented with muropeptides (500 μg/ml). Mean±SEM from 3 replicates. (F) Microscope images and bar graphs showing that addition of muropeptides (50 μg/ml) to m.8993T>G mutant cells increased Mt membrane potential (AW/m) as indicated by MitoTracker-Red CMXRos (red fluorescence). n=70-71, mean±SEM. Scale bar in (H), 20 μm. (G) Representative images and bar graph showing that muropeptides (50 ug/ml) supplementation reduced ROS levels in m.8993T>G mutant cells as indicated by H2DCFDA dye staining. n=64-68, mean±SEM. Scale bar in (G), 20 μm. *** p<0.001, * p<0.05, ns: not significant.

FIG. 9A-D. Muropeptides promote fitness and survival of m.8993T>G mutant fibroblast cells from Leigh Syndrome patients. (A) Puromycin immunoprecipitates of m.8993T>G mutant cells from Leigh Syndrome patients showed increased levels of creatine kinase protein, which is one of the pathological features identified in the patient, compared to wild type cells (left panel). Muropeptide supplementation significantly decreased creatine kinase protein (right panel), n=3 replicates. (B) m.8993T>G mutant cells produced more lactate, which is the end-product of glycolysis, compared to wild type cells. Muropeptide supplementation significantly decreased lactate levels. Lactate levels were measured from media after 24 h culture and muropeptide treatment. Mean±SEM from 3 replicates. (C) m.8993T>G mutant cells generated more ammonia, which was suppressed by muropeptide (50 μg/ml) supplementation. Mean±SEM from 3 replicates. (D) Cell viability in galactose medium. m.8993T>G mutant cells exhibited low viability in galactose medium, which was partially rescued by muropeptide (50 ug/ml) supplementation. Data was normalized to that under normal glucose condition. Mean±SEM from 3 replicates. *** p<0.001, * p<0.05.

FIGS. 10A-B. A model for the beneficial role of bacterial muropeptides in host animals (A) E. coli-produced disaccharide muropeptides act as an ATP synthase agonist by entering host Mt and binding to ATP synthase to promote its activity. The increased ATP synthesis promotes Mt membrane potential (Δψₘ) and oxygen consumption rate (OCR), as observed in mammalian intestinal epithelial cells (IECs), as well as host development and food dwelling behavior in C. elegans. (B) Lack of muropeptides (PG mutant E. coli feeding or germ-free condition) inhibits ATP synthase activity and increases ROS production. The decreased ATP synthesis trigger Mt stress, growth delay and food avoidance behavior in C. elegans. Given that bacterial muropeptides are known to enter cells in mammals and the structure of ATP synthase is exceedingly conserved, such a beneficial role of muropeptides is likely conserved in mammals.

FIGS. 11A-B. Additional data on food avoidance behavior and growth delay, related to FIG. 1. (A) Representative images showed feeding with PG mutant E. coli trigger a food avoidance behavior. Statistic data in FIG. 1C. (B) Mixing two of the PG mutant strains induced growth delay at similar levels as feeding with single PG mutant E. coli strains. n=52-67.

FIGS. 12A-G. The host defects induced by feeding PG mutants are not likely due to iron deficiency or innate immune responses induction, related to FIG. 2. (A) Feeding the mixture of two PG mutants to animals induced Mt stress at similar levels as single mutants. n=30-32, mean±SD. Scale bar: 100 μm. *** p<0.001, ns: not significant. (B-D) Iron supplementations did not suppress Mt stress (B and C, n=26-31, mean±SD) or growth delay (D, n=50-62). Scale bar in (B), 100 μm. (E) PG mutants had no effect on the expression of p38-dependent reporter Pirg-5::gfp. n=50-77, mean±SD. Scale bar: 20 μm. (F) PG mutants suppressed the expression of p38-independent reporter Pirg-1::gfp. n=31-37, mean #SD. Scale bar: 20 μm. (G) ROS staining by the chemical (H2DCFDA) also confirmed that feeding erfK mutant E. coli upregulated the ROS level and the increased ROS was suppressed by adding PG from wild type E. coli or NAC. (n=15, mean±SD).

FIGS. 13A-D. Effects of supplementation of PG products from mutant E. coli to animals feeding with live PG mutants, related to FIG. 3 (A) Diagram of the PG isolation procedure. (B-D) Phsp-6::gfp fluorescence images and bar graphs showing the effects of feeding animals with mixture of the product of indicated PG purification step from PG mutants and the corresponding live PG mutant E. coli on Mt stress (Phsp-6::gfp expression) (B and C) or postembryonic growth (D). Step 1-4 products from dacB or ycbB mutants failed to inhibit Mt stress (B and C, n=29-37, mean±SD) or growth delay (D, n=38-75), confirming the lack of effective muropeptides in the PG molecules from strains with mutations in these two enzymes that are expected to act in PG hydrolysis (Typas et al., 2011). Product from step 4, but not step 1-3, from erfK or ygeR suppressed Mt stress (B and C, n=27-32, mean±SD) or growth delay (D, n=40-71). Since trypsin treatment was used to remove lipoproteins attached on PG, this result may suggest that altered interaction between lipoprotein and PG may prevent the release of functional muropeptides from these two mutant bacteria or isolated PG without trypsin treatment. However, erfK is thought to encode one of several enzymes that facilitate the attachment of lipoprotein to PG (Magnet et al., 2007; Sanders and Pavelka, 2013), which does not seem to be consistent with the result of trypsin treatment of PG from erfK. ygeR is suggested to encode a putative PG hydrolase based on the presence of a LytM (lysostaphin)-Domain, but the exact function is still unknown (Typas et al., 2011; Uehara et al., 2009). Scale bar in (B), 100 μm.

FIG. 14. Additional functional and localization analysis of FITC-PG in C. elegans, related to FIG. 4. Fluorescent and DIC images showing FITC-PG puncta (arrows) in the intestinal lumen of young adult. Scale bars: 20 μm.

FIGS. 15A-E. Additional data on the requirement of ATP synthase for the beneficial functions of PG in host animals, related to FIG. 5

(A) List of 5 candidate genes for mitochondrial proteins identified in a screen for PG interacting proteins by affinity purification, mass-spectrometry and RNAi knockdown analysis. Sequence coverage and scores are data from mass-spectrometry analysis. (B) Representative images showing RNAi knockdown of 5 candidate genes triggered food avoidance behavior (statistic data in FIG. 5D). (C-D) Representative images showing that Mt stress (C) and food avoidance behavior (D) in worms fed erfK mutant E. coli were partially suppressed in the mai-2(−) mutant (statistical data in FIGS. 5M and 5N). (E) RNAi knockdown of mai-1 only did not suppress the growth rate when feeding with erfK mutant E. coli. RNAi knockdown of mai-1 in mai-2 mutant background did not suppress the growth rate further. n=51-58.

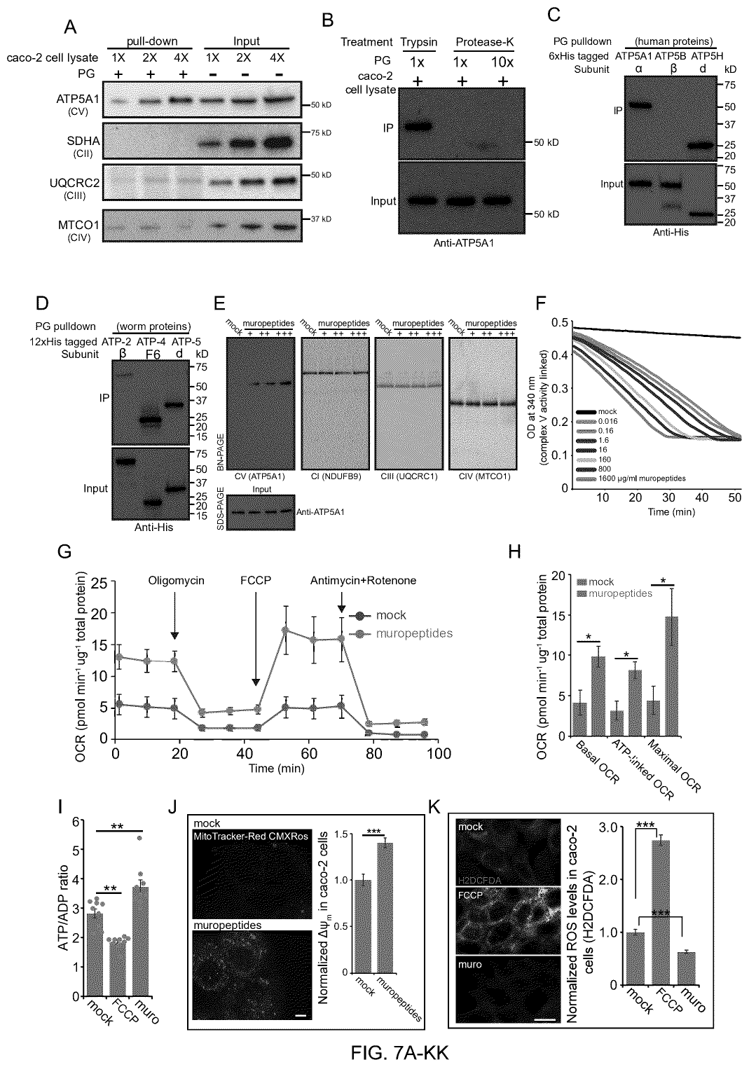

FIG. 16. Muropeptides stabilize the ATP synthase complex from bovine heart mitochondria, related to FIG. 7A-E. (A-B) Representative BN-PAGE electrophoresis of solubilized bovine heart mitochondria. Immunoblotting performed with anti-ATP5A1 antibody. ATP synthase complex formation increased with addition of muropeptides to solubilized mitochondria (A). The quantification from (A) shows that muropeptide supplementation significantly increased the abundance of ATP synthase complex (B). Mean±SEM from 3 replicates. (C-D) Coomassie brilliant blue staining shows purified recombinant mammalian (C) or worm (D) protein bands. (E) Muropeptide supplementation had no effect on ATP5A1 expression in caco-2 cells. Mean±SEM from 3 replicates.

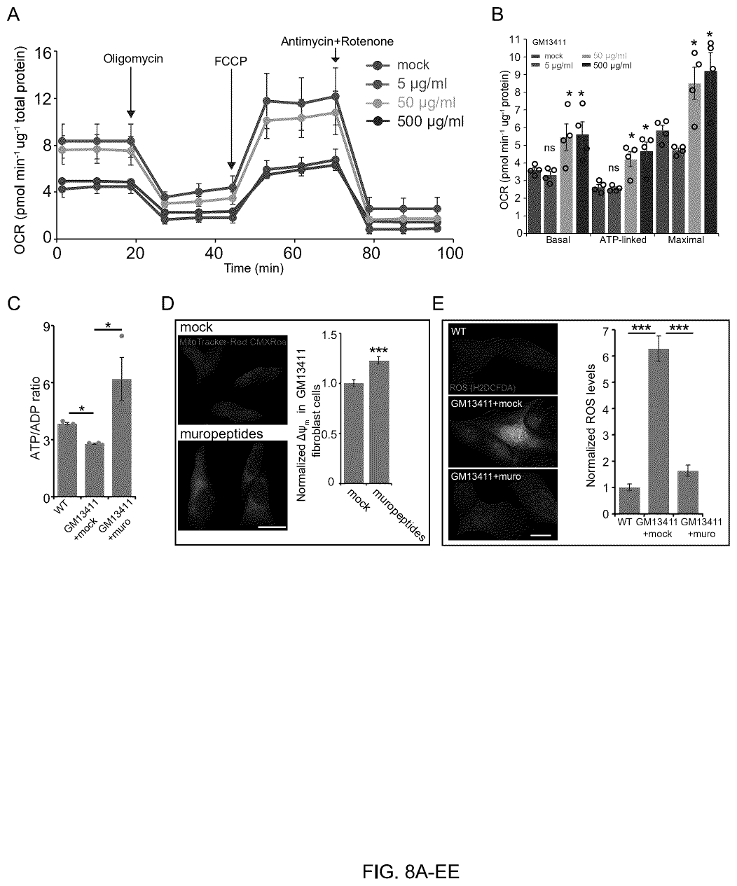

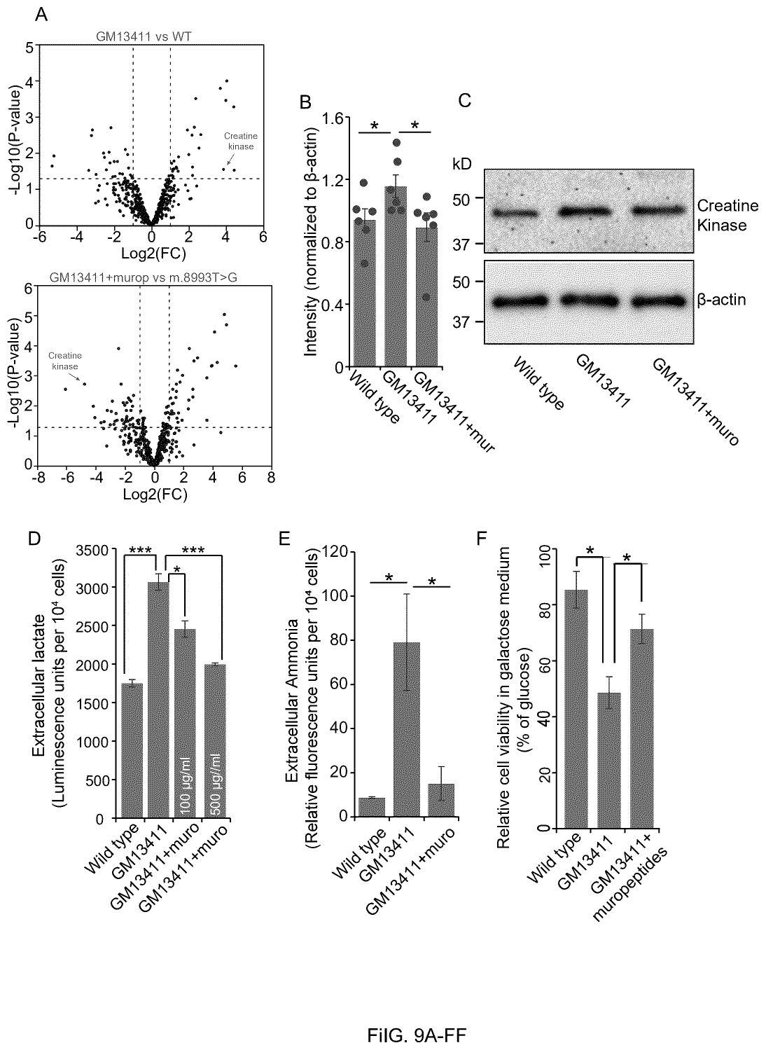

FIGS. 17A-D. mitochondrial function is impaired in m.8993T>G mutant fibroblast cells, related to FIG. 8. (A) Data from a Mt oxidative respiration analysis of m.8993T>G mutant (derived from a Leigh Syndrome patient) and wild type control fibroblast cells showing the decreased oxygen consumption rate (OCR) in the mutant fibroblast cells. Mean±SEM from 3 replicates. (B) Bar graphs of the OCR quantification showing the basal OCR, ATP-linked OCR and maximal OCR in mutant fibroblast cells were decreased in mutant fibroblast cells, compared to wild type control. Mean±SEM from 3 replicates. (C) Bar graph showing significant decrease of ATP level in mutant m.8993T>G fibroblast cells. Mean±SEM from 3 replicates. (D) Representative images and bar graph showing the increased ROS levels in mutant fibroblast cells, compared to wild type control. n=53-56, mean±SEM. Scale bar in (D), 20 μm. *** p<0.001, ** p<0.01, * p<0.05.

FIG. 18A-B. Muropeptides recover mitochondrial structure in m.8993T>G mutant fibroblast cells from Leigh Syndrome patients. (A and B) Representative electron microscopy images (A) and statistical analysis (B) of mitochondrial structures in WT, m.8993T>G mutant, and muropeptide-treated mutant fibroblast cells. While most mitochondria in m.8993T>G fibroblast cells show no cristae (top right panel), muropeptide supplementation recovers cristae structure in mutant fibroblast cells in a concentration dependent manner (bottom panels). “Vesicular-like” indicates that mitochondria have cristae structure, but the cristae morphology is still abnormal. n=68-214 mitochondria from >10 micrographs. Scale bar in (A) is 100 nm. Mean±SEM from 3 replicates. * p<0.05.

FIG. 19A-F. Muropeptides suppress accumulation of vacuoles and endo/autophagosomes in m.8993T>G mutant fibroblast cells, related to FIG. 18.

(A-B) Representative electron microscopy images (A) and quantification (B) show that vacuole accumulation in m.8993T>G mutant cells is suppressed by muropeptide supplementation. Scale bar in (A) 10 μm. (C-D) Representative electron microscopy images (C) and quantification (D) show that endo/autophagosomes are enriched in m.8993T>G mutant cells and muropeptide supplementation decreased this accumulation. (E-F) Western blot and quantitative data showing that the level of HSP60 protein was elevated in m.8993T>G mutant fibroblasts and the level was drastically reduced by muropeptide supplementation. HSP60 is a commonly used marker for mitochondrial stress. (G-H) Western blot and quantitative analysis showing that the levels of LC3-I (cytoplasm) and LC3-II (autophagosome) protein. LC3-II was elevated in m.8993T>G mutant fibroblasts and the level was drastically reduced by muropeptide supplementation. Scale bar in (C) 1 μm. Mean±SEM from 3 replicates. *** p<0.001, ** p<0.01, * p<0.05.

FIG. 20A-J. Analysis of the impact of antibiotic treatment in mice, related to FIG. 21. (A-D) CFU analysis showed that antibiotic oral gavage depleted gut bacteria. N=3-5 mice. (E-H) Representative images and bar graph show that antibiotic treatment (a cocktail containing ampicillin, vancomycin, metronidazole, neomycin and amphotericin B) enlarged the cecum size in male and female mice. Mean±SEM from 4 mice. (I-J) Bar graphs show the effects of antibiotics and PG treatment on spleen size in both male and female mice n=3-4 mice. PG fed to mice is expected to be digested into muropeptides by lysozymes in mice, as we observed in C. elegans. *** p<0.001, ** p<0.01, * p<0.05, ns: not significant.

FIG. 21A-D. PG muropeptides suppress oxidative stress in small intestine of antibiotic-treated mice, related to FIG. 20. (A-B) Representative images and quantification show that antibiotic-induced ROS in small intestinal epithelial cells (IECs) of male and female mice were suppressed by oral gavage of PG. Mean±SEM from 3-4 mice. (C and D) Expression of glycolytic proteins (PKM2, Pyruvate dehydrogenase and GAPDH) was increased in IECs from antibiotic-treated male mice and the increase was suppressed with oral gavage of PG. Mean±SEM from 6 mice. *** p<0.001, ** p<0.01, * p<0.05.

FIG. 22. Muropeptides promote ATP production and survival of cells of two Alzheimer's disease (AD) models. (A) Muropeptide supplementation increased ATP levels in cells of familial type 1 and type 3 AD models (fibroblast cells). Mean±SEM from 3 replicates. (B-C) Muropeptide supplementation increased cell viability in cells of familial type 1 (24h treatment) and type 3 AD (48h treatment) models (fibroblast cells). Mean±SEM from 3 replicates. * p<0.05

FIG. 23. Functional analysis of muropeptide fractions from Bio-6P gel separation. (A) Bio-6P fractionation monitored by UV205 absorption. (B) OCR analysis of the fractions, indicating that fractions 28-33 and 45-46 have biological function (green bars).

FIG. 24. PG muropeptides promote cell survival in Friedreich's Ataxia (FRDA) cell model. Friedreich's Ataxia primary fibroblast cells derived from a human patient were challenged with a glutathione inhibitor, BSO. PG muropeptide supplementation (100 ug/mL) resulted in an increase in cell survival under this stress condition. Error bars represent SEM of 4 technical replicates.

FIG. 25. PG muropeptides promote cell proliferation of fibroblasts from Rett Syndrome, Fragile X Syndrome (FXS), and young and old healthy human patients. (A) Rett Syndrome primary fibroblast cells have lower cell viability (black bar) than age-matched WT fibroblast control cells (gray bar). Total cell count increased with PG muropeptide supplementation (white bar) (500 ug/mL). (B) Fragile X Syndrome primary fibroblast cell proliferation was increased with PG muropeptide supplementation (100 ug/mL). (C) Healthy 1 yr primary fibroblast cells showed increased viability with PG muropeptide supplementation (100 ug/mL). (D) Healthy 1 yr primary fibroblast cells have a higher cell count (gray bar) than healthy 51 yr cells (black bar), consistent with expected defects associated with natural aging. These older cells show increased viability with PG muropeptide supplementation in a dose dependent manner (ug/mL).

### DETAILED DESCRIPTION OF THE INVENTION

In one embodiment, a therapeutic muropeptide of the invention may include a 5′ NAG-NAM disaccharide muropeptide with an amino acid peptide coupled to the NAM. In one preferred embodiment, the therapeutic muropeptide of the invention is not associated with a lipoprotein. In a preferred embodiment, a therapeutic muropeptide of the invention may include a 5′ NAG-NAM disaccharide muropeptide with an amino acid peptide coupled to the NAM, and wherein the therapeutic muropeptide is not associated with a lipoprotein.

In certain embodiments, the invention may include one or more isolated therapeutic muropeptides, which may include a single form of an therapeutic muropeptide, for example a therapeutic muropeptide comprising a 5′NAG-NAM disaccharide muropeptides with an amino acid peptide attached to NAM. In other preferred the invention may include one or more isolated therapeutic muropeptides, which may include a complex mixture of therapeutic muropeptide that each have distinct compositions. For example, a complex mixture may include therapeutic muropeptides having mono-, di-, or tri-saccharide structures with vary compositions and positions of associated peptides. In further embodiment, the invention may include one or more isolated muropeptides in a complex mixture having therapeutic muropeptide as well as muropeptides that do not have a therapeutic effect. In this example, the complex mixture may include a bacterial extract, or fraction of a bacterial extract containing a complex mixture of different muropeptides compositions. In each of the foregoing embodiment, the complex mixtures of muropeptides and/or therapeutic muropeptides may be derived from a PG that is not associated with a lipoprotein.

In other embodiment, a therapeutic muropeptide of the invention may be derived from a bacteria, or a PG molecule. In alternative embodiment, a therapeutic muropeptide of the invention may be synthesized in vitro

Another embodiment of the current invention includes the novel therapeutic application of bacterial PG fragments to suppress mitochondrial oxidative stress in a subject in need thereof. In a preferred embodiment, the therapeutic muropeptides of the invention repress mitochondrial oxidative stress resulting in the reduction/inhibition of reactive oxygen species (ROS) formation. For example, in one embodiment, the invention may include methods and compositions to increasing the Mitochondrial (Mt) oxidative respiration, the method comprising introducing an effective amount of a therapeutic muropeptide, or a complex mixture of therapeutic muropeptides with a protein in the electron transport chain, which may preferably include UCR-1 which is a complex III subunit of electron transport chain.

Another embodiment of the current invention includes the novel therapeutic application of bacterial PG fragments in regulating ATP generation in a cell, and in particular the action of the enzyme ATP synthase. In one preferred embodiment, therapeutic muropeptides interact with ATP synthase and act as an agonist of the ATP synthase activity. In a preferred embodiment, the therapeutic muropeptides of the invention stabilize the ATP synthase complex thereby acting as an ATP synthase agonist, which increases mitochondrial ATP production.

Many human diseases and conditions are tightly associated with mitochondrial dysfunctions, commonly marked by reduction in electron transport chain (ETC) activity and/or ATP production by ATP synthase, as well as increases in mitochondrial ROS production and oxidative stress. These diseases include over 150 so called genetic mitochondrial dysfunction syndromes, where mitochondrial dysfunction was known to be causal to the pathogenesis of the diseases (Dautant et al., 2018). However, accumulative research results have indicated or suggested critical contributions of mitochondrial dysfunction to many other major age-related and developmental disorders including highly prevalent neurodegenerative diseases (e.g., Parkinson's, Alzheimer's, Huntington's, and Amyotrophic Lateral Sclerosis), other neurological disorders (e.g., epilepsy, autism, fibromyalgia, cerebral palsy, and chronic fatigue), Cardiomyopathy, muscular dystrophy, metabolic diseases, and certain types of cancers.

In one embodiment, the invention includes novel therapeutic the novel therapeutic application of bacterial PG fragments for treating a mitochondrial disease or condition. In one preferred embodiment, the invention includes a method of treating a mitochondrial disease or condition including administering a therapeutically effective amount of a therapeutic muropeptide to a subject in need thereof. In another preferred embodiment, the invention includes a method of treating a mitochondrial disease or condition including administering a therapeutically effective amount of a 5′ NAG-NAM disaccharide muropeptide with an amino acid peptide coupled to the NAM to a subject in need thereof, which in another of the invention is not associated with a lipoprotein.

Another embodiment, the invention include novels methods of generating therapeutic muropeptides, which may include an isolated 5′ NAG-NAM disaccharide muropeptide with an amino acid peptide coupled to the NAM to a subject in need thereof, which in another of the invention is not associated with a lipoprotein.

Another embodiment of the current invention includes the novel therapeutic application of bacterial PG fragments in to act as an ATP synthase agonist in an in vitro or in vivo assay. In one preferred embodiment, therapeutic muropeptide of the invention may include a 5′ NAG-NAM disaccharide muropeptide with an amino acid peptide coupled to the NAM, and may further be used in an assay to act as an ATP synthase agonist.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y. and Current Protocols in Molecular Biology (Ausbel et al., eds., John Wiley & Sons, Inc. 2001. As appropriate, procedures involving the use of commercially available kits and reagents are generally carried out in accordance with manufacturer defined protocols and/or parameters unless otherwise noted.

The term “peptidoglycan” describes the major structural polymer in most bacterial cell walls and consists of glycan chains of repeating N-acetylglucosamine and N-acetylmuramic acid residues cross-linked via peptide side chains

The term “muropeptide” as used herein are derived from PG, and include NAG-NAM disaccharides attached to a peptide chain containing 2- to 5 amino acid residues, typically: L-alanine, D-glutamic acid, mDAP/L-Lys, D-alanine, and D-alanine. Muropeptides have diverse cleavage points of PG cleaving enzymes: including, glucosaminidases, amidases, peptidases, and muramidases. The term “therapeutic muropeptide” as used herein describes an isolated muropeptide that produces a therapeutic effect in a subject. In one preferred embodiment, a therapeutic muropeptide produces a therapeutic effect by binding to, and acting as an ATP synthase agonist. In one preferred embodiment, a therapeutic muropeptide produces a therapeutic effect by reducing mitochondrial oxidative stress. In one preferred embodiment, a therapeutic muropeptide 5′NAG-NAM disaccharide muropeptides with an amino acid peptide attached to NAM. In one preferred embodiment, a therapeutic muropeptide 5′NAG-NAM disaccharide muropeptides with an amino acid peptide attached to NAM, which may be derived from PG that was not associated with a PG that is not associated with a lipoprotein, or a 5′NAG-NAM disaccharide muropeptides with an amino acid peptide attached to NAM, which itself is not associated with a lipoprotein.

As used herein, the term “mitochondrial disease” refers to a disease, disorder, or condition in which the function of a subject's mitochondria becomes impaired or dysfunctional, and in particular due to oxidative stress or aberrant ATP production. A “mitochondrial disease” also refers to a disease or condition that can be treated through the administration of a therapeutically effective amount of a PG muropeptide of the invention. A “mitochondrial disease” also refers to a disease or condition that can be treated through increasing the activity of ATP synthase and ETC, as well as suppressing Mt oxidative stress, preferably through the administration of a therapeutically effective amount of a PG muropeptide of the invention

Examples of mitochondrial diseases that may be treated with a compound or method described herein include: Apical hypertrophic cardiomyopathy (AHCM). neuropathy, ataxia, autism, Charcot-Marie-Tooth syndrome (CMT), encephalopathy, epilepsy with brain pseudoatrophy, Episodic Weakness, Hereditary Spastic Paraplegia (HSP), Familiar Bilateral Striatal Necrosis (FBSN), Infantile cardiomyopathy, Leber Hereditary Optic neuropathy (LHON), Left Ventricular Hyper Trabeculation syndrome (LVHT), Maternally inherited Diabetes, Deafness syndrome (MIDD), Maternally inherited Leigh Syndrome (MILS), Mesial Temporal Lobe Epilepsies with Hippocampal Sclerosis (MTLE-HS), Metabolic Syndrome (MS), Motor Neuron Syndrome (MNS), Myopathy, lactic Acidosis, Sideroblastic Anemia (MLASA), Neurogenetic Ataxia Retinis Pigmentosa syndrome (NARP), Periodic paralyzes, Schizophrenia, Spino Cerebellar Ataxia (SCA), Tetralogy of Fallot (ToF), short-chain acyl-coA dehydrogenase deficiency (SCAD), medium-chain acyl-coA dehydrogenase deficiency (MCAD), long-chain acyl-coA dehydrogenase deficiency LCAD), chronic progressive ophthalmoplegia (CPEO), Pearson Syndrome, Barth Syndrome, Alpers Disease, Luft Disease, mitochondrial neurogastrointestinal encephalomyopathy (MNGIE), neuropathy, ataxia, retinal pigmentosa (NARP), myoclonic epilepsy, ragged red fibers (MERRF), mitochondrial encephalopathy, lactic acidosis, stroke-like episodes (MELAS), Alzheimer's diseases (AD), Parkinson's disease (PD), Huntington's disease (HD), amyotrophic lateral sclerosis (ALS, aka Lou Gehring's diseases, Epilepsy, Autism, Fibromyalgia, chronic fatigue, cerebral palsy, Friedreich's Ataxia, Rett Syndrome, and Fragile X Syndrome, cardiomyopathy and muscular dystrophy (MD), diabetes and side effects of antibiotics treatment of bacterial infection.

The term “oxidative stress” is used in accordance with its ordinary meaning and refers to aberrant levels of reactive oxygen species.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “purified,” “substantially purified,” and “isolated” means that the composition of the invention described herein are separated from other components of either (a) a natural source, such as a plant or cell, or (b) a synthetic organic chemical reaction mixture, such as by conventional techniques. In one preferred embodiment, an “purified,” “substantially purified,” and “isolated” refers to a therapeutic muropeptide useful in the present invention being free of other, dissimilar compounds with which the compound is normally associated in its natural state, so that the compound comprises at least 0.5%, 1%, 5%, 10%, 20%, 50%, 75%, 80%, 85%, 90%, 95%, 96%, 97%, 98%, 99%, 99.5%, or 99.9% of the mass, by weight, of a given sample or composition. In one embodiment, these terms refer to the compound comprising at least 95%, 98%, 99%, or 99.9% of the mass, by weight, of a given sample or composition.

In certain embodiments “purified,” “substantially purified,” and “isolated” may refer to a single form of an therapeutic muropeptide, for example a therapeutic muropeptide 5′NAG-NAM disaccharide muropeptides with an amino acid peptide attached to NAM. In other preferred embodiment, “purified,” “substantially purified,” and “isolated” may refer to a complex mixture of therapeutic muropeptide that may each have distinct compositions. For example, a complex mixture may include therapeutic muropeptides having mono-, di-, or tri-saccharide structures with vary compositions and positions of associated peptides. In other preferred embodiment, “purified,” “substantially purified,” and “isolated” may refer to a complex mixture of therapeutic muropeptide as well as muropeptides that do not have a therapeutic effect. In this example, the complex mixture may include a bacterial extract, or fraction of a bacterial extract containing a complex mixture of different muropeptides compositions.

The term “derived from,” as used to describe a muropeptide derived from a PG that is not associated with a lipoprotein means isolating a muropeptides from a PG of bacteria. In other preferred embodiment, derived from,” as used to describe a muropeptide derived from a PG that has been synthesized in vitro. In still further embodiment, term “derived from,” as used to describe a muropeptide derived entirely in vitro, and not necessarily from a PG molecule.

As used herein, “inhibits,” “inhibition” refers to the decrease relative to the normal wild-type level, or control level. Inhibition may result in a decrease, for example ROS production, in response a therapeutic muropeptide of the invention by less than 10%, 10%, 15%, 20%, 25%, 30%, 35%, 40%, 45%, 50%, 55%, 60%, 65%, 70%, 75%, 80%, 85%, 90%, 95% or 100%.

As used herein, “increase,” “enhance” refers to the increase relative to the normal wild-type level, or control level. Increasing may result in an increase, for example ATP production, in response a therapeutic muropeptide of the invention by less than 10%, 10%, 15%, 20%, 25%, 30%, 35%, 40%, 45%, 50%, 55%, 60%, 65%, 70%, 75%, 80%, 85%, 90%, 95% or 100% or more.

In certain embodiment, a “pharmaceutically acceptable carrier” includes a “pharmaceutically acceptable salt” which refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable salts include inorganic acids such as hydrochloric acid, hydrobromic acid, sulfuric acid, and phosphoric acid; and organic acids such as oxalic acid, maleic acid, succinic acid, and citric acid. “Basic addition salts” refer to salts derived from appropriate bases, these salts including alkali metal, alkaline earth metal, and quaternary amine salts. Hence, the present invention contemplates sodium, potassium, magnesium, and calcium salts of the compounds disclosed herein, and the like. Basic addition salts can be prepared during the final isolation and purification of the compounds, often by reacting a carboxyl group with a suitable base such as the hydroxide, carbonate, or bicarbonate of a metal cation or with ammonia or an organic primary, secondary, or tertiary amine. The cations of therapeutically acceptable salts include lithium, sodium (by using, e.g., NaOH), potassium (by using, e.g., KOH), calcium (by using, e.g., Ca(OH)₂), magnesium (by using, e.g., Mg(OH)₂ and magnesium acetate), zinc, (by using, e.g., Zn(OH)₂ and zinc acetate), and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, piperazine, choline hydroxide, hydroxyethyl morpholine, hydroxyethyl pyrrolidone, imidazole, n-methyl-d-glucamine, N,N′-dibenzylethylenediamine, N,N′-diethylethanolamine, N,N′-dimethylethanolamine, triethanolamine, and tromethamine. Basic amino acids (e.g., 1-glycine and 1-arginine) and amino acids which may be zwitterionic at neutral pH (e.g., betaine (N,N,N-trimethylglycine)) are also contemplated.

The terms “administer,” “administering,” or “administration” refers to injecting, implanting, absorbing, or ingesting one or more therapeutic muropeptides, which may be part of a pharmaceutical composition.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. In a preferred embodiment, treatment may be directed towards an iron-deficiency related disorder, such as iron-deficiency anemia.

A “therapeutically effective amount” of a compound, preferably a therapeutic muropeptide, of the present invention or a pharmaceutical composition thereof is an amount sufficient to provide a therapeutic benefit in the treatment of a disease or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, and/or enhances the therapeutic efficacy of another therapeutic agent. A “therapeutically effective amount” may also mean “prophylactically effective amount” of a compound of the present invention is an amount sufficient to prevent a disease or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “pharmaceutical composition” or “pharmaceutical composition of the invention” refers to a composition of the invention, and preferably a therapeutic muropeptide composition of the invention, or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients. In other embodiments, the pharmaceutical composition further comprises at least one additional anticancer therapeutic agent, such as through a co-treatment. As used herein, a “pharmaceutically acceptable carrier” refers to a carrier or diluent that does not cause significant irritation to an organism and does not abrogate the biological activity and properties of the administered composition of the invention. The pharmaceutical acceptable carrier may comprise any conventional pharmaceutical carrier or excipient. The choice of carrier and/or excipient will to a large extent depend on factors such as the particular mode of administration, the effect of the carrier or excipient on solubility and stability, and the nature of the dosage form.

Suitable pharmaceutical carriers include inert diluents or fillers, water, and various organic solvents (such as hydrates and solvates). The pharmaceutical compositions may, if desired, contain additional ingredients such as flavorings, binders, excipients, and the like. Thus, for oral administration, tablets containing various excipients, such as citric acid may be employed together with various disintegrants such as starch, alginic acid and certain complex silicates and with binding agents such as sucrose, gelatin, and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension, or emulsion, for topical administration as an ointment or cream or for rectal administration as a suppository. The pharmaceutical composition may be in unit dosage forms suitable for single administration of precise dosages.

Exemplary parenteral administration forms include solutions or suspensions of active compounds in sterile aqueous solutions, for example, aqueous propylene glycol or dextrose solutions. Such dosage forms may be suitably buffered, if desired.

A pharmaceutical composition of the invention may be administered as single agents, for example a pharmaceutical composition of a composition of the invention, or a pharmaceutical composition of a bivalent memetic peptide of the invention, or may be administered in combination with other anti-cancer therapeutic agents, in particular standard of care agents appropriate for the particular cancer. In some embodiments, the methods provided result in one or more of the following effects: (1) inhibiting cancer cell proliferation; (2) inhibiting cancer cell invasiveness; (3) inducing apoptosis of cancer cells; (4) inhibiting cancer cell metastasis; or (5) inhibiting angiogenesis. Pharmaceutical compositions suitable for the delivery of compounds of the invention, such as a memetic peptide as described herein, and methods for their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

Relative amounts of the active ingredient, the pharmaceutically acceptable carriers or excipients, and/or any additional ingredients in a pharmaceutical composition of the invention will vary, depending upon the identity, size, and/or condition of the subject treated and further depending upon the route by which the composition is to be administered. The composition may comprise between 0.1% and 100% (w/w) active ingredient.

Pharmaceutically acceptable carriers or excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly (vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly (vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs which, preferably contain a unit dosage of one or more therapeutic muropeptides. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically, contemplated routes of administration of the compounds and compositions disclosed herein are inhalation and intranasal administration, subcutaneous administration, mucosal administration, and interdermal administration. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of an “active ingredient” required to achieve an effective amount will vary from subject to subject, depending, for example, on species, age, and general condition of a subject, severity of the side effects or disorder, identity of the particular compound, mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

Administration of a composition of the invention, and preferably a therapeutic muropeptide of the invention, may be effected by any method that enables delivery of the compositions to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound, for example a composition of the invention, and preferably a therapeutic muropeptide composition of the invention, calculated to produce the desired therapeutic effect in association with the required pharmaceutical carrier. The specification for the dosage unit forms of the invention are dictated by and directly dependent on (a) the unique characteristics of the composition and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The amount of a composition of the invention, and preferably a therapeutic muropeptide composition of the invention, administered will be dependent on the subject being treated, the severity of the disorder or condition, the rate of administration, the disposition of the compound and the discretion of the prescribing physician. However, an effective dosage is typically in the range of about 0.001 to about 100 mg per kg body weight per day, preferably about 0.01 to about 35 mg/kg/day, in single or divided doses. For a 70 kg human, this would amount to about 0.07 to about 7000 mg/day, preferably about 0.7 to about 2500 mg/day. In some instances, dosage levels below the lower limit of the aforesaid range may be more than adequate, while in other cases still larger doses may be used without causing any harmful side effect, with such larger doses typically divided into several smaller doses for administration throughout the day. In one preferred embodiment, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.1 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

In one preferred embodiment, a therapeutically effective amount or dosage of a composition of the invention, and preferably a therapeutic muropeptide composition of the invention, may be a dosage sufficient to inhibit ROS formation in a subject, decrease oxidative stress in a subject, increase ATP production in a subject or assay, stabilize ATP synthase complex in a subject or assay, or increase ATP synthase activity in a subject or assay.

Also encompassed by the invention are kits (e.g., pharmaceutical packs). The kits provided may comprise a therapeutic muropeptide composition (e.g., pharmaceutical or diagnostic composition) and a container (e.g., a vial, ampule, bottle, syringe, and/or dispenser package, or other suitable container). The kits provided may comprise antibodies that selectively bind a therapeutic muropeptide (e.g., pharmaceutical or diagnostic composition) and a container (e.g., a vial, ampule, bottle, syringe, and/or dispenser package, or other suitable container). In some embodiments, provided kits may optionally further include a second container comprising an excipient (e.g., pharmaceutically acceptable carrier) for dilution or suspension of an inventive pharmaceutical composition or compound. In some embodiments, the therapeutic muropeptide composition provided in the first container and the second container are combined to form one unit dosage form. In another embodiment, the present invention provides kits including a first container comprising antibodies produced using the therapeutic muropeptide, i.e., antibodies that selectively bind a therapeutic muropeptide.

The term “subject” refers to any animal. In certain embodiments, the subject is a mammal. In certain embodiments, the subject is a human (e.g., a man, a woman, or a child). The human may be of either sex, or may be at any stage of development. In certain embodiments, the subject has been diagnosed with the mitochondrial condition or disease to be treated. In other embodiments, the subject is at risk of developing the mitochondrial condition or disease. In certain embodiments, the subject is an experimental animal (e.g., mouse, rat, rabbit, dog, pig, or primate). The experimental animal may be genetically engineered. In certain embodiments, the subject is a domesticated animal (e.g., dog, cat, bird, horse, cow, goat, sheep, or chicken).

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the term “contacting” means bringing together of two elements in an in vitro system or an in vivo system. For example, “contacting” a compound disclosed herein with an individual or patient or cell includes the administration of the compound to an individual or patient, such as a human, as well as, for example, introducing a compound into a sample containing a cellular or purified preparation containing the compounds or pharmaceutical compositions disclosed herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

The invention now being generally described will be more readily understood by reference to the following examples, which are included merely for the purposes of illustration of certain embodiments of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Novel Therapeutic Application of Muropeptides as Class of Compounds to Treat a Disease or Condition

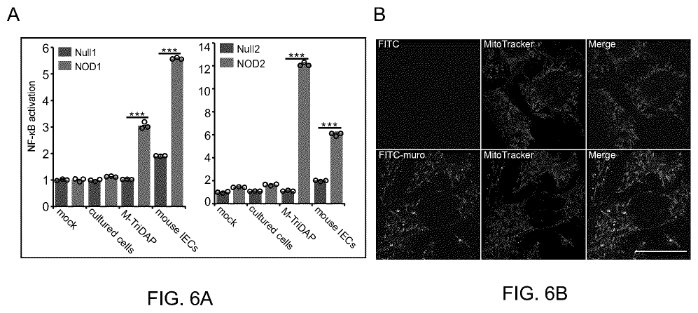

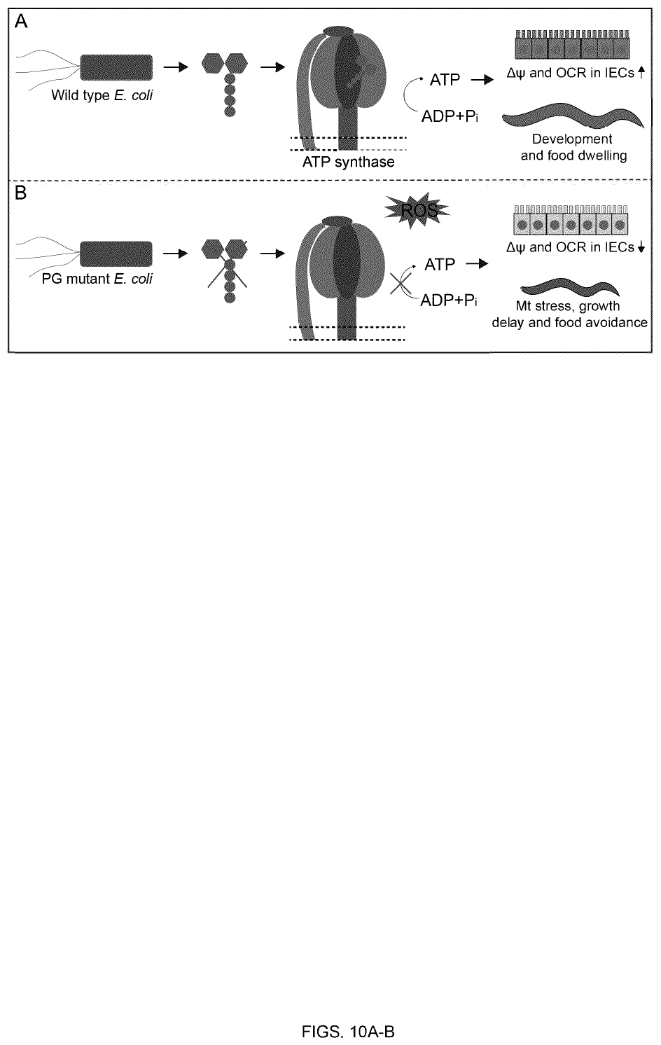

The present inventive technology is directed to a previously unknown beneficial and/or nutritional role of bacterial muropeptides in regulating host physiology and insights of the underlying mechanism (FIG. 10). We provide evidence that the muropeptides from the exemplary bacteria E. Coli act to promote post-embryonic development and food dwelling behavior and do so at least in part by entering the Mt of intestinal cells and binding to ATP synthase. Our data also suggest that the changes in oxidative stress and UPR^{mt} are largely responsible for the PG muropeptide-related phenotype changes in postembryonic development and food behaviors in C. elegans. Given the abundant presence of commensal gut bacteria across the animal kingdom including in humans and exceptional structural conservation of ATP synthase, the beneficial usage of the bacterial muropeptides, having common structural characteristics, by the host can be conserved in mammals. This notion is supported by the observation that that PG muropeptides also accumulate in the Mt of mouse IEC cells (FIG. 6) and bind to ATP Synthase in human caco-2 cells (FIGS. 9A-C). Importantly, supplementation of muropeptides promotes ATP synthase activity and ATP-linked Mt oxidative respiration of human caco-2 cells (FIGS. 9D-I). As a result, the present inventive technology includes an unexpected beneficial role of an individual bacterial metabolite and the fascinating underlying mechanism, which also provides valuable insights into the symbiotic relationship between the host animals and commensal microbes and adds to the evidence that the Mt is an important hub for host-microbiome interaction.

Previous studies have identified numerous protein and chemical inhibitors of ATP synthase and shown the profound impact of the inhibition on Mt ROS production, cellular metabolism, aging and cancer cell progression (Alavian et al., 2011; Chin et al., 2014; Esparza-Molto and Cuezva, 2018; Goldberg et al., 2018; Hong and Pedersen, 2008). However, since no effective ATP synthase agonists have been previously described, the present inventive describes the first effective ATP synthase agonist and that this agonist is normally present in animal intestine cells to execute such a role. This interaction between PG muropeptides and ATP synthase may be a powerful tool to address a variety questions regarding Mt homeostasis, energy balance, as well as for the treatment of versions diseases and conditions. Additional embodiment may have relevance as a molecular diagnostic tool that may require an effective ATP synthase agonist.

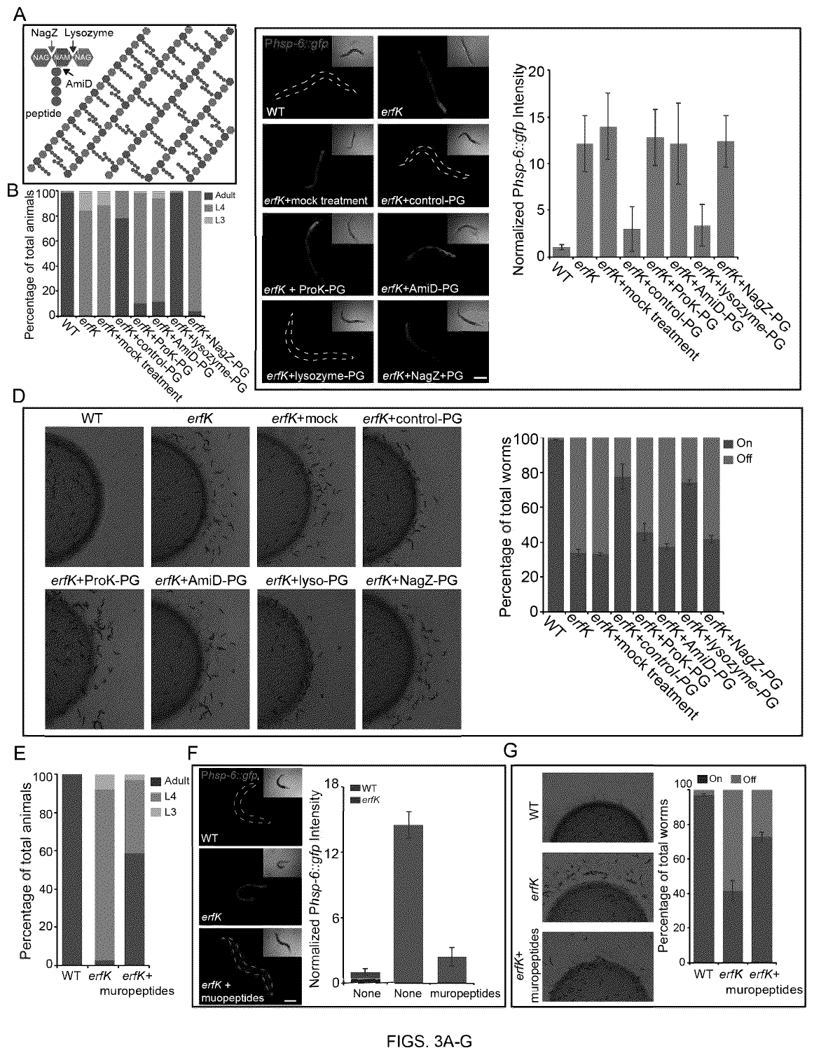

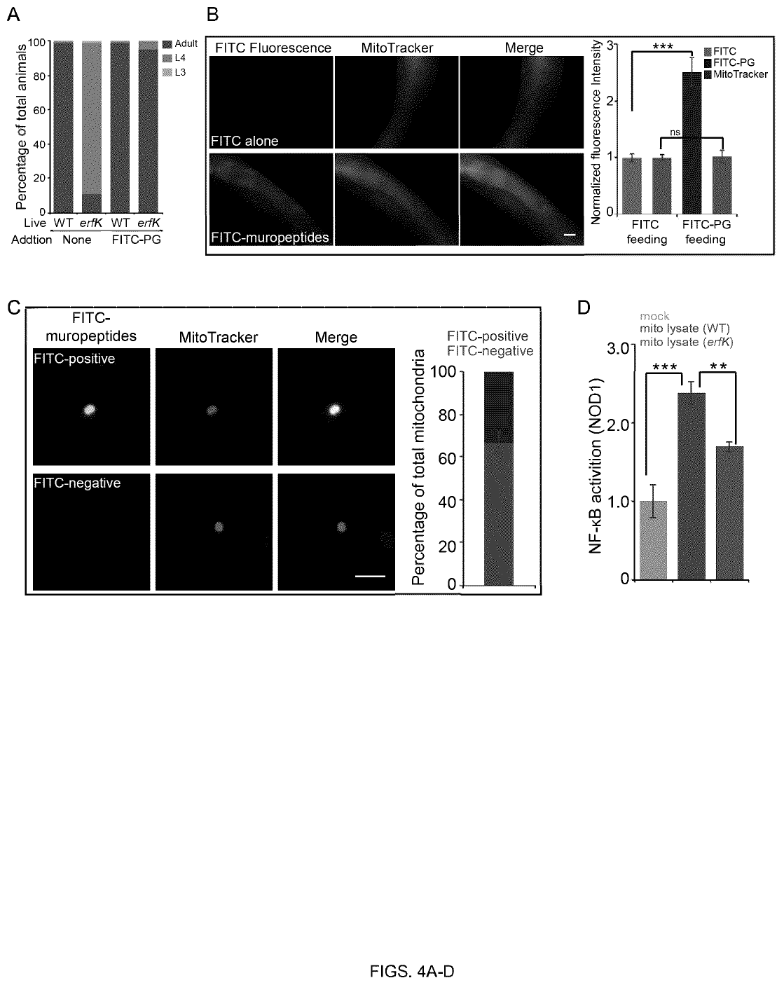

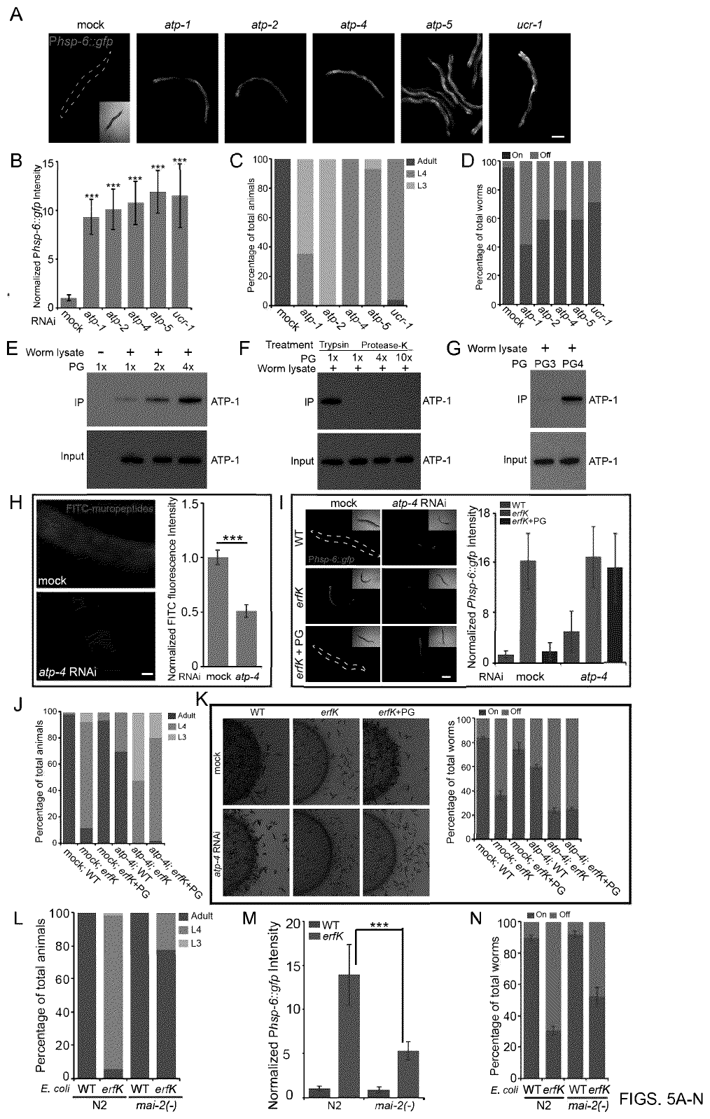

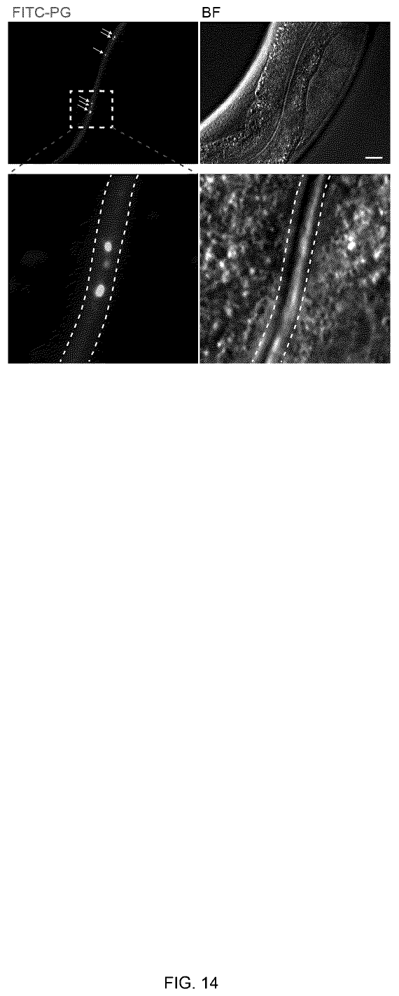

Notably, the isolated PG are large molecules that are not likely absorbed directly by the host cells. Indeed, in the FITC-PG feeding assay, we only observed PG puncta (large molecules) in intestinal lumen but not inside of Mt or cells of the intestine (FIGS. 4B, 4C and 14). This observation is consistent with the model that the functional molecules are muropeptides that are the breakdown products from PG. This conclusion is based on our functional assays showing that muropeptides from lysozyme-treated PG are sufficient to suppress Mt stress, developmental delay and food avoidance caused by feeding PG defective bacteria to C. elegans (FIGS. 3E-3G), as well as to promote ATP synthase activity, Mt oxidative respiration in mammalian intestinal epithelial cells (FIGS. 6D-61). In the intestine, PG may be broken down into muropeptides by the combination of two processes. First, large PG molecules may be hydrolyzed by enzymes produced by the host animals. Animal genomes contain a number of highly-conserved lysozyme genes (e.g., lys-1-10 and ilys-1-6 in C. elegans), and some are expressed constitutively in the intestine to digest PG. Second, during bacterial proliferation, partial degraded PG molecules are released into the environment. As indicated by the defects of the 4 PG mutant E. coli described herein, activities of certain PG metabolic enzymes are required to generate PG molecules that can be further processed to produce functional muropeptides.

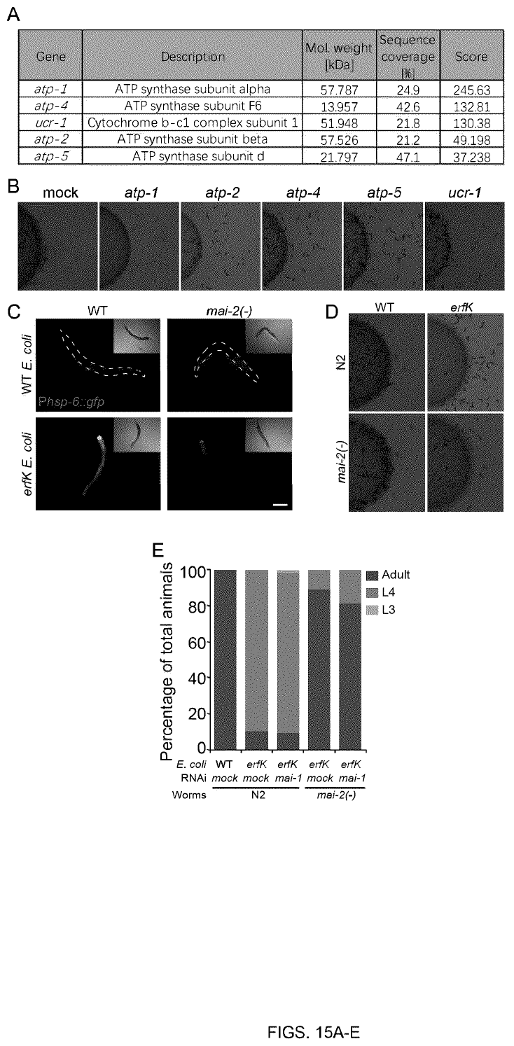

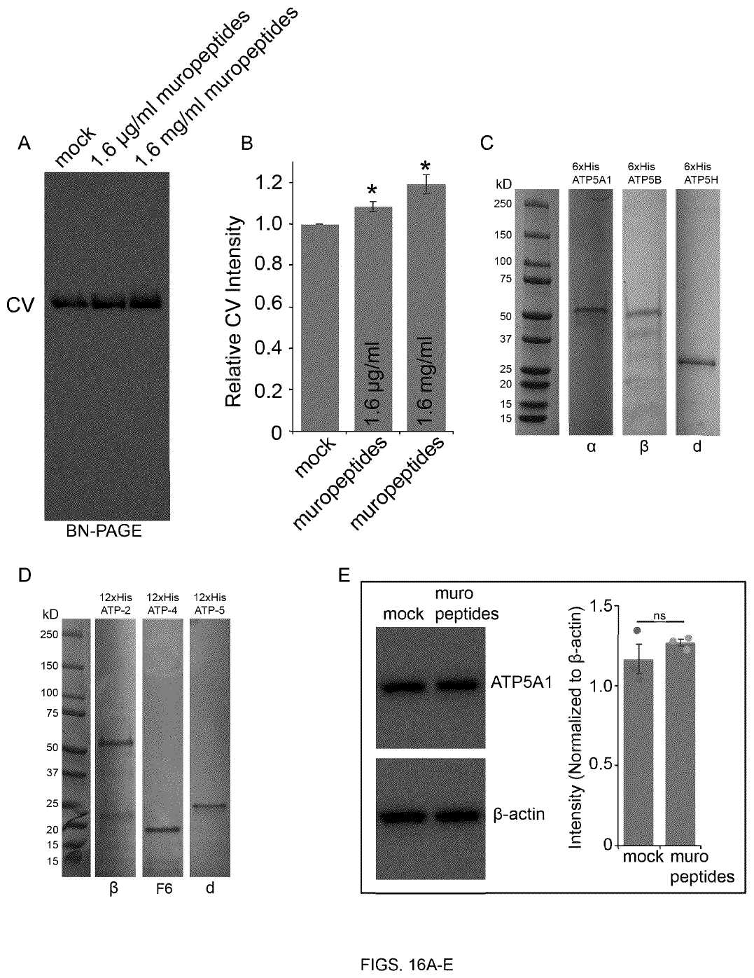

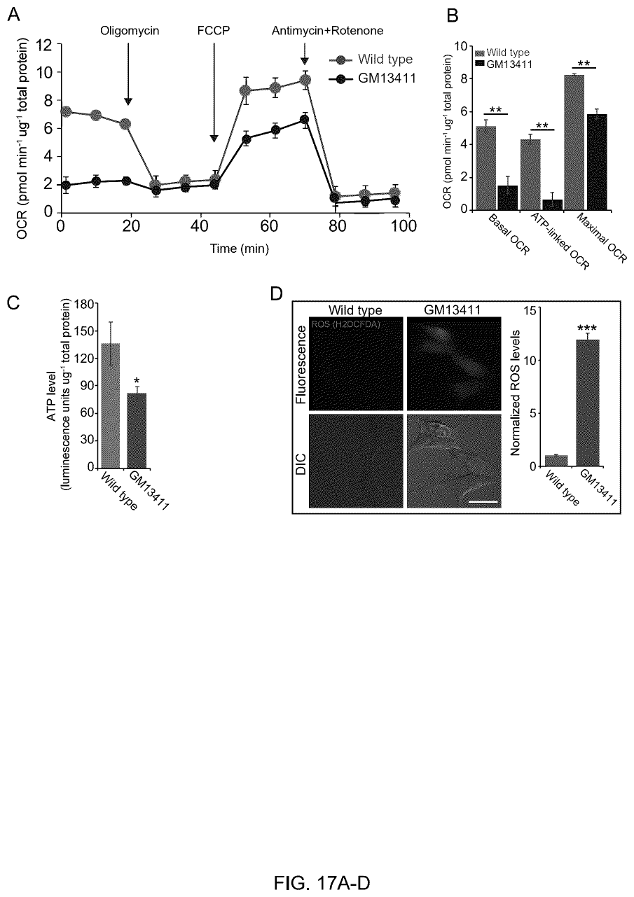

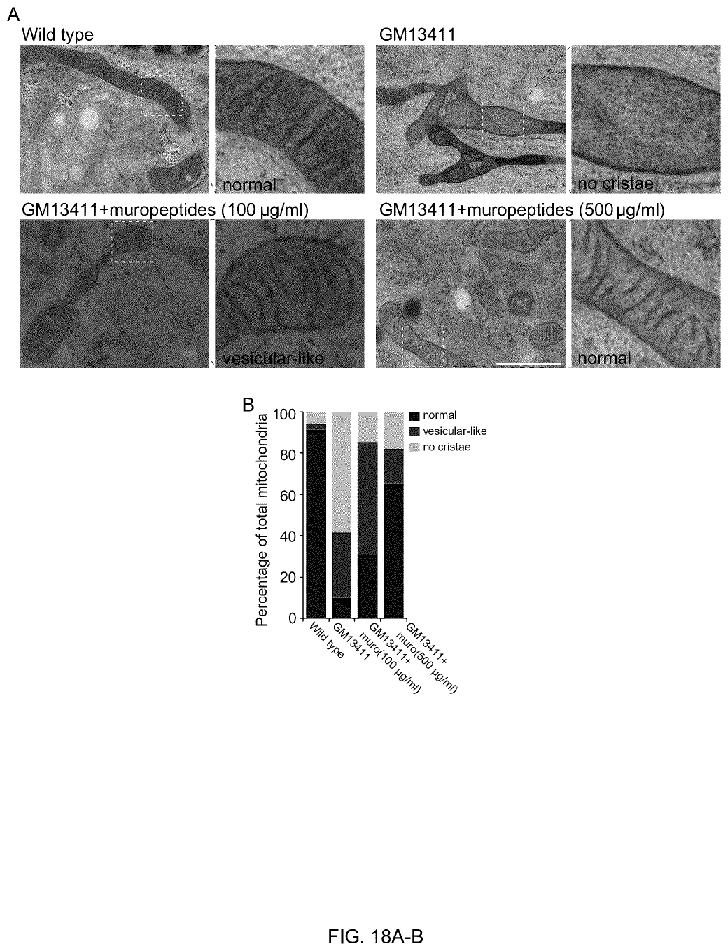

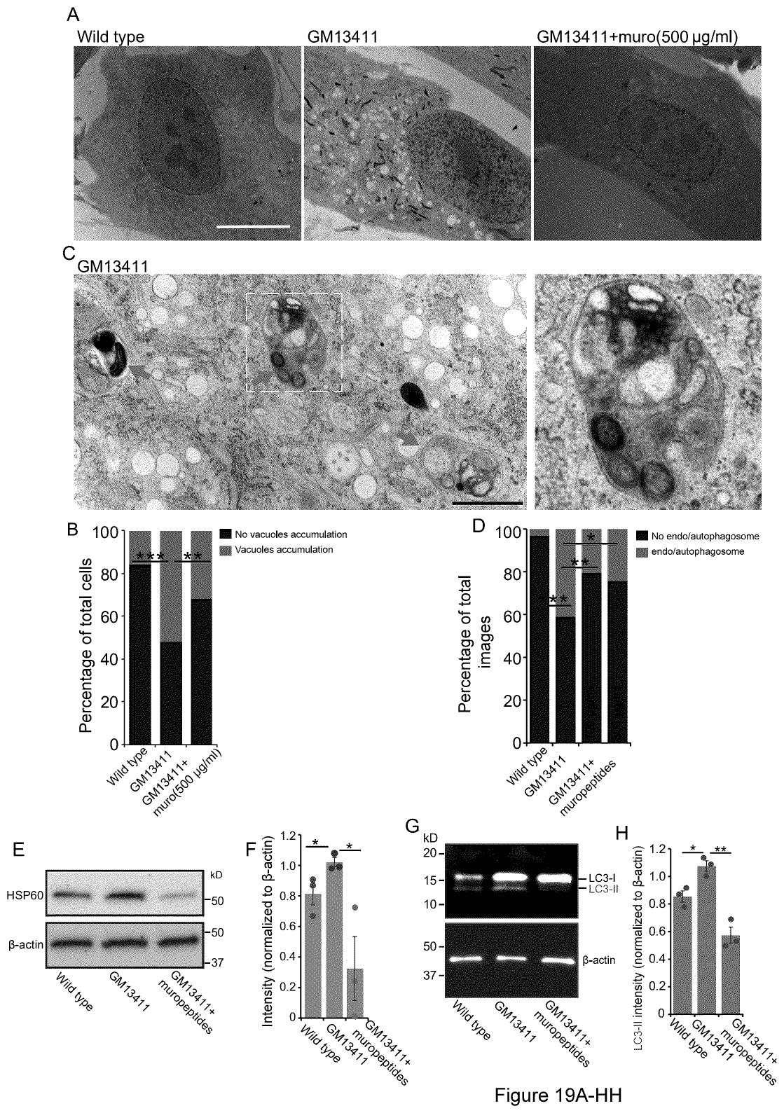

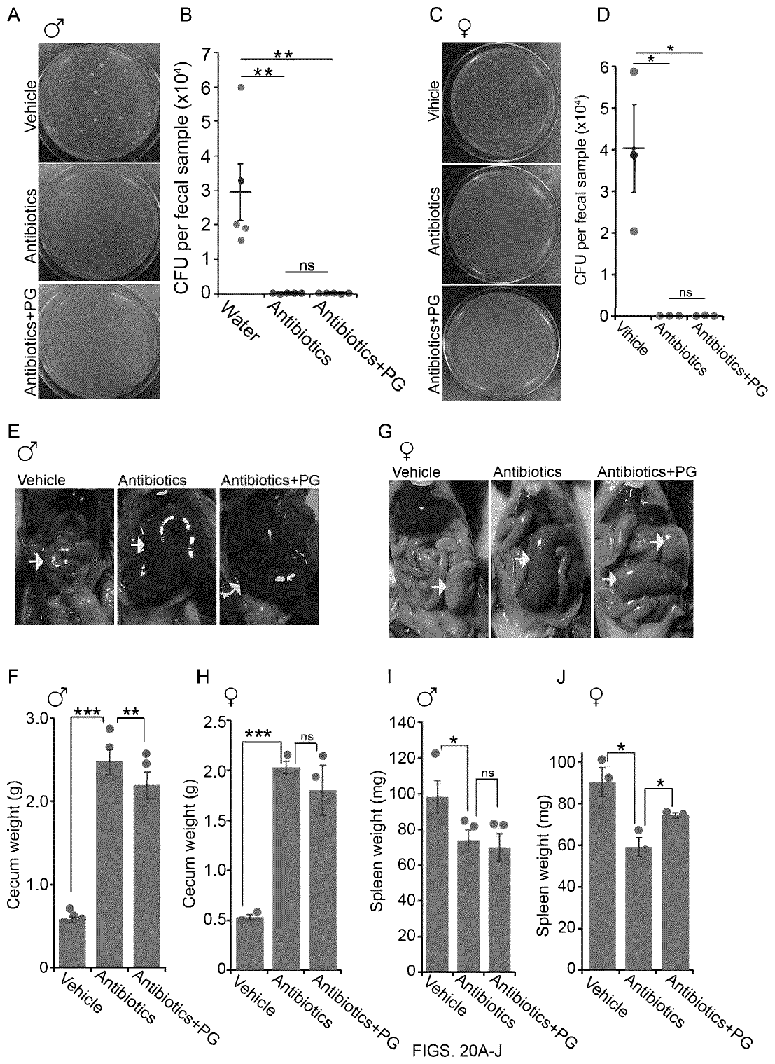

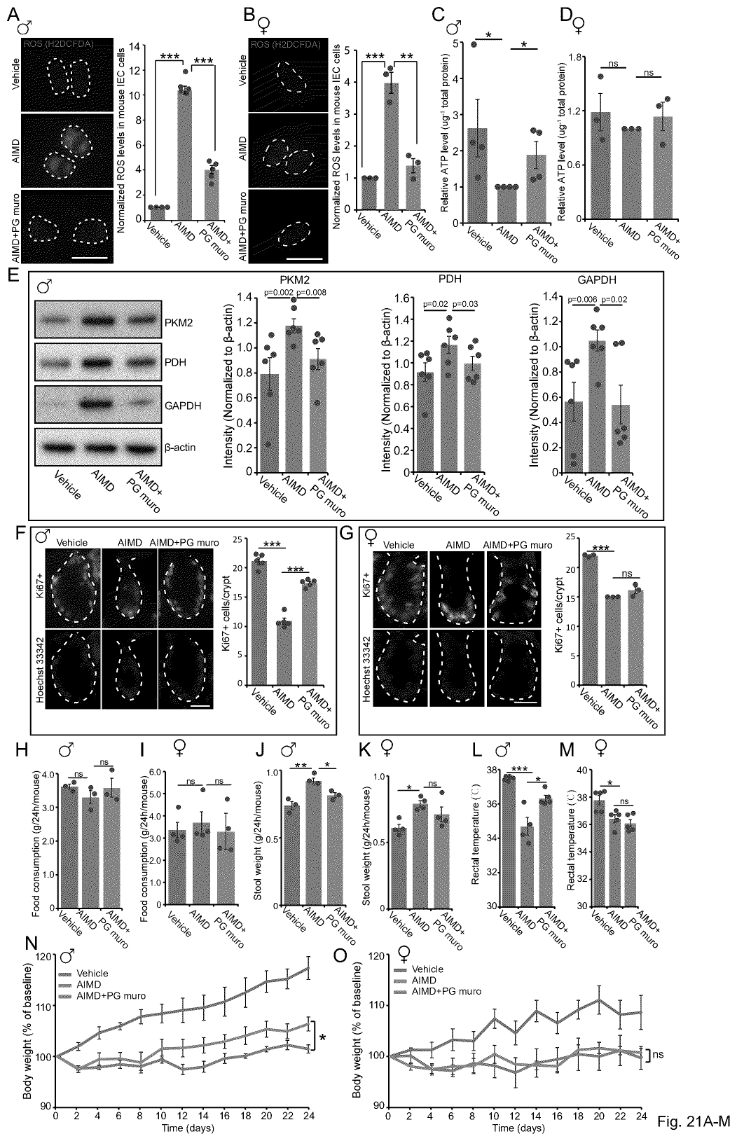

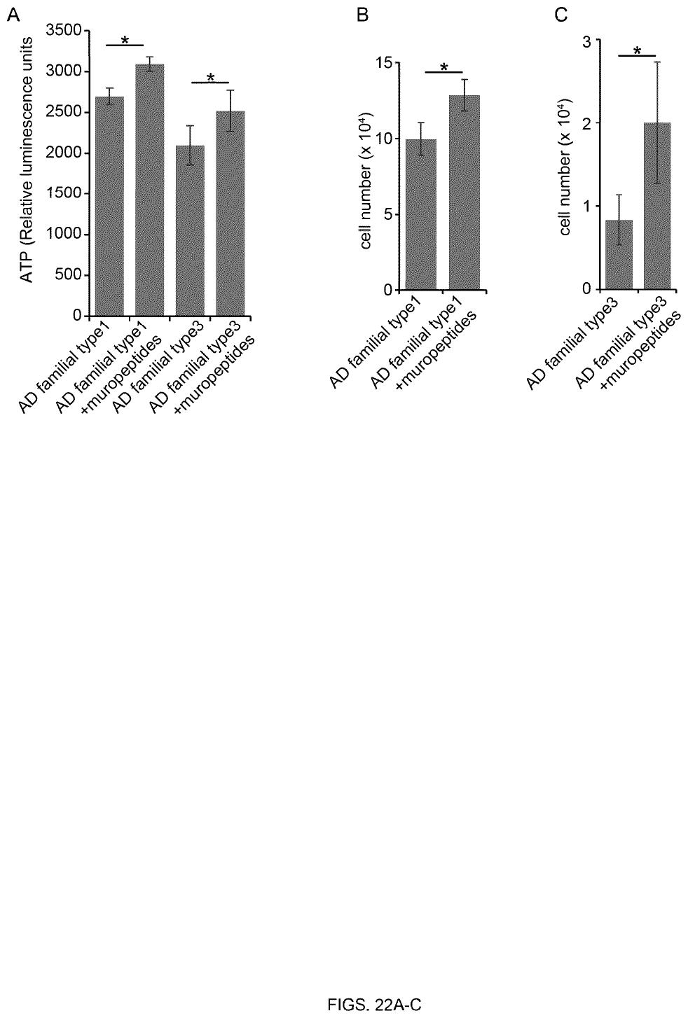

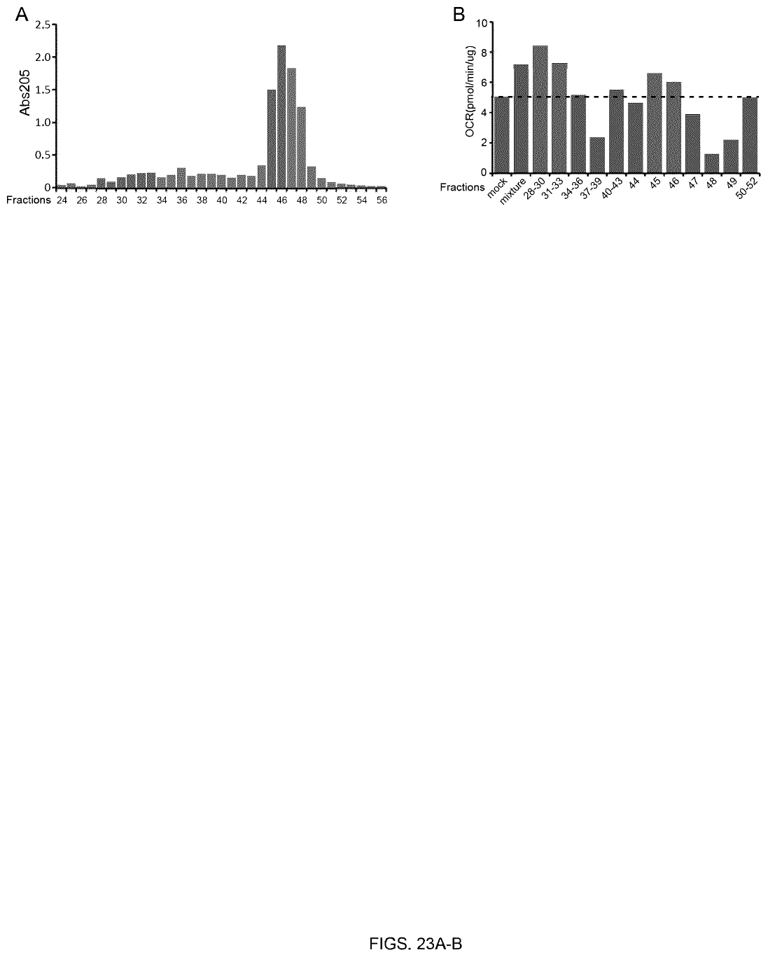

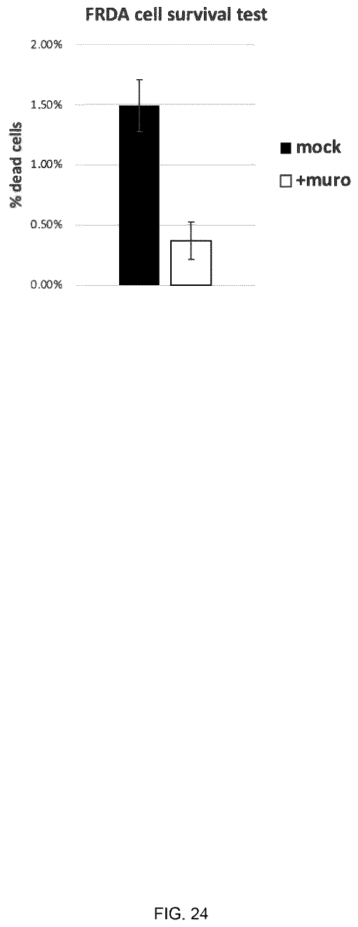

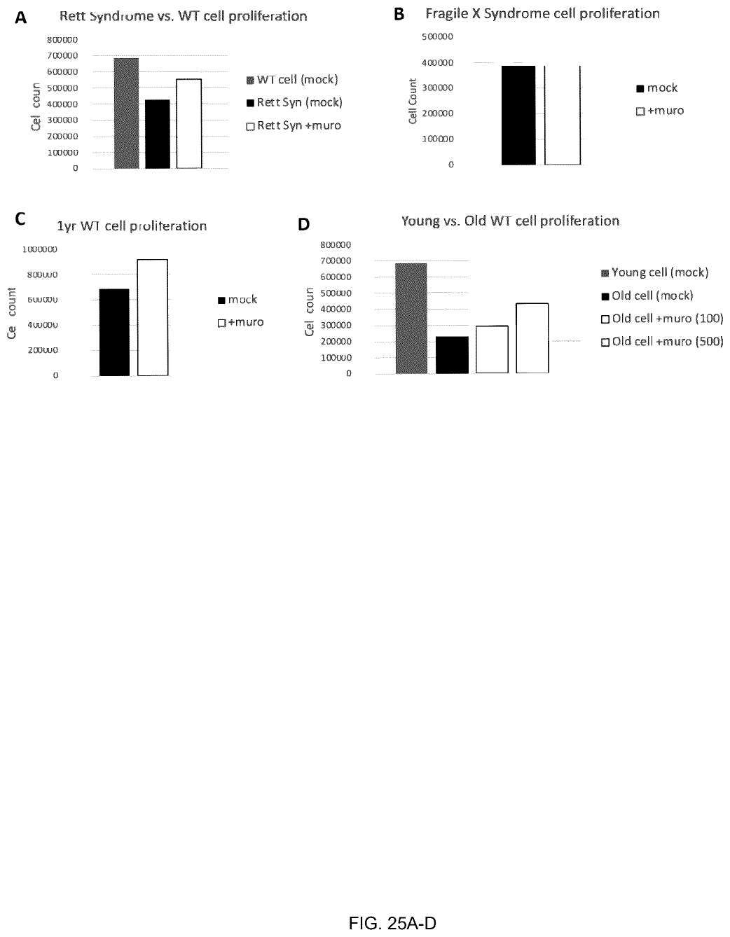

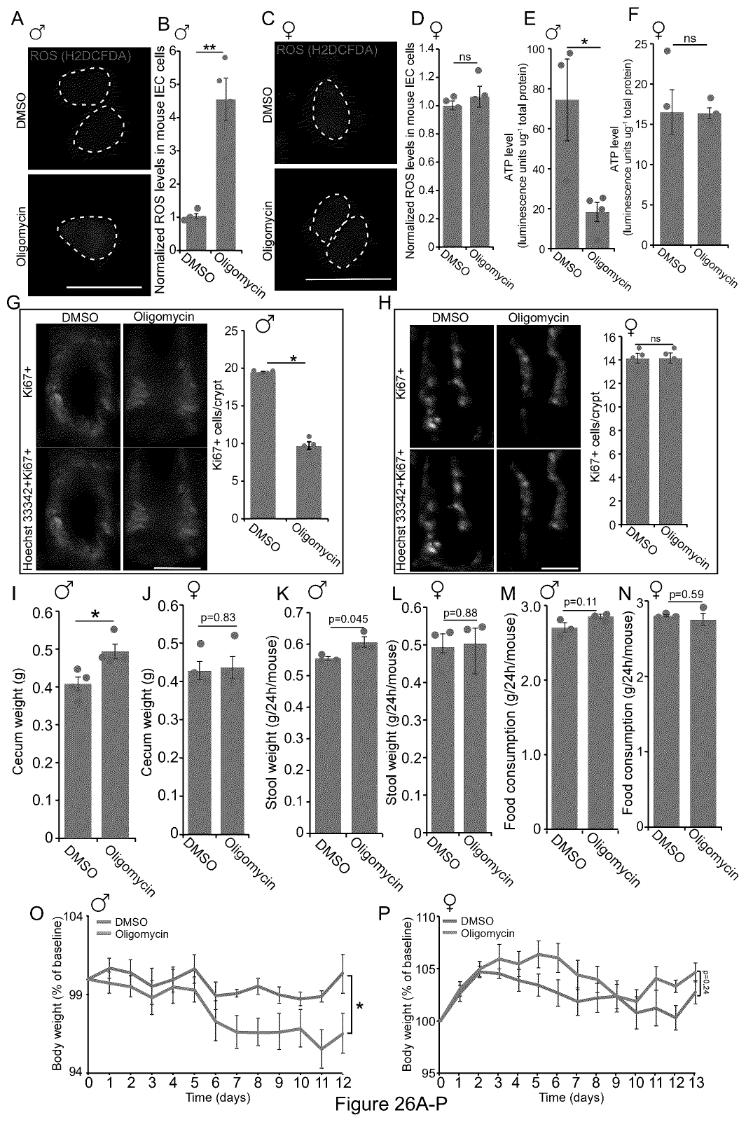

Besides ATP synthase subunits, the combination of PG pull-down assay and RNAi analysis identified another Mt protein UCR-1, which is a complex III subunit of electron transport chain (FIGS. 5A-5D and 15A and 15B). This interaction may suggest that the muropeptides can positively influence Mt oxidative respiration through interaction with multiple components of the electron transport chain, whose activity is dynamically regulated to meet cellular metabolic status.

### Example 2: Bacterial PG Metabolites Support Development and Food Behavior in C. elegans

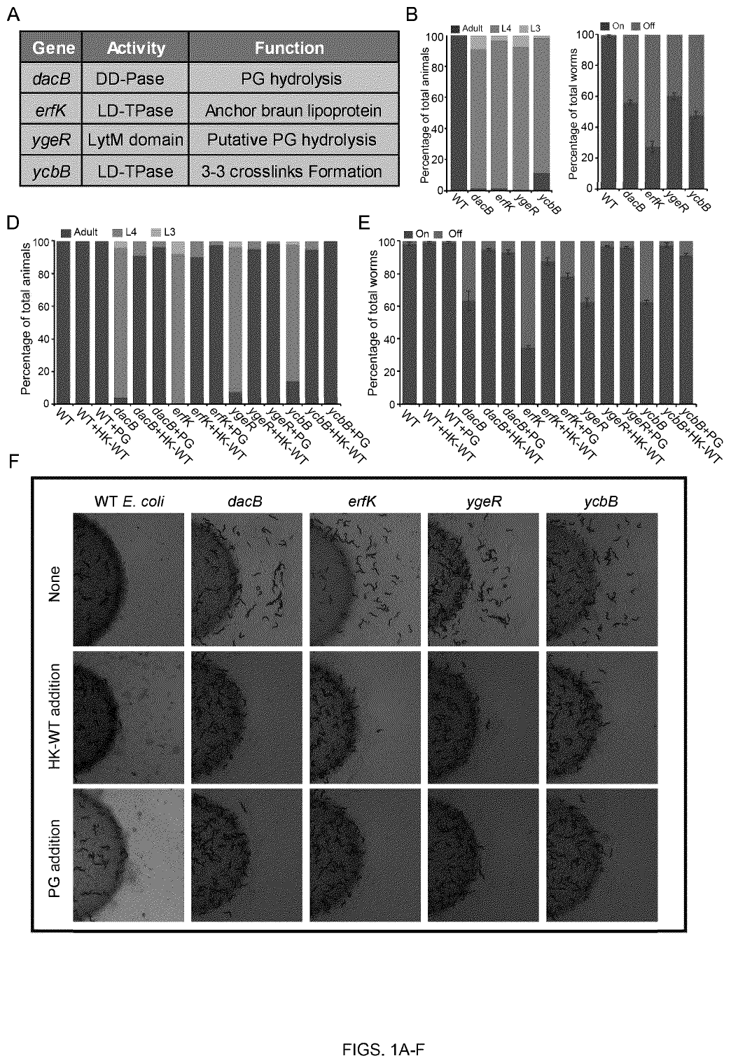

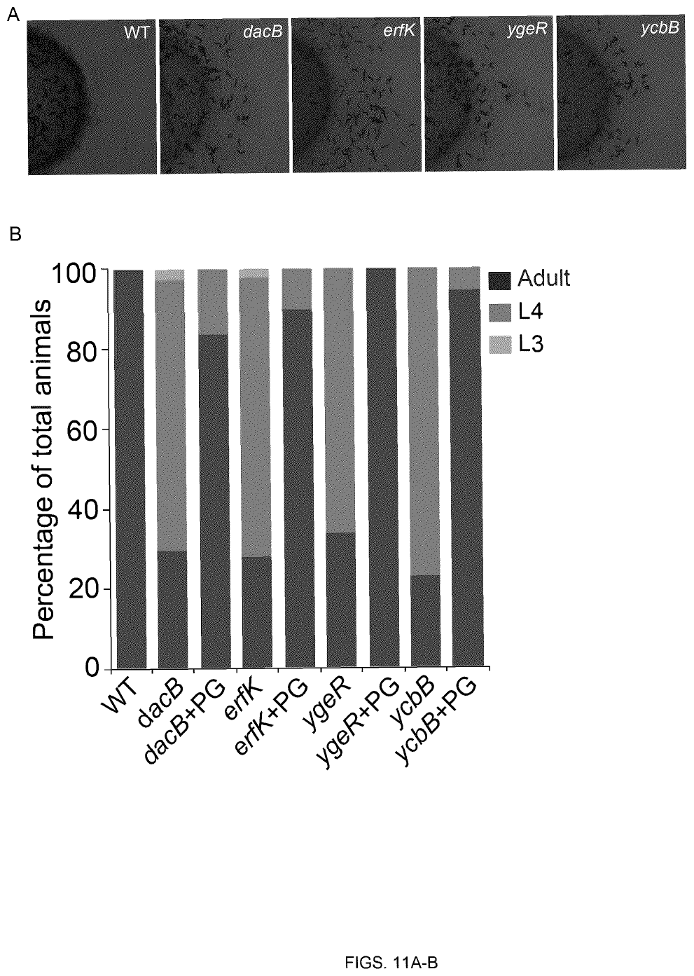

To search for beneficial impacts of individual bacterial metabolites on animals, the present inventors performed genetic screens of the Keio collection of E. coli for developmental and behavior defects in C. elegans using multiple assay conditions. After identifying a PG-related mutant during a pilot screen using an established assay, we assembled and screened a sub-library that contains 57 PG metabolism mutants (Table 1). We found that worms fed 4 mutant bacterial strains (called PG mutants thereafter) displayed significant developmental delay (FIGS. 1A and 1B). Two of these mutants (erfK and ycbB) were also among candidates from an independent screen of the Keio collection for developmental delay. Previous studies have linked developmental delay to food avoidance (or lack of food dwelling) behaviors. We thus carried out a simple assay and found that feeding each of the PG mutants to C. elegans also triggered food avoidance behavior (FIGS. 1C and 11A). These data suggest that proper PG metabolism in E. coli have significant effects on host physiology.

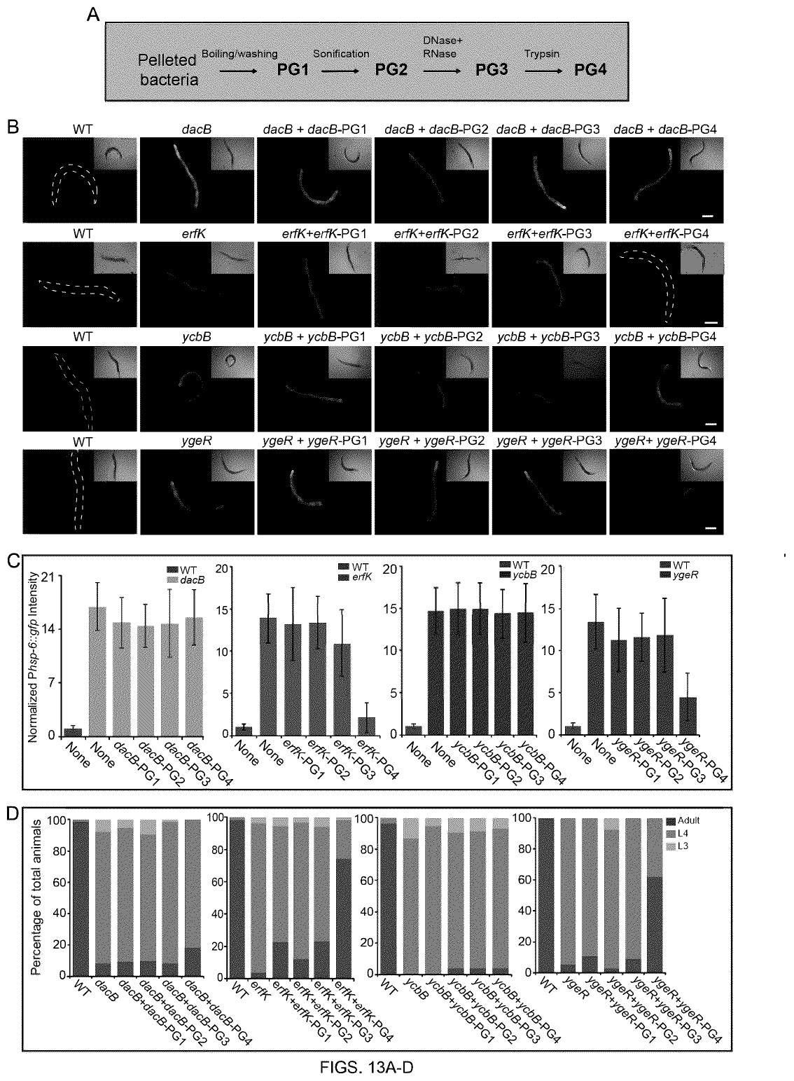

Both the developmental delay and food avoidance behavior can be caused by either the presence of toxic molecules or the absence of beneficial metabolites. One effective way to distinguish between these possibilities is to supplement the PG mutant with PG molecules isolated from wild-type E. coli and examine a potential rescue effect. We first supplemented heat-killed wild type (HK-WT) E. coli, which contains PG molecules, to worms fed live PG mutant E. coli, and found that the developmental delay and food avoidance behavior were almost completely overcome (FIGS. 1D-1F). We then isolated PG from wild type bacteria following a published protocol (FIG. 13A), and found that these PG molecules are highly effective in overcoming the developmental delay and food avoidance phenotypes (FIGS. 1D-1F).

To determine if the causes of the negative effects from feeding all 4 PG mutant E. coli are related, we fed animal with the mixture of two PG mutants of equal amounts. We observed that these double mutant E. coli mixtures induced growth delay at similar levels as single mutant E. coli feeding (FIG. 11B). Together, the above results suggest that lack of common PG-related molecules in these PG mutants cause the defects in host animals, which is supported by further assays described below.

### Example 3: Deficiency in Beneficial PG Metabolites Induces Mt Stress in C. elegans

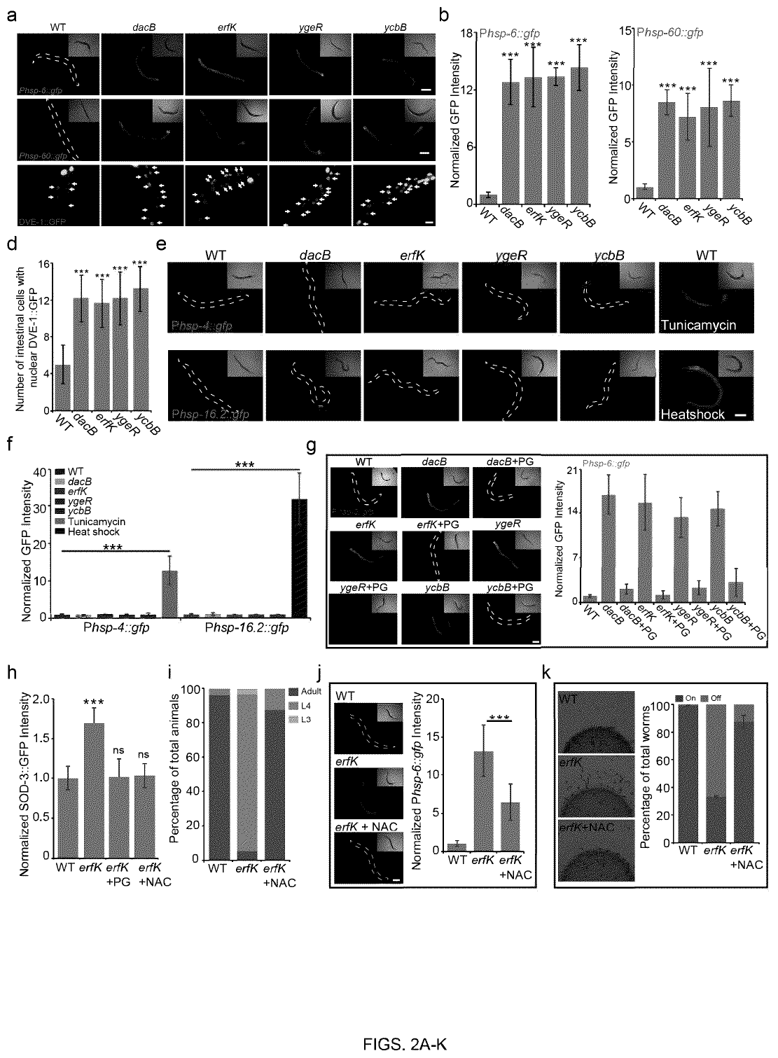

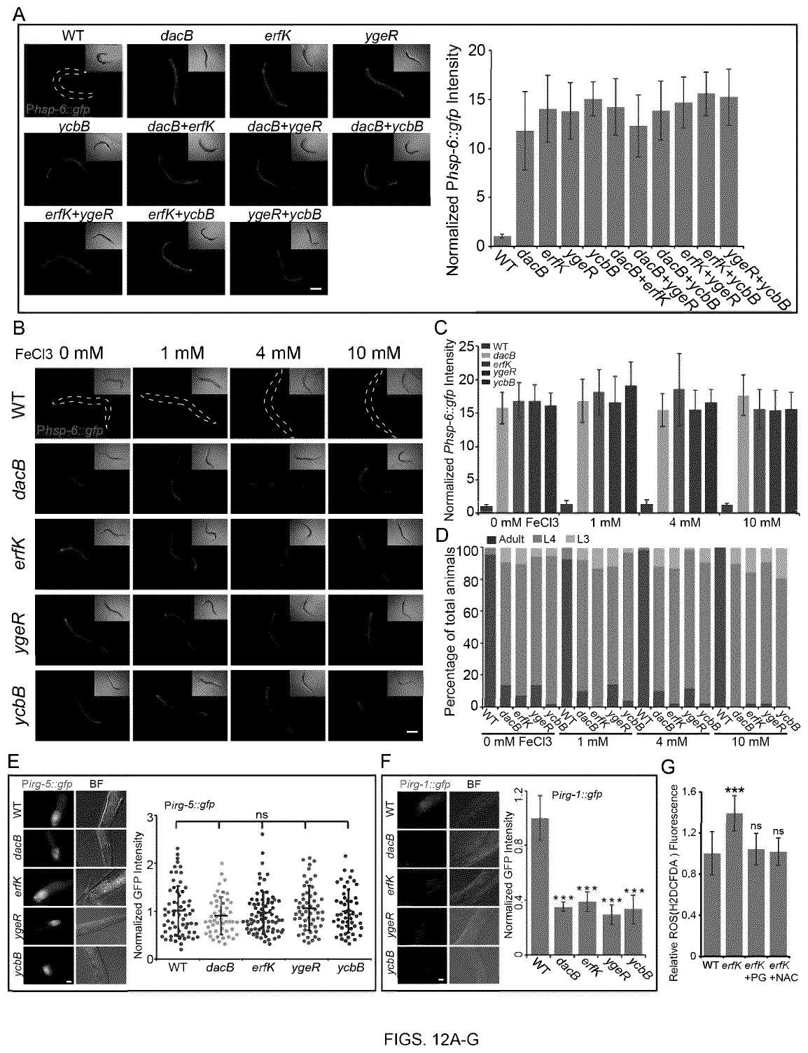

Cellular stresses and unfolded protein responses (UPR) are induced by environmental factors including bacterial metabolites. These stresses are known to play roles in animal development and food behaviors (Liu et al., 2014; Melo and Ruvkun, 2012). We examined the levels of several commonly used stress reporters in worms fed with these PG mutant E. coli, and found that Mt stress (Phsp-6::gfp and Phsp-60::gfp), but not ER stress (Phsp-4::gfp) nor cytoplasmic stress (Phsp-16.2::gfp), were strongly induced (FIGS. 2A-2C, 2E and 2F). Further support of Mt stress induction came from analysis of DVE-1, a critical co-factor in ATFS-1 mediated UPR^{mt} that is translocated into the nucleus upon Mt stress. We found that PG mutants feeding also promoted nuclear accumulation of DVE-1::GFP in the intestinal cells (FIGS. 2A and 2D). Importantly, we found that PG molecules isolated from wild-type E. coli also effectively suppressed Mt stress in C. elegans fed with each of the 4 PG mutants (FIG. 2G). In addition, feeding worms with a mixture of two PG mutants induced UPR^{mt} at similar levels as feeding with single mutants (FIG. 12A). These data also support that these PG mutants lack common, beneficial PG-related molecules. We also performed additional tests to show that the observed developmental defects of worms fed with these PG mutants are not likely due to iron deficiency or innate immune response abnormality (Girardin et al., 2003; Gottar et al., 2002; Irazoki et al., 2019; Royet et al., 2011; Zhang et al., 2019) (FIGS. 12B-12F).

Moreover, recent studies showed that iron supplementation can rescue developmental delay and UPR^{mt} in C. elegans caused by feeding certain bacterial mutants (Zhang et al., 2019). Our tests indicated that iron supplementation could not suppress the Mt stress or growth delay for any of the four PG mutants (B-D), suggesting that the observed defects in C. elegans are not due to iron deficiency. Previous studies also indicated that certain PG and PG-derived muropeptide contribute to triggering host innate immune responses. Using two established reporters, we found no significant change of p38-dependent reporter activity (Pirg-5::gfp) and reduction of the expression of a p38-independent reporter (Pirg-1::gfp) (rather than increased expression that would indicate an immune response) in worms fed with PG mutants (E and F). These data are consistent with the notion that feeding worms with the PG mutant E. coli specifically induces Mt stress and UPR^{mt} and are inconsistent with a critical involvement of the innate immune response pathways in the impact of the PG mutant bacteria on the observed animal functions.

### Example 4: The Beneficial Role of PG is Largely Mediated by Suppression of Mt Oxidative Stress

Mt are the major endogenous source of reactive oxygen species (ROS), especially under stress conditions (Dingley et al., 2010; Lee et al., 2010). The sod-3 gene, encoding a Mt manganese superoxide dismutase, is a known oxidative stress response gene. Using a SOD-3::GFP reporter, we observed a significantly increased level of SOD-3 expression in C. elegans fed erfK mutant E. coli compared to control (FIG. 2H). Importantly, the increase in SOD-3 level was eliminated by adding isolated PG or antioxidant NAC (N-acetyl-L-cysteine) (FIG. 2H), which was also confirmed by a ROS staining assay (FIG. 12G), indicating a role of the beneficial PG molecules in suppressing ROS production in Mt. Moreover, we found supplementation of antioxidant NAC significantly suppressed the developmental delay, food avoidance and Mt stress induced by feeding worms with PG mutant E. coli (FIGS. 21-2K), supporting the idea that suppressing Mt oxidative stress is critically involved in the protective role of the PG molecules.

### Example 5: Beneficial Muropeptides Contain Disaccharides with Short Peptides

To learn more about the structural properties of the beneficial PG molecules, we employed several enzymes, including those that cleave PG at specific sites (FIG. 3A). We first treated PG with Protease-K, which is a broad-spectrum protease, and found that the rescue effect of PG was abolished (FIGS. 3B-3D, ProK-PG). In contrast, treatment with trypsin, a serine protease used in the final step of the PG isolation protocol (FIG. 13A), preserved the rescue effect (FIGS. 3B-3D, control-PG). We then treated the PG with Amidase (AmiD) that cleaves the short amino acid peptides from PG (FIG. 3A) and found that the rescue effect of PG was eliminated (FIGS. 3B-3D, AmiD-PG). These results suggest that the proposed beneficial molecules do not contain lipoproteins known to attach to PG (trypsin sensitive), whereas the attachment of the short amino acid peptides to PG molecules (Protease-K sensitive, Amidase-sensitive, but trypsin insensitive) are needed for the beneficial effect.

We then further examined the structure of the beneficial PG molecule by treating the active PG mix with lysozyme that cuts the bond between the 5′ N-acetylmuramic acid (NAM) and 3′ N-acetylglucosamine (NAG) or N-acetyl-glucosaminisase (NagZ) that cuts the bond between the 5′NAG and 3′NAM (FIG. 3A). Either treatment is expected to generate disaccharide-peptide molecules (disaccharide containing muropeptides). While the lysozyme treatment did not significantly affect the role of PG mix in suppressing Mt stress and growth delay in C. elegans fed erfK mutant E. coli, the NagZ treatment effectively eliminated such a suppression role (FIGS. 3B-3D, lysozyme-PG and NagZ-PG). To further confirm the efficiency of the muropeptides, we collected the supernatant from lysozyme treated PG and found that the muropeptides in the supernatant are sufficient to suppress the host phenotypes (FIGS. 3E-3G). These results suggest that the effective PG molecules are likely 5′NAG-NAM disaccharide muropeptides with an amino acid peptide attached to NAM. Further analyses using mixtures of products of specific PG purification steps from the mutants support that certain PG hydrolysis activities including removal of lipoproteins attached to PG may be involved in releasing the beneficial PG muropeptides (FIGS. 13A-D). Additional analysis of PG muropeptides fractionated from Bio-6P gel suggests that the functional PG fragments may include dimers and oligomers of the disaccharide muropeptides (FIG. 23).

### Example 6: Muropeptides Accumulate in Intestinal Mt

Since feeding PG mutant diet specifically induced Mt stress, we asked whether muropeptides accumulate and function in Mt to support host functions. We thus employed an established method to label PG with fluorescein isothiocyanate isomer I (FITC) and then examined PG muropeptide distribution in C. elegans. We first showed that the FITC-labeled PG (FITC-PG) is functional, as it suppresses the developmental delay phenotype in worms fed the erfK E. coli (FIG. 4A). Microscopy of animals fed with MitoTracker dye and FITC-PG revealed strong FITC-muropeptide signals in the intestinal cells (FIG. 4B) and FITC-PG puncta in intestinal lumen (FIG. 14). We then isolated Mt from FITC-PG fed animals and found that 34% of isolated Mt were labeled with both FITC-muropeptides and MitoTracker dye (FIG. 4C), supporting that muropeptides reside in Mt. FITC negative Mt (66%) were likely isolated from non-intestinal tissues that may not absorb FITC-muropeptides (FIG. 4C).

We further examined the muropeptide level in worm Mt by employing an established HEK-Blue-NOD1 assay (InvivoGen), and found that Mt lysates from worms induced NOD1 activity (FIG. 4D), providing further evidence that muropeptides accumulate in C. elegans Mt. In addition, the activity in the Mt lysates from worms fed wild-type E. coli was significantly higher than that from worms fed erfK E. coli (FIG. 4D), indicating the Mt accumulation of muropeptides is significantly reduced in C. elegans fed the mutant E. coli.

### Example 7: Muropeptides Interact with ATP Synthase for the Beneficial Functions

To gain more insight into the mechanism underlying the beneficial effects of muropeptides on host animals, we performed PG pull down and mass spectrometric (MS) analysis to identify PG-binding proteins in C. elegans. Using a relatively stringent cut-off point of score at 35, we identified 168 peptides as potential protein interactors with PG (Table 2). If the interaction of PG molecules with a particular protein is critically involved in the beneficial impact of the muropeptides on animal physiology, then RNAi knockdown of the candidate may generate phenotypes similar to that caused by the PG mutant diet. We thus performed RNAi analysis of all 168 candidate genes and found RNAi of 6 of them, including 5 that encode Mt proteins, significantly increased Phsp-6::gfp expression, developmental delay and food avoidance behavior (FIGS. 5A-5D, 15A and 15B). Interestingly, 4 of these 5 Mt proteins are subunits of the F1 complex of ATP synthase (atp-1, atp-2, atp-4, atp-5) (FIG. 15A).

We then carried out three additional tests to analyze the interaction between PG muropeptides and ATP synthase. First, we incubated isolated PG from wild-type E. coli with worm lysate and found that the PG molecules efficiently bound to ATP-1 in a concentration dependent manner (FIG. 5E). Second, we treated PG with Protease-K and found that Protease-K treated PG can no longer pulldown ATP synthase (FIG. 5F), which suggests that the short amino acid peptides associated with muropeptides are necessary not only for the functions, as shown earlier (FIGS. 3B-3D, ProK-PG), but also for binding to ATP synthase. Finally, we found that the step 3 product of PG purification (prior to trypsin treatment) failed to pull down ATP-1 (FIG. 5G), consistent with the notion that presence of lipoprotein compromises the effect of muropeptides (FIGS. 13B-D, erfK and ygeR). Collectively, these results indicate that the ability of PG muropeptides to bind ATP synthase is tightly correlated with the ability to provide the beneficial function in C. elegans.

We further tested whether the ATP synthase impacts muropeptides distribution and function. Since atp-1 (RNAi) causes first generation developmental arrest and F1 cannot be tested, we knocked down atp-4, which partially induces the phenotypes in the F1 generation. First, we showed that knockdown of atp-4 significantly reduced the level of intestinal FITC-muropeptides (FIG. 5H), indicating that ATP-4/ATP synthase is necessary for the accumulation of muropeptides in the intestine. Second, while adding isolated PG to worms fed erfK E. coli could suppress the phenotypes (Mt stress, developmental delay and food avoidance), this suppression effect was eliminated when the PG was added to atp-4 (RNAi)-treated worms (FIGS. 51-5K). Together, these data revealed a mechanism by which the muropeptides-ATP synthase interaction plays a critical role in mediating the beneficial effect of PG on the host physiology.

### Example 8: Muropeptides Display Antagonistic Interaction with a Known ATP Synthase Inhibitor

Previous studies have shown the profound impact of inhibiting ATP synthase activity on Mt ROS production, cellular metabolism, aging and cancer cell progression. The ATPase Inhibitory Factor 1 (IF1) is a known physiological inhibitor of ATP synthase that acts through binding to the F1 domain of the enzyme, and has been shown to play important roles in both mammalian cells and C. elegans under stress conditions and in tumor cells. The C. elegans genome encodes two IF1 homologs, MAI-1 and MAI-2, but only MAI-2 contains the Mt targeting sequence (MTS). We thus tested the functional relationship between muropeptides and MAI-2 by measuring the growth rate, UPR^{mt} and food avoidance behavior in a mai-2 loss-of-function (−) mutant, mai-2 (xm19). Indeed, mai-2 (−) partially alleviated all the phenotypes in worms fed erfK mutant E. coli (FIGS. 5L-5N and 15C and 15D). In addition, RNAi analysis confirmed that mai-1 is not critically involved in the process (FIG. 15E). This antagonistic relationship between muropeptides and a known inhibitor of ATP synthase provides further support for the role of muropeptides as an ATP synthase agonist.

### Example 9: Muropeptides Also Act as an ATP Synthase Agonist in Mammalian Intestinal Epithelial Cells

We next tested if the beneficial role of bacterial muropeptides on Mt health is conserved in mammals. By the HEK-Blue-NOD1 assay, we confirmed the presence of muropeptides in Mt of intestinal epithelial cells (IECs) isolated from live mice (FIG. 6). We then found that the a subunit of ATP synthase, ATP5A1, was also detected in our PG pull-down assay using caco-2 cells, a cell line derived from human colon cancer that morphologically and functionally resembles enterocytes (FIG. 7A). Consistent with our finding in C. elegans, the presence of the short peptides on PG is required for the ATP5A1-PG interaction (FIG. 7B). Moreover, our in vitro binding assay indicated that PG muropeptides directly interact with α, d and F, but not β, subunits of ATP synthase from both C. elegans and human cells (FIGS. 7C and 7D, 16C and 16D).

Next we investigated the molecular mechanism through which muropeptides regulate mammalian ATP synthase activity. First, we visualized the intact ATP synthase complex (complex V) by using non-denaturing blue native (BN)-PAGE electrophoresis. We found that intact complex V was increased by adding muropeptides to soluble mitochondria (FIG. 7E, 16A-B). This effect may not be due to a significant increase in the expression of ATP synthase subunits (FIG. 16E). Second, we performed a complex V activity assay (Abcam) to evaluate the impact of muropeptides on the activity of ATP synthase. The addition of muropeptides caused an increase in the activity of mammalian complex V in a concentration dependent manner (FIG. 7F), indicating that bacterial muropeptides act as an ATP synthase agonist. Moreover, we analyzed the Mt oxidative respiration of caco-2 cells by measuring the oxygen consumption rate (OCR), and observed that muropeptide supplementation caused a significant increase in both the basal and ATP-linked Mt oxidative respiration of caco-2 cells (FIGS. 7G and 7H). With the use of the ATPase inhibitor oligomycin and other chemicals, we determined that the majority of the increase in OCR was ATP-linked (FIGS. 7G and 7H). Consistent with this result, we also found that muropeptide supplementation boosted the ATP level and Mt membrane potential (Δψₘ) in caco-2 cells (FIGS. 7I and 7J). Furthermore, consistent with our finding in C. elegans (Figure. 2H and 12G), muropeptide supplementation also inhibited ROS production in caco-2 cells (FIG. 7K), supporting a conserved function of muropeptides. Altogether, these data support that muropeptides are an ATP synthase agonist that significantly impacts ATP production and oxidative respiration in Mt of mammalian cells.

### Example 10: Muropeptides Recover the Mitochondrial Function in ATP Synthase Deficiency Cells from a Leigh Syndrome Patient

Genetic mutations in subunits of ATP synthases are causal of more than 20 different neurological and metabolic diseases (Dautant et al., 2018). Since muropeptides stabilized the mammalian ATP synthase complex and promoted the activity (FIG. 7E-K, 16A-B), we hypothesized that muropeptides may also benefit mitochondria with ATP synthase deficiency, especially the mutations that affect complex V stability. To test this hypothesis, we decided to use the mutant primary fibroblast cells (GM13411) which were isolated from a Leigh syndrome patient (Coriell Institute). GM13411 (called m.8993T>G cells hereafter) contains a m.8993T>G mutation in the ATPase 6 gene, which causes complex V disassembly and may also cause proton translocation blockage, resulting in severe mitochondrial dysfunction, clinical symptoms and early death (Henriques et al., 2012; Pastores et al., 1994).

By comparison with wild-type control cells, we first verified that m.8993T>G cells exhibit severe mitochondrial defects (FIG. 17A-17D). Then we supplied muropeptides to m.8993T>G cells. and found that the Mt oxidative respiration was significantly restored (FIG. 8A-8D). Consistent with this result, we also observed that muropeptide addition boosted the ATP level and Δψₘ in m.8993T>G cells (FIGS. 8E and 8F). Studies have shown that the m.8993T>G mutation resulted in ROS overproduction and removal of ROS partially rescued ATP synthesis in the mutant cells (Baracca et al., 2007; Mattiazzi et al., 2004). Consistent with our finding in both C. elegans (Figure. 2H and 12G) and wild-type mammalian cells (FIG. 7K), muropeptide supplementation also inhibited ROS production in m.8993T>G cells (FIG. 8G). Overall, these results indicate that muropeptide supplementation is sufficient to recover the mitochondrial function significantly in ATP synthase deficiency cells.

### Example 11: Muropeptides Benefit the Fitness of ATP Synthase Deficiency Cells

We next investigated whether muropeptides improve the fitness of m.8993T>G cells. Among the m.8993T>G patients, elevated blood levels of creatine kinase, lactate and ammonia are common symptoms due to increased glycolysis (Henriques et al., 2012; Pastores et al., 1994; Rogatzki et al., 2015). Interestingly, our result showed that muropeptide supplementation significantly reduced the levels of all three molecules in culture medium (FIG. 9A-C), suggesting that muropeptides may also recover the metabolism and fitness of mutant cells. In line with this idea, we also found that the cell viability in galactose medium was partially rescued by muropeptides (FIG. 9D). All the results above support that muropeptides rescue the mutant ATP synthase function and improve the fitness of ATP synthase deficiency cells.

### Example 12: Muropeptides Recover Mitochondrial Structure and Suppress Mitochondrial Stress in m. 8993T>G Cells

Using electron microscopy, we analyzed the structure of mitochondria of m.8993T>G cells from a Leigh syndrome patient with and without the treatment of PG muropeptides. Most of the mitochondria from m.8993T>G cells lacked cristae that commonly present in wild type mitochondria (FIGS. 18A and 18B). Muropeptide supplementation effectively recovered the cristae structure in the mutant mitochondria in a concentration dependent manner (FIGS. 18A and 18B).

Mitochondrial stress and dysfunction are known to disrupt the structure and function of lysosomes and thus cause an increase in the accumulation of vacuoles and endo/autophagosomes (Demers-Lamarche et al., 2016; Lee et al., 2012; Palikaras et al., 2018). Indeed, high accumulation of vacuoles and endo/autophagosomes are high in m.8993T>G cells, and such an increase is partially suppressed by muropeptide supplementation (FIG. 19A-D). Consistent with that, the level autophagy marker LC3-II was increased in m.8993T>G cells and drastically reduced by the addition of PG muropeptides (FIG. 19G-H). In addition, we also show that muropeptide addition drastically reduced the expression of hsp60 that is commonly used as a marker for stress (FIGS. 19E and 19F).

### Example 13: PG Muropeptides Suppress Oxidative Stress in the Small Intestine of Antibiotic-Treated Mice

Antibiotic treatment kills or inhibits the growth of gut bacterial and is known to cause an increase of ROS production and oxidative stress (Guillouzo and Guguen-Guillouzo, 2020; Kalghatgi et al., 2013). We thus tested if oral supplementation of PG could reduce ROS production in antibiotic-treated mice. As indicated in FIG. 20, treating mice with antibiotics drastically reduced the bacterial level in fecal samples and increased the size of cecum and spleen. Supplementation with PG by oral gavage partially decreased cecum weight in male mice and increased spleen weight in female mice. Significantly, antibiotic treatment also caused a drastic increase in the ROS production in mice, and such an increase was mostly suppressed by PG supplementation (FIGS. 21A and 21B). In addition, PG also significantly suppressed the mitochondrial dysfunction-induced changes in energy metabolism (increase in the levels of glycolytic proteins PKM2, pyruvate dehydrogenase, and GAPDH) (FIGS. 21C and D).

### Example 14, Muropeptides Promote ATP Production and Survival of Cells of Two Alzheimer's Disease (AD) Models

As an early step to evaluate the potential of using PG muropeptides to treatment neurodegenerative diseases, we analyzed the effect of PG muropeptides on ATP production and cell survival of cells of familial type 1 and type 3 models for AD (obtained from Coriell Institute) (Iannuzzi et al., 2021; Nee et al., 1983). We observed that adding PG muropeptides led to significant increases in ATP production in cells of familial type 1 and type 3 AD models (FIG. 22A). Moreover, PG muropeptide supplementation significantly increased the cell survival rate of both cell types (FIGS. 22B and 22C).

### Example 15. PG Muropeptides Promote Cell Survival in Friedreich's Ataxia (FRDA) Cell Model

Friedreich's Ataxia (FRDA) is a severe neurodegenerative human disease caused by a mutation in the nuclear-encoded FXN gene. FXN encodes the Frataxin protein, a small mitochondrial protein that plays an essential role in iron-sulfur biogenesis. Mutations in FXN result in impaired mitochondrial function that contributes to loss of motor control, diabetes, and cardiomyopathy. We tested if supplementation with PG muropeptide would benefit survival of patient-derived fibroblasts (Coriell Institute) under an established stress condition (Jauslin et al., 2003; Jauslin et al., 2002). We found that supplementation with PG muropeptide promoted cell survival under chemical-induced stress (glutathione inhibitor, BSO) (FIG. 24).

### Example 16. PG Muropeptides Promote Cell Proliferation in a Rett Syndrome Cell Model

Rett Syndrome is a rare genetic neurological and developmental disorder that is caused by mutations in the X-linked gene MECP2. These patients display autistic behaviors, intellectual disability, and epilepsy. On the cellular level, Mt defects in morphology, increased ROS, and decreased ATP levels have been observed in mouse models (Ortiz-Gonzalez, 2021). Mutations in MECP2 dysregulate Uqcrc1 (Mt complex III subunit), COX1 (Mt complex IV subunit), and other Mt factors. We tested if supplementation with PG muropeptide would benefit proliferation of patient-derived fibroblasts (Coriell Institute). We found that Rett Syndrome (MECP2) fibroblasts had decreased cell proliferation compared to an age-matched WT control, and supplementation with PG muropeptide promoted cell proliferation in the mutant cells (FIG. 25A).

### Example 17. PG Muropeptides Promote Cell Proliferation in a Fragile X Syndrome (FXS) Cell Model

Fragile X Syndrome (FXS) is caused by mutations in the X-linked gene FMR1, and results in decreased expression of the protein FMRP that leads to a pathological mitochondrial proton leak (ATP-synthase c-subunit). FMRP is an RNA binding protein that acts as a repressor of translation, and has been implicated in many neuropsychological disorders (LaFauci et al., 2016). FXS is one of the most common causes of syndromic intellectual disability and autism (Ortiz-Gonzalez, 2021). We tested if supplementation with PG muropeptide would benefit proliferation of patient-derived fibroblasts (Coriell Institute). We found that supplementation with PG muropeptide promoted cell proliferation in Fragile X Syndrome (FMR1) fibroblasts (FIG. 25B).

### Example 18. PG Muropeptides Promote Cell Proliferation in Fibroblasts from Young and Old Apparently Healthy Individuals

Many cellular functions decline with age, including mitochondrial function. Human primary fibroblasts have been reported to have decreased mitochondrial respiration and ATP production as a function of age (Schniertshauer et al., 2018). We tested if supplementation with PG muropeptide would benefit proliferation of fibroblasts derived from both young and old apparently healthy human subjects (Coriell Institute). We found that supplementation with PG muropeptide promoted cell proliferation in fibroblasts derived from a young subject (1 yr) (FIG. 25C). Compared to a young sample (FIG. 25D, gray bar), the fibroblasts derived from an older subject had reduced cell proliferation (black bar) that was increased with PG muropeptide supplementation in a dose dependent manner (white bars).

### Example 19. Rationales for the High Potential of Using PG Muropeptides to Treat Many Mitochondria Dysfunction Related Diseases or Health Conditions

As described above, the use of PG muropeptides include broad applicability to a variety of mitochondrial diseases as described herein. As further supported by references 138-180 provided below, the physiological effect of PG muropeptides can be predictably applied to a number of known mitochondrial diseases in mammals, and in particular humans. For example, the therapeutic application of PG muropeptides may be applied to the following mitochondrial diseases:

### 1. Genetic Mitochondria (Mt) Dysfunction Syndromes

More than 1 of 5000 newborn humans are affected by over 150 genetic Mt dysfunction syndromes (Dautant et al., 2018; Skladal et al., 2003). These diseases, caused by mutations in genes encoding proteins playing critical roles in Mt functions and biogenesis, are characterized by a drastically reduced production of ATP by oxidative phosphorylation and commonly associated with increase in ROS and oxidative stress (Dautant et al., 2018; Stewart and Chinnery, 2015). These genetic Mt dysfunction syndromes share some common pathological defects including visual/hearing weakness, encephalopathies, cardiomyopathies, myopathies, diabetes, liver and renal dysfunctions. The causal-effect relationship between Mt dysfunction and clinical traits of these diseases are well defined by the functional analyses of these genetic mutations (Dautant 2019) (Lott et al., 2013).

On a specific note, 15% of these genetic mutations (over 600 different point mutations) are in Mt DNA (mtDNA) including mtDNA encoding subunits of ATP synthases (Dautant et al., 2018). Mutations in mtDNA encoding subunits 8 and a of ATP synthases alone cause more than 20 different type of diseases: Apical hypertrophic cardiomyopathy (AHCM) and neuropathy, ataxia, autism, Charcot-Marie-Tooth syndrome (CMT), encephalopathy, epilepsy with brain pseudoatrophy, Episodic Weakness, Hereditary Spastic Paraplegia (HSP), Familiar Bilateral Striatal Necrosis (FBSN), Infantile cardiomyopathy, Leber Hereditary Optic neuropathy (LHON), Left Ventricular Hyper Trabeculation syndrome (LVHT), Maternally inherited Diabetes and Deafness syndrome (MIDD), Maternally inherited Leigh Syndrome (MILS), Mesial Temporal Lobe Epilepsies with Hippocampal Sclerosis (MTLE-HS), Metabolic Syndrome (MS), Motor Neuron Syndrome (MNS), Myopathy, lactic Acidosis, and Sideroblastic Anemia (MLASA), Neurogenetic Ataxia Retinis Pigmentosa syndrome (NARP), Periodic paralyzes, Schizophrenia, Spino Cerebellar Ataxia (SCA) and Tetralogy of Fallot (ToF).

Other genetic mitochondria dysfunction disorders also include many well-defined diseases: short-chain, medium-chain and long-chain acyl-coA dehydrogenase deficiency (SCAD, MCAD and LCAD), chronic progressive ophthalmoplegia (CPEO), Pearson Syndrome, Barth Syndrome, Alpers Disease, Luft Disease, mitochondrial neurogastrointestinal encephalomyopathy (MNGIE), neuropathy, ataxia, and retinal pigmentosa (NARP), myoclonic epilepsy and ragged red fibers (MERRF), mitochondrial encephalopathy, and lactic acidosis, and stroke-like episodes (MELAS).

The therapeutic application of PG muropeptide to treat these genetic Mt dysfunction syndromes has been demonstrated by the present inventors data showing that PG muropeptides act as agonists of ATP Synthase to promote Mt functions and biogenesis, and by showing that adding muropeptides to cells from patients of Leigh Syndrome is highly effective in recovering Mt structure/functions and suppressing oxidative stress (FIGS. 8-9; 18-19).

### 2. Neurodegenerative Diseases

It is well known that Mt dysfunction and oxidative stress are commonly associated with age related neurodegenerative diseases including Alzheimer's diseases (AD), Parkinson's disease (PD), Huntington's disease (HD) and amyotrophic lateral sclerosis (ALS, aka Lou Gehring's diseases) (Johnson et al., 2021; Lin and Beal, 2006). Despite heterogenicity in clinical traits in these diseases, past extensive research results have clearly indicated that Mt dysfunction and oxidative stress play critical roles in the pathogenesis of these diseases (Lin, 2006 #539) (Johnson et al., 2021).

In case of AD, oxidative damage including apoptosis occurs early in the brain prior to the deposit of Ab and the onset of significant plaque pathology, and energy deficiency is a fundamental characteristic feature of both AD brains and peripheral cells (Beal, 2005; Nunomura et al., 2001; Reddy et al., 2012). On the other hand, Mt dysfunction and oxidative stress are critically involved in Ab induced apoptosis of neurons. Studies in various animal models have indicated that increase in Mt oxidative stress such as by reducing the activity of Mt SOD enzyme, treating animals with hydrogen peroxide, or replacing mouse NOS2 gene with human NOS2, has led to increase brain Ab level and plaque deposition, learning and memory deficits, neuronal loss and/or neurofibrillary tangle pathologies (Johnson et al., 2021; Vitek et al., 2020). Mechanistically, oxidative stress alters the activity of key regulators or signaling pathways, such as activate JNK and p38 stress pathways and Pin1 enzyme that are known to play critical roles in pathogenesis of AD (Johnson et al., 2021; Lin and Beal, 2006). Moreover, several key regulators of Mt biogenesis and homeostasis, such as PGC1a that regulates Mt biogenesis, are significantly reduced in AD animal models, and artificially elevate PGC1a reverses Mt dysfunction, reduces neuronal loss, and improves spatial and recognition memory in cellular and/or mouse models of AD (Johnson et al., 2021; Lin and Beal, 2006). Knowing the causal effects of Mt dysfunction on AD pathogenesis, extensive efforts have been made in testing the potential of various antioxidants in treating AD (Pritam et al., 2022).

For PD, the critical role Mt dysfunction and oxidative stress on pathogenesis of AD are also well indicated in extensive past studies (Surmeier et al., 2017). Defects in Mt ETC complexes are found in many substantia nigra neurons in PD patients, and in some studies, defects in the complexes were found in infant patient prior to loss of neurons (Surmeier et al., 2017; Tzoulis et al., 2014). Mutations in several PARK genes that are involved in maintaining cause early-onset forms of PD and disrupting complex I in dopaminergic neurons specifically cause defects mimicking progressive PD (Beilina and Cookson, 2016; Gonzalez-Rodriguez et al., 2021). Moreover, mutations in multiple genes associated with autosomal dominant PD (PINK1, Parkin, Dj-1) are all linked to critical Mt dysfunctions (Haelterman et al., 2014).

For HD, impairment of Mt respiration (ETC activity) and ATP production have been observed in HD brain as well as in stratal cells from HD mouse model (Milakovic and Johnson, 2005) (Lin and Beal, 2006; Mochel and Haller, 2011). Inhibiting factors in the ETC using Mt toxins has caused clinical defects similar to that of HD (Brouillet et al., 1995). Conversely, promoting ETC activity by overexpressing complex II proteins suppresses Mt dysfunction and cell death (Benchoua et al., 2006).

These research results argue that ameliorating Mt dysfunction and suppressing oxidative stress is a predictable mechanism to these diseases. Indeed, many chemical drugs, which reduce Mt oxidative stress or enhance Mt function (MitoQ, Co-enzyme Q10, vitamin B3, NAC, UDCA, NAD+, and creatine), were tested in animal models for therapeutical usage in treating these diseases including AD, PD and HD (Cenini and Voos, 2019; Mochel and Haller, 2011; Prasuhn et al., 2020; Pritam et al., 2022). Some of them, such as MitoQ, NAC and NAD+ have been tested in clinical trials for AD (Cenini and Voos, 2019; Pritam et al., 2022). Our finding that PG muropeptides function as agonists of ATP synthase (complex V) and ETC indicates the unique therapeutic potential in this regard. PG muropeptides are not simply antioxidants; they suppress ROS production and oxidative stress by promoting ETC activity and ATP production that in turn facilitate Mt biogenesis and structural integrity. These effects are seen in cultured cells and animal models that do not contain genetic mutations that directly impaired ETC or ATP synthase, as well as in cells from patients with genetic Mt dysfunction syndrome (Leigh syndrome) and cells for familiar type 1 and type 3 AD model (FIGS. 8-9;18,19 and 22).

### 3. Other Neurological Diseases.

Mt dysfunction has also been tightly linked to many other well-known neurological disorders such as Epilepsy, Autism, Fibromyalgia, chronic fatigue, cerebral palsy, Friedreich's Ataxia, Rett Syndrome, and Fragile X Syndrome (Finsterer and Zarrouk Mahjoub, 2012; Grace et al., 2016; Mahalaxmi et al., 2021; Ortiz-Gonzalez, 2021; Rahman, 2012; Tragni et al., 2022). For example, many of genetic Mt dysfunction syndromes (including Leigh syndrome) mentioned earlier display clinical traits of these diseases. Accumulated data also suggest that promoting ETC function and release oxidative stress reduces pathological damages to patients of many of these diseases as our data on Leigh syndrome cells implicated

As for Epilepsy, failure in early steps in ETC is known to be causal to pathological defects in the patients. (Finsterer and Zarrouk Mahjoub, 2012; Rahman, 2012). It is also known that ROS production from seizures leads to damage to Mt DNA, which in turn reduces the ETC activity and further increase oxidative stress.

As for Autism, collective data from various analyses of autism patients also uncovered abnormality in ETC and Mt function in a large percentage of autism patients, and Mt dysfunction may be associated with neurodevelopmental repression (NDR) commonly seen in Autism patients (Frye, 2020; Mahalaxmi et al., 2021; Valiente-Palleja et al., 2018).

As for Fibromyalgia (FM) and chronic fatigue (CF), the impact of Mt dysfunction on these conditions were suggested by observed increase in ROS and oxidative stress, decrease in Mt mass and coenzyme Q10 (Sawaddiruk et al., 2019), as well as the causal relationship between ROS/oxidative stress on chronic pain, inflammatory proteins, and reactive lipid peroxidation that all known to cause peripheral and central sensitization and hyperalgesia (Grace et al., 2016; Sawaddiruk et al., 2019).

The impacts of Mt dysfunction on these diverse neurological diseases are also the basis for antioxidants being commonly used to treat these diseases or test therapeutic potential in animal models. For example, antioxidant cocktails being are routinely administrated to Epilepsy patients (Tragni et al., 2022; Yang et al., 2020). Co enzyme Q10, Vitamin D, NAC and others have been tested to treat autism with some positive responses (Ortiz-Gonzalez, 2021; Sawaddiruk et al., 2019). Coenzyme Q10 has been tested in clinical trial for alleviating pain in pregablin-treated fibromyalgia patients via reducing brain activity and Mt dysfunction. (Sawaddiruk et al., 2019). Our discovery of the unique roles of PG muropeptides in increasing the activity of ATP synthase and ETC, as well as suppressing Mt oxidative stress, indicate muropeptides may act much more effectively than simple antioxidants in treating some, if not most, of these neurological conditions. We have observed that addition of muropeptides significantly improved cell proliferation and survival of fibroblast cells derived from patients of Friedreich's Ataxia, Rett Syndrome, and Fragile X Syndrome (FIG. 24-25).

### 4. Cardiomyopathy and Muscular Dystrophy (MD)

Myopathy is also one of the genetic Mt dysfunction diseases (e.g., mutation in ETC complex I and ATP Synthase (Dautant et al., 2018; Fassone et al., 2011), but the evidence for the impact of Mt dysfunction on heat failure and muscle degeneration are indicated by abundant data beyond genetic mutations in Mt genes. For cardiomyopathy, it has been known that heart failure is associated with insufficient energy production (Energy starvation hypothesis) (Zhou and Tian, 2018). ROS-induced damage has been known as a major pathogenic mechanism, as excessive ROS is seen in human patients and animal models and Mt-targeted ROS scavenging has been shown to alleviate the defects in animal models of heart failure (Zhou and Tian, 2018). ROS and oxidative stress are known to impact metabolic activity of Mt by oxidative modification of proteins and lipids that disrupt their normal functions including inhibiting ATP synthase (Zhou and Tian, 2018). Similarly, decrease in Mt respiration and ATP production is also seen in MD patients and mouse models.

The potential of suppressing Mt oxidative stress in treating cardiomyopathy and MD is also indicated by that ROS-scavenging compounds have also been tested in clinical trial for treating cardiomyopathy patients (e.g., NCT03506633, NCT02966665, NCT01925937, ClinicalTrials.gov). It is important to point out, previous studies found that ATP synthase activity is inhibited in the failing heart because of increased protein acetylation (Boudina et al., 2007; Chouchani et al., 2014). Since it is well known that inhibiting ATP synthase causses impairment of ETC flow, increase of ROS production and inducing apoptosis, promoting ATP synthase activity can be effective in treating cardiomyopathy, which has been shown in PG muropeptides' new function.

### 5. Metabolic Diseases.

Given the critical role of Mt in energy metabolism, it's close relationship with metabolic disorders is predictable. Indeed, decline in Mt functions are seen in patients or mouse models of major metabolic disorders such as diabetics (James et al., 2012; Prasun, 2020). Although studies have shown that insulin resistance are likely causal, rather than consequential, to Mt decline, the latter exacerbates insulin resistance and other pathological traits in diabetic conditions (Montgomery and Turner, 2015; Prasun, 2020). More specifically, increased ROS oxidative stress would further damage Mt function by oxidative modification of key components of ETC and other Mt components, leading to Mt fragmentation and further decrease in oxidative phosphorylation. Increased ROS may also induce the release of inflammatory factors that will enhance the problem of insulin resistance. The impact of ROS in pancreatic beta cells will have more direct impact on insulin production. Moreover, increase in Mt ROS may cause undesired metabolic change and inhibit b oxidation of fatty acids that are all known to be associated with type II diabetics (James et al., 2012). Therefore, despite that Mt dysfunction may not be the primary cause for many metabolic disorders, promoting Mt biosynthesis and function, as well as enhancing antioxidative capacity are still considered to be viable therapeutic strategies further validating the therapeutic role of PG muropeptides in treat metabolic diseases. (Prasun, 2020).

### 6. Suppressing Side Effects of Antibiotics Treatment of Bacterial Infection.

With their ability to kill or inhibit growth of bacteria, antibiotics are routinely used to treat bacterial infections in humans and domestic animals. However, antibiotics treatment is also well known for deleterious side effects. More specifically, antibiotics are known to induce mitochondrial dysfunction and oxidative stress that leads to damage to DNA, proteins and lipids in cultured mammalian cells and mice (Guillouzo and Guguen-Guillouzo, 2020; Kalghatgi et al., 2013). Elevated oxidative stress leads to damage to DNA proteins and lipids. Interactions between antibiotics and proteins in mitochondria have been shown to cause oxidative stress in mammalian cells or in mouse model (Guillouzo and Guguen-Guillouzo, 2020; Lowes et al., 2009; McKee et al., 2006). In addition, based on our finding in C. elegans described in this patent application, lack of PG muropeptides released from commensal bacteria in antibiotics-treated mammals might also contribute to an increase in oxidative stress. Antioxidant treatment has been shown to be effective in alleviating the oxidative stress related negative effects of bactericidal antibiotics (Kalghatgi et al., 2013). In this application, we provide data that feeding mice with PG muropeptides suppressed ROS production and mitochondrial dysfunction-induced change in energy metabolism in intestinal epithelial cells in mice treated with multiple antibiotics to kill commensal bacteria (FIG. 21). This result indicates that muropeptides may be used to reduce oxidative stress caused by antibiotics-induced mitochondrial dysfunction.

### Example 20: Materials and Methods

C. elegans strains and maintenance: C. elegans strains were grown and maintained on nematode growth media (NGM) plates seeded with the Escherichia coli strain OP50 at 20° C. The following strains were obtained from Caenorhabditis Genetics Center (CGC): N2 Bristol (wild-type control strain), SJ4100: zcIs13 [Phsp-6::gfp] V, SJ4058: zcIs9 [Phsp-60::gfp+lin-15 (+)/V, SJ4197: zcIs39 [Pdve-1::dve-1::gfp] II, SJ4005: zcIs4 [Phsp-4::gfp] V, TJ375: gpIs1 [Phsp-16.2::gfp], AY101: acIs101 [PF35E12.5::gfp+rol-6(su1006)], AU133: agIs17 [Pmyo-2::mCherry+Pirg-1::gfp] IV, CF1553: muIs84 [(pAD76) sod-3p::GFP+rol-6(su1006)]. mai-2 (xm19) was obtained from Dr. R. E. Navarro (Universidad Nacional Autonoma de Mexico).

E. coli Keio collection screen: Bacterial peptidoglycan (PG) metabolism-related genes were determined based on the published papers (Table 1) and the PG mutant sub-library was assembled from the Keio E. coli single mutant collection. Mutant bacteria strains, as well as the wild-type control strain BW25113, were cultured overnight in LB medium with 50 μg/ml kanamycin in 96-well plates at 37° C. 100 μl of the overnight E. coli cultures were spotted onto 6 cm NGM plates and dried at room temperature for 1h before use. About 200 synchronized L1 worms were then placed on the seeded NGM plates and cultured at 20° C. Animals were screened after 54-56 hours. Worm developmental rates were determined by vulval development as previously described. To quantify the developmental rate, about 50 animals were scored under Nomarski microscope. To verify the genotypes of the 4 candidate Keio mutants, PCR were performed on each mutant colony and the control BW25113 strain using specific downstream genomic primers together with kanamycin-cassette-specific primers.

Food avoidance behavior assay: Food avoidance behavior assay was determined using a method adapted from a published procedure (Kniazeva et al., 2015; Melo and Ruvkun, 2012). Briefly, overnight cultures of wild type and PG mutant E. coli were adjusted to the same OD₆₀₀ and 20 μl of each culture was seeded on the center of 6 cm NGM plates. The seeded plates were dried at room temperature for 1 hour before use. About 100 synchronized L1 animals were then dropped onto the bacterial lawns and cultured for 45 hours at 20° C. The avoidance index was determined by N_{off/on}/Nₜₒₜₐₗ. Each assay was conducted in triplicate.

Heat-killed E. coli preparation and supplementation: We followed an established protocol to prepare heat-killed (HK) E. coli: standard overnight bacterial cultures were concentrated to 1/10 vol and were then heated in a 75° C. water bath for 90 min. For HK-E. coli supplementation, live E. coli and HK-E. coli were mixed at a 1:1 ratio then 20 μl (avoidance behavior assay) or 50 μl (other assays) mixtures were dropped on the NGM plates and dried at room temperature for 10 min before use. About 100-200 synchronized L1 animals were placed on the bacterial lawn to grow at 20° C.

PG isolation, enzyme treatment and supplementation assays: An adapted method was used to isolate peptidoglycan from bacterial cultures. In brief, bacterial pellets were resuspended in 1/10 vol 1M NaCl solution and boiled at 100° C. in a heating block for 1 h. After washing 4 times with distilled water, the insoluble cell wall preparations were incubated in an ultrasonic water bath (Laboratory Supplies Co., INC, G112SPIT) for 1h, followed by stepwise digestion using DNAse/RNAse and trypsin at 37° C. for 1h, respectively. The solutions were boiled for 5 min at 100° C. to inactive the enzymes, followed by washing twice with distilled water. The PG pellets were recovered by centrifugation at 13,000 rpm for 10 min and stored at 4° C. For further enzymatic treatment of isolated PG, lysozyme (Fisher 44-031-GM, 300 μg/ml), AmiD (purified from BL21 E. coli, 300 μg/ml) or NagZ (purified from BL21 E. coli, 300 μg/ml) were added to PG solution (20 mM HEPES pH7.5) and incubated at 37° C. for 48 h. The enzymatic reactions were stopped by incubating with 50 μg/ml trypsin at 37° C. for 2 h. The supernatants were collected after centrifugation at 12,000 rpm for 10 min. Muropeptides were collected from the supernatant of lysozyme-treated PG and stored at −20° C. for further assays. For Protease-K treatment, 400 μg/ml Protease-K was added to PG solution (20 mM HEPES pH7.5) and incubated at 37° C. for 2 h. The enzymatic reaction was stopped by boiling at 100° C. for 15 min. For supplementation assays, indicated volume of live E. coli and PG solution mixtures (1:1 ratio) were seeded on NGM plates and dried for 10 min. C. elegans feeding assays were performed as described above.

Mixed E. coli feeding assays: Overnight E. coli cultures were adjusted by OD₆₀₀. Single PG mutant cultures or double PG mutant culture mixtures (1:1 ratio) were seeded on NGM plates and dried for 10 min at room temperature. Then about 100 synchronized L1 animals were placed on the bacterial lawn to grow at 20° C.

Chemical supplementation: The NAC (Fisher Scientific, ICN10009825) stock solution (500 mM) was made in ddH2O. For NAC supplementation, NAC solution was added in NGM agar to the final concentration of 50 mM. For iron supplementation, FeCl₃ (Sigma, 236489) was dissolved in ddH2O to generate solutions of desired concentration (2 mM, 8 mM and 20 mM). The FeCl₃ solutions were mixed with live E. coli (1:1 ratio) and seeded on the NGM plates.

For tunicamycin treatment, tunicamycin (Fisher Scientific, ICN15002801) was mixed with live E. coli to a final concentration of 5 μg/ml and spotted on the NGM plates. C. elegans feeding assays were performed as described above.

Heat shock treatment of worms: 100 synchronized L1 larvae were grown to early L4 (24 h, 20° C.), then incubated at 35° C. for 1 h and allowed to recover overnight at 20° C. before microscopic examination.

FITC-PG labeling: An established protocol was modified to label PG with FITC (Mann et al., 2016). Briefly, PG was isolated as described above and then sonicated in a water-bath sonicator for 1 h. The sonicated PG was suspended in 500 μl FITC solution (Fisher, 71-190-0, 1 mg/ml in sterile carbonate buffer). The tubes containing the solutions were wrapped in aluminum foil to avoid exposure to light and shaken for 1 h at room temperature. The FITC-PG sample was washed more than 6 times to remove unlabeled FITC and resuspend in ddH2O. The FITC-PG feeding assays were performed as described in PG supplementation procedures. As negative controls, 10 μM MitoTracker-Red CMXRos and/or FITC dye were supplemented on live bacteria lawns. All the NGM plates were wrapped in aluminum foil to protect from light. L4 larvae animals were fixed in 4% formaldehyde with 6 mM K₂HPO₄ (PH 7.2) and 75% methanol for 10 min at −20° C. to remove auto-fluorescence in the intestine. The fixed worms were rinsed three times in PBS before microscopic examination.

Mitochondrial (Mt) extraction: Mt from worms were extracted from L4 larvae with the Mt Isolation Kit for Tissue (Thermo Scientific, 89801), following the manufacturer's protocols.

HEK-Blue-NOD1 assay: Isolated Mt were lysed with Tris-buffed saline (25 mM Tris-Hcl, pH 7.2, 150 mM NaCl, 1× protease inhibitor) and centrifuged at 12,000 g for 5 min to collect the supernatant. Samples were adjusted based on Mt total protein as determined by the BCA protein assay kit (Thermo Scientific, 23225). HEK293 cells expressing the human NOD1 receptor and the NF-κB SEAP reporter genes (Invitrogen, hkb-hnod1) were used according to the manufacturer's instructions to assess the muropeptides in mitochondrial lysates. In brief, 20 μL of adjusted Mt lysates, or lysis buffer (mock), were added to single well of a 96-well plate. A cell suspension (˜280,000 cells per ml in HEK Blue detection medium) was prepared and 180 μl was added to wells with mitochondrial lysates or mock lysate. The 96-well plate was incubated at 37° C. overnight and assessed by reading OD at 650 nm.

Isolation and identification of PG binding proteins: A modified method was used to identify the PG binding proteins from C. elegans lysate. In brief, ˜100 μl worm pellet was suspended in lysis buffer (100 mM NaCl, 0.5 mM EDTA, 0.5% NP-40 and a cocktail of protease inhibitors in 10 mM Tris-HCl, pH 7.4), followed by sonication. After centrifugation, the supernatant was mixed with about 200 ug isolated PG and incubated at 4° C. for 1 h. The PG binding proteins (PG-BPs) were collected by centrifuging at 4° C. at 13,000 g for 10 min. After washing 3 times with a wash buffer (150 mM NaCl in 10 mM Tris-HCl, pH 7.4), proteins that bound to PG were eluted by boiling at 100° C. for 15 min. The supernatant was collected by centrifuging at 13,000 g for 10 min and subjected to LC-MS analysis.

Protein expression and purification: The nagZ (1-1023 bp) and AmiD (52-831 bp) genes were PCR amplified from WT K-12 BW25113 genomic DNA. All gene constructs were inserted into a pET28a vector. These constructs were transformed into E. coli BL21 (DE3) cells and grown in LB broth at 37° C. overnight. The overnight cultures were diluted with 1/100 vol of fresh LB broth and cultured until OD₆₀₀ reached ˜0.6. The expression of recombinant proteins was induced by 0.1 mM IPTG at 20° C. for 19 h. Bacterial cells were harvested by centrifugation at 5000 g for 10 min at 4° C. After centrifugation, the pellets were lysed in buffer containing 25 mM HEPES pH 7.5, 500 mM NaCl, 0.5% NP-40, 1 mM DTT and 1× protease inhibitor, followed by centrifugation at 12,000 g for 30 min at 4° C. The supernatants were incubated with Ni-NTA agarose beads (Qiagen, 151028822) for 1 h and rinsed 3 times with a wash buffer (25 mM HEPES pH 7.5, 500 mM NaCl, 1 mM DTT and 30 mM imidazole). Proteins were eluted with buffer containing 25 mM HEPES pH 7.5, 500 mM NaCl, 1 mM DTT and 500 mM imidazole. Amicon Ultra centrifugal filters (10 kD) were used to concentrate the proteins and exchange buffer (25 mM HEPES pH 7.5, 250 mM NaCl and 1 mM DTT). These proteins were then stored at −80° C. with 10% glycerol.

Verification of the interaction between PG and ATP synthase α-subunit: To confirm the interaction between PG and ATP-1 (C. elegans) or ATP5A1 (mammals), PG pull-down assays were performed as described above. Total protein of worms or caco-2 cell lysates were used to incubate with PG, protease-K treated PG or PG3. PG. Western blots were performed to detect ATP-1 (C. elegans) or ATP5A1 (caco-2 cells) (Thermo Fisher 43-9800).

RNAi treatment in C. elegans: RNAi plates were prepared by adding IPTG to NGM agar to a final concentration of 1 mM. Overnight E. coli cultures (LB broth containing 100 μg/ml ampicillin) of specific RNAi strains and the control HT115 strain were seeded to RNAi feeding plates and cultured at room temperature for 2 days before use. Synchronized L1 animals were added to the RNAi E. coli seeded plates. For transgenerational RNAi knockdown of atp-4, we fed synchronized L1 animals with OP50 for the first 12 h and then transferred the worms to atp-4 RNAi or control plates. The gravid adults were bleached to collect synchronized F1 worms, which were then fed with PG mutant E. coli.

ROS measurements in C. elegans: The ROS staining protocol was modified from. Briefly, L4 stage animals were collected and washed three times with M9 buffer to remove the bacteria. Then the worms were transferred into the staining solution (H2DCFDA, 50 uM in M9 buffer) and incubated for 30 min at 20° C. After washing twice with M9 buffer, worms were subjected to microscopic examination.

Microscopy: Analysis of fluorescence was performed under Nomarski optics on a Zeisis Axioplan2 microscope with a Zeiss AxioCam MRm CCD camera. Plate phenotypes were observed using a Leica MZ16F dissecting microscope with a Hamamatsu C4742-95 CCD camera.

Quantification and Statistical analysis: ImageJ software was used for quantifying fluorescence intensity of various of reporters. L4 to young adults were randomly picked under dissection microscope and imaged by the Nomarski microscope. To determine the developmental stages, animals were cultured 54-56 h, with the exception that animals for NAC supplementation and mai-2(−) were cultured about 60-62 h, before microscopic evaluation. We used the Student's t-test to determine the significant differences as indicated in the figure legends.

Mice: Experiments were performed according to a protocol approved by the Institutional Animal Care and Use Committee (IACUC) in University of Colorado. C57BL/6J strain mice were purchased from the Jackson Laboratories and housed in a barrier facility. Animals were used between 6-10 weeks of age; both males and females were used as noted in figure legends.

Antibiotic-induced microbiome depletion: For antibiotic treatment, age matched mice were randomly assigned to experimental groups, 3-5 mice were housed in each cage. We followed an established protocol to perform antibiotic-induced microbiome depletion (AIMD) (Zarrinpar et al., 2018). Briefly, one group of mice were given oral gavage of antibiotic cocktail or antibiotic cocktail plus purified PG muropeptide (2 mg/mouse) every 12 h. The antibiotic cocktail contained ampicillin (100 mg/kg), vancomycin (50 mg/kg), metronidazole (100 mg/kg), neomycin (100 mg/kg) and amphotericin B (1 mg/kg). The other group of mice received water as control. The cocktail was freshly prepared every 24 h. AIMD was maintained for 23-27 days.

Stool culture measurement: To assess microbiome depletion by the AIMD method, fresh fecal samples were collected in Eppendorf tubes containing 1 ml sterile PBS two weeks after AIMD initiation. Samples were diluted 1:100 in sterile PBS and thoroughly vortexed. Equal concentrations of stool diluent from different groups were placed on LB agar plates and incubated for 2-3 days at 37° C.

Cecum and spleen weight: Mice were euthanized by carbon dioxide asphyxiation followed by cervical dislocation. Cecum and spleen were carefully dissected and weighed by a precision scale.

IEC cell isolation: A modified protocol (Chang et al., 2009) was used to isolate IEC cells from a C57BL/6 mouse. Briefly, animals were euthanized by carbon dioxide asphyxiation followed by cervical dislocation, then the small intestines were dissected. The small intestines (jejunum) were flushed with sterile PBS, opened longitudinally, and cut into 5-mm fragments. The epithelial integrity was disrupted by 1.5 mM dithiothreitol (DTT) treatment on a shaker for 45 min at 37° C. Liberated IEC cells were collected and separated by Percoll gradient (Sigma, P4937). The interface cells were then collected and used as IEC cells.

Measurement of ROS: The ROS staining protocol was modified from (Wu and Yotnda, 2011). Briefly, isolated mouse IEC cells or culture cells were washed three times with PBS. The cells were then transferred into the staining solution (H2DCFDA, 1 uM in MEM media, no FBS) and incubated at 37° C., 5% CO2 for 30 min. After washing 4 times with MEM media (no FBS), cells were subjected to microscopic examination.

Cell culture: The fibroblast cells were purchased from Coriell Institute (GM13411, AG08245, AG07601, GM04078, GM00967, GM05659, GM21921, GM04026). Fibroblast cell were used no more than 15 passages after receipt. Cells were maintained in Eagle's Minimum Essential Medium (EMEM) media (ATCC, 30-2003) supplemented with 15% FBS (Gibco, 26140-079), Penicillin (100 U/ml) and Streptomycin (100 μg/ml). The cells were maintained in a humidified atmosphere at 37° C., 5% CO2.

Blue native PAGE analysis of ATP synthase complex: Mitochondria were extracted from HEK293T cells by kit (ThermoFisher, 89874) or purchased from abcam (ab110338, bovine heart mitochondria) and solubilized by detergent and 1× mitochondrial buffer (abcam, ab 109907). After centrifugation, the supernatant was collected for mock or PG muropeptide treatment. 30 μl mock buffer (lysozyme solution) or PG muropeptide was mixed with 30 μl mitochondrial supernatant, respectively, and incubated on ice for 30 min. Then, each sample was analyzed by BN-PAGE gel to separate the mitochondrial complexes. After membrane transfer and blocking, the antibody against ATP5A1 was used to detect ATP synthase complex.

Complex V activity assay: Complex V activity assay was performed using the MitoTox™ Complex V OXPHOS Activity Assay Kit (Abcam, ab109907), following the manufacturer's protocol. Approximate 21 μg of mitochondria were added to each well. OD₃₄₀ values were monitored by a SpectraMax M5 Plate reader in kinetic mode at 30° C.

Measurement of oxygen consumption rates (OCR): OCR measurements were performed with a Seahorse XF-24 analyzer (Seahorse Bioscience). Caco-2 cells were seeded in Seahorse XF-24 cell culture microplates at 1.7×10⁵ cells per well in 250 μl DMEM media (Gibco, 11995040) supplemented with 10% heat-inactivated FBS and 1% non-essential amino acid, and then incubated at 37° C. and 5% CO₂ overnight. The cells were incubated with muropeptide solution (about 800 μg/ml, prepared from lysozyme treated PG) or lysozyme solution (mock) for 48 h. Cells were washed in Seahorse XF DMEM media (Agilent, 30119005, supplemented with 1 mM pyruvate, 10 mM glucose and 2 mM glutamine) and incubated at 37° C. with no CO₂ for 1 h before measurement. Cells were washed again with Seahorse XF DMEM media just before measuring. OCR was measured and analyzed by injecting 2.5 μM oligomycin, 1 μM FCCP and 1.25 μM ROT/AA sequentially following the manufacture's instruction.

Measurement of mitochondrial membrane potential: Caco-2 cells were seeded in 24-well plate (inserted with glass coverslips (Neuvitro, GG12PDL)) at 1×10⁴ cells/well. After overnight recovery, cells were treated with either the lysozyme solution (mock) or muropeptides (800 μg/ml) for 48 h. Culture media were changed every 24 h. MitoTracker-Red CMXRos was diluted into DMEM media (Gibco, 11995040) (without FBS) to a final concentration of 100 nM just before use. Caco-2 cells were cultured to 50-60% confluency. The cells were washed 3 times with DMEM media (without FBS) and 500 μl MitoTracker-Red CMXRos solution was added. The cell culture plates were then wrapped in foil and maintained at 37° C. and 5% CO₂ for 30 min. The cells were then washed 3 times with DMEM media (without FBS) before microscopic examination. ImageJ was used to analyze mean fluorescence for mitotracker dyes.

Measurement of ATP levels: Caco-2 cells were seeded in 96-well plates at 2×10⁴ cells/well. After overnight recovery, cells were treated with either a lysozyme solution (mock) or muropeptides (800 μg/ml) for 57 h. The culture media were changed every 24 h. ATP levels were measured using the Luminescent ATP Detection Assay Kit (Abcam, ab113849). Luminescence was monitored by a SpectraMax M5 Plate reader. The protein concentrations were measured by BCA Protein Assay Kit (Thermo Scientific, 23227). ATP levels were normalized to total protein content.

For fibroblast cells ATP measurement, cells were seeded in 96-well plates at 1×10⁴ cells/well. After overnight recovery, cells were treated with either a lysozyme solution (mock) or muropeptides (500 μg/ml) for 24 h. ATP levels were measured using the Luminescent ATP Detection Assay Kit (Abcam, ab113849). Luminescence was monitored by a SpectraMax M5 Plate reader. The protein concentrations were measured by BCA Protein Assay Kit (Thermo Scientific, 23227). ATP levels were normalized to total protein content.

Cell survival rate: Fibroblast cells were seeded in 24-well plates at 4×10⁴ cells/well. After overnight recovery, cells were treated with either a lysozyme solution (mock) or muropeptides (500 μg/ml) for 24 h. To prepare cell suspension, fibroblast cells were dissociated from culture surfaces by 0.25% Trypsin-EDTA (Gibco, 25200056) and complete growth media was used to inactivate trypsin. A Hemocytometer and Trypan Blue were used to count the live and dead cells.

FRDA fibroblasts were challenged with L-Buthionine-sulfoximine (BSO) (Sigma, B2515) according to an established protocol (Jauslin et al., 2003; Jauslin et al., 2002).

Transmission electron microscopy: Cells were grown on sapphire discs and prepared for electron microscopy using high pressure freezing and freeze substitution as described in (McDonald et al., 2010). Briefly, 3 mm sapphire discs (Technotrade International) were coated with gold and a large F was then scratched into the surface to help orient the cell side. The disks were coated with collagen, sterilized under UV light and cells were plated for culturing. Monolayers grown on sapphire discs were frozen using a Wohlwend Compact 02 high pressure freezer (Technotrade International). The frozen cells were then freeze substituted in 1% OsO₄ and 0.1% uranyl acetate in acetone at −80° C. for 3 days then gradually warmed to room temperature. The discs were flat embedded in a thin layer of Epon resin and polymerized at 60° C. Regions containing cells were identified in the light microscope, and a small square of resin containing the cells was excised and remounted onto a blank resin block. Thin (80 nm) sections were cut using a Leica Ultracut UCT and collected onto formvar-coated slot grids. Grids were post stained with 2% uranyl acetate and Reynolds lead citrate. The samples were imaged in a Tecnai T12 Spirit TEM operating at 100 kV (Thermo Fisher Scientific, Waltham, MA) using an AMT side-mount CCD camera.

Muropeptide fractionation: We followed an established method (Glauner et al., 1988) to separate muropeptides into monomeric, dimeric, and trimeric compounds. Briefly, unreduced muropeptides from lysozyme treatment were fractionated by gel filtration on Bio-Gel P6 (BIO-RAD, 150-4134) (2.5 cm/h; 100 mM LiCl in 20 mM sodium phosphate, PH 6.0). Fractions were collected as 1 ml/tube. Muropeptides were desalted by peptide desalting spin columns (Thermo Scientific, 89851) before use.

### TABLES

TABLE 1
Sub-library PG metabolism mutants

| Function | Activity | Proteins | Keio mutant | UPR^{mt} |
|---|---|---|---|---|
| Precursor synthesis | Transferase and | MurA | No | N/A |
| | deydrogenase | MurB | No | N/A |
| | Amino acid ligases | MUrC | No | N/A |
| | | MUrD | No | N/A |
| | | MUrE | No | N/A |
| | | MUrF | No | N/A |
| | | DdlA | Yes | No |
| | | DdlB | No | N/A |
| | Racemases | Alr | Yes | No |
| | | DadX | Yes | No |
| | | MurI | No | N/A |
| | GTases | MraY | No | N/A |
| | | MurG | No | N/A |
| Peptidoglycan synthesis | GTases and DD- | PBP1A | Yes | No |
| | Transpeptidases | | | |
| | (class A PBPs) | PBP1B | Yes | No |
| | DD-Transpeptidases | PBP2C | No | N/A |
| | (class B PBPs) | PBP3 | No | N/A |
| | GTase | MtgA | Yes | No |
| Regulation of peptidoglycan | Activators of peptidoglycan | LpoA | Yes | No |
| synthesis | synthase | LpoB | Yes | No |
| Formation of 3-3 crosslinks | LD-Transpeptidases | YnhG | Yes | No |
| | | YcbB | Yes | No |
| Cell envelope stability and creation | Structural protein | Lpp | Yes | No |
| of a firm connection between | LD-Transpeptidases | ErfK | Yes | Yes |
| peptidoglycan and the outer | | YbiS | Yes | No |
| membrane | | YcfS | Yes | No |
| peptidoglycan hydrolysis (autolysis) | DD-Carboxypeptidases | YfeW | Yes | No |
| | | DacA | Yes | No |
| | | DacC | Yes | No |
| | | DacD | Yes | No |
| | DD-Endopeptidases | DacB | Yes | Yes |
| | | PBP7 | No | N/A |
| | DD-carboxypeptidase and | AmpH | Yes | No |
| | DD-endopeptidase | | | |
| | DD-and LD-Endopeptidases | MepA | Yes | No |
| | LD-Carboxypeptidases | LdcA | Yes | No |
| | Endoamisase | MpaA | Yes | No |
| | L-Ala-D/L-Glu epimerase | YcjG | Yes | No |
| | D-Ala-D-Ala dipeptidase | DdpX | Yes | No |
| | β-N-acetylhexosaminidase | nagZ | Yes | No |
| | LTs | Slt70 | Yes | No |
| | | MltA | Yes | No |
| | | MltB | Yes | No |
| | | MltC | Yes | No |
| | | MitD | Yes | No |
| | | MitE | Yes | No |
| | | MltF | Yes | No |
| | Amidase | AmiA | Yes | No |
| | | AmiB | Yes | No |
| | | AmiC | Yes | No |
| | | AmpD | Yes | No |
| | | AmiD | Yes | Yes |
| | β-lactamase | ampC | Yes | No |
| Regulation of peptidoglycan | LytM-Domain Factors | EnvC | Yes | No |
| hydrolysis | | NlpD | Yes | No |
| | | YgeR | Yes | Yes |
| | | YebA | Yes | No |
| | Inhibitor of LTs | Ivy | Yes | No |
| Muropeptides recycle | Permease | AmpG | Yes | No |
| Cell elongation | Cytoskeletal structure | MreB | No | N/A |
| | MreB-associated proteins | MreC | No | N/A |
| | | MreD | No | N/A |
| | | Rodz | Yes | No |
| | | RodA | No | N/A |
| | | PBP2 | No | N/A |
| Cell division | Cytoskeletal structure | FtsZ | No | N/A |
| | Early association with the Z | FtsA | No | N/A |
| | ring | ZipA | No | N/A |
| | | ZapA | Yes | No |
| | | ZapB | Yes | No |
| | | ZapC | Yes | No |
| | | FtsE | No | N/A |
| | | FtsX | No | N/A |
| | | FtsK | No | N/A |
| | Late association with the Z | FtsQ | No | N/A |
| | ring | FtsL | No | N/A |
| | | FtsB | No | N/A |
| | | FtsW | No | N/A |
| | | FtsN | No | N/A |
| | | PBP3 | No | N/A |
| | | DamX | Yes | No |
| | | DedD | Yes | No |
| | | RlpA | Yes | No |
| | Outer-membrance | TolQ | Yes | No |
| | invagination | TolR | Yes | No |
| | | TolA | Yes | No |
| | | TolB | Yes | No |
| | | Pal | Yes | No |

TABLE 2
Potential protein interactors with PG

| | | Mol. | Sequence | | | |
|---|---|---|---|---|---|---|
| Gene | | weight | coverage | | | |
| names | Protein names | [kDa] | [%] | Score | Chrom | seq name |
| unc-54 | Myosin-4 | 224.75 | 48.1 | 323.31 | I | F11C3.3 |
| lev-11 | Tropomyosin isoforms | 33.003 | 79.9 | 323.31 | I | Y105E8B.1 |
| | a/b/d/f | | | | | |
| unc-15 | Paramyosin | 100.63 | 34.1 | 323.31 | I | F07A5.7 |
| tbb-2; | Tubulin beta-2 chain | 50.344 | 44.9 | 323.31 | III | C36E8.5 |
| let-75 | Myosin-1 | 223.32 | 26.6 | 323.31 | NO | NO |
| clik-1 | CaLponIn-like | 44.367 | 56.9 | 323.31 | V | T25F10.6 |
| | proteins | | | | | |
| myo-3 | Myosin-3 | 225.51 | 28.8 | 323.31 | V | K12F2.1 |
| ketn-1 | actin filament binding | 459.12 | 10.5 | 323.31 | V | F54E2.3 |
| | activit | | | | | |
| myo-5 | MYOsin heavy chain | 227 | 17.5 | 323.31 | V | F58G4.1 |
| | structural genes | | | | | |
| tcc-1 | Transmembrane and | 75.891 | 23.7 | 323.31 | V | Y59A8A.3 |
| | Coiled-Coil protein | | | | | |
| act-4; | Actin-4; Actin-2 | 40.426 | 67.4 | 323.31 | X | M03F4.2 |
| myo-2 | Myosin-2 | 223.05 | 30.7 | 323.31 | X | T18D3.4 |
| nmy-1 | Non-muscle MYosin | 229.37 | 16.7 | 323.31 | X | F52B10.1 |
| unc-87 | actin filament binding | 39.738 | 51.3 | 304.93 | I | F08B6.4 |
| | activity | | | | | |
| vit-6 | Vitellogenin-6 | 193.32 | 10.6 | 296.59 | IV | K07H8.6 |
| mup-2 | Troponin T | 47.041 | 39.3 | 295.05 | X | T22E5.5 |
| pqn-22 | Prion-like-(Q/N-rich)- | 119.63 | 27.1 | 282.96 | IV | C46G7.4 |
| | domain-bearing | | | | | |
| | protein | | | | | |
| car-1 | Cytokinesis, | 37.61 | 18.2 | 273.74 | I | Y18D10A.17 |
| | Apoptosis, RNA- | | | | | |
| | associated | | | | | |
| nmy-2 | Non-muscle MYosin | 231.24 | 11.2 | 266.98 | I | F20G4.3 |
| anc-1 | Nuclear anchorage | 840.47 | 10.5 | 264.6 | I | ZK973.6 |
| | protein 1 | | | | | |
| apt-1 | ATP synthase subunit | 57.787 | 24.9 | 245.63 | I | H28O16.1 |
| | alpha, mitochondrial | | | | | |
| vig-1 | SERPINE1 mRNA | 40.406 | 41.8 | 227.58 | II | F56D12.5 |
| | binding protein 1 | | | | | |
| rla-2 | Ribosomal protein, | 10.871 | 79.1 | 223.57 | IV | Y62E10A.1 |
| | Large subunit, Acidic | | | | | |
| | (P1 | | | | | |
| rps-0 | 40S ribosomal protein | 30.702 | 42.8 | 215.61 | III | B0393.1 |
| | SA | | | | | |
| lmn-1 | nuclear LaMiN | 64.083 | 26.7 | 213.92 | I | DY3.2 |
| mlc-3 | Myosin, essential light | 17.144 | 81 | 207.93 | III | F09F7.2 |
| | chain | | | | | |
| rps-15 | 40S ribosomal protein | 17.243 | 19.9 | 199.24 | I | F36A2.6 |
| | S15 | | | | | |
| rps-17 | 40S ribosomal protein | 14.938 | 56.9 | 184.28 | I | T08B2.10 |
| | S17 | | | | | |
| rme-1 | Receptor Mediated | 63.345 | 22.1 | 183.56 | V | W06H8.1 |
| | Endocytosis | | | | | |
| kars-1 | Lysine--tRNA ligase | 67.883 | 16.9 | 173.92 | II | T02G5.9 |
| larp-1 | La-related protein 1 | 128.27 | 20.5 | 170.98 | III | R144.7 |
| spc-1 | spc-1 | 281.75 | 5 | 170.48 | X | K10B3.10 |
| C18B2.5 | GTPase activity and | 74.809 | 17.7 | 170.21 | X | C18B2.5 |
| | protein-containing | | | | | |
| | complex scaffold | | | | | |
| | activity | | | | | |
| rpl-18 | 60S ribosomal protein | 20.996 | 34 | 167.64 | IV | Y45F10D.12 |
| | L18 | | | | | |
| F49E2.5 | No description | 113.44 | 10.7 | 167.12 | X | F49E2.5 |
| hsp-1 | Heat shock 70 kDa | 69.722 | 28.1 | 164.48 | IV | F26D10.3 |
| | protein A | | | | | |
| sqd-1 | ortholog of human | 33.467 | 34.1 | 164.23 | IV | Y73B6BL.6 |
| | HNRNPAB | | | | | |
| | (heterogeneous nuclear | | | | | |
| | ribonucleoprotein | | | | | |
| | A/B) | | | | | |
| vit-5 | Vitellogenin-5 | 186.44 | 7.2 | 164.08 | X | C04F6.1 |
| act-5 | ACTin | 41.872 | 47.2 | 162.81 | III | T25C8.2 |
| rpa-2 | 60S acidic ribosomal | 10.813 | 91.6 | 152.44 | I | M04F3.1 |
| | protein P2 | | | | | |
| rpa-0 | 60S acidic ribosomal | 33.774 | 48.4 | 149.99 | I | F25H2.10 |
| | protein P0 | | | | | |
| rps-8 | 40S ribosomal protein | 23.75 | 44.7 | 144 | IV | F42C5.8 |
| | S8 | | | | | |
| cey-2 | C. Elegans Y-box | 29.404 | 39.7 | 136.37 | I | F46F11.2 |
| rps-12 | 40S ribosomal protein | 15.069 | 44.3 | 135.77 | III | F54E7.2 |
| | S12 | | | | | |
| atp-4 | ATP synthase subunit | 13.957 | 42.6 | 132.81 | V | T05H4.12 |
| tnt-2 | TropoNin T | 48.684 | 24.5 | 132.25 | X | F53A9.10 |
| dlg-1 | discs large MAGUK | 106.99 | 4.4 | 131.09 | X | C25F6.2 |
| | scaffold protein 1 | | | | | |
| ucr-1 | Cytochrome b-c1 | 51.948 | 21.8 | 130.38 | III | F56D2.1 |
| | complex subunit 1, | | | | | |
| | mitochondrial | | | | | |
| frm-1 | FERM domain | 512.47 | 2.3 | 127.53 | I | ZK270.2 |
| | (protein4.1-ezrin- | | | | | |
| | radixin-moesin) family | | | | | |
| mig-6 | Papilin | 237.6 | 4.3 | 122.65 | V | C37C3.6 |
| F27D4.1 | Probable electron | 34.454 | 29.5 | 122.22 | I | F27D4.1 |
| | transfer flavoprotein | | | | | |
| | subunit alpha, | | | | | |
| | mitochondrial | | | | | |
| mdh-2 | Probable malate | 35.119 | 33.4 | 122.2 | III | F20H11.3 |
| | dehydrogenase, | | | | | |
| | mitochondrial | | | | | |
| pqn-70 | (Prion-like-(Q/N-rich)- | 26.165 | 6.6 | 120.89 | V | T19B10.4 |
| | domain-bearing | | | | | |
| | protein | | | | | |
| ifb-1 | Intermediate filament | 67.148 | 8.8 | 119.78 | II | F10C1.2 |
| | protein ifb-1 | | | | | |
| vit-2 | Vitellogenin-2 | 180.92 | 13 | 119.53 | X | C42D8.2 |
| daf-21 | Heat shock protein 90 | 80.282 | 19.5 | 119.33 | V | C47E8.5 |
| tni-3 | Troponin I 3 | 29.838 | 23.1 | 117.62 | V | T20B3.2 |
| rps-19 | 40S ribosomal protein | 16.322 | 61.6 | 114.38 | I | T05F1.3 |
| | S19 | | | | | |
| cey-1 | C. Elegans Y-box | 21.349 | 27.4 | 114.32 | II | F33A8.3 |
| rps-5 | 40S ribosomal protein | 23.154 | 36.7 | 113.97 | IV | T05E11.1 |
| | S5 | | | | | |
| rps-25 | 40S ribosomal protein | 12.912 | 13.7 | 113.85 | IV | K02B2.5 |
| | S25 | | | | | |
| F21C10.7 | ortholog of human | 295.08 | 2.1 | 110.92 | V | F21C10.7 |
| | CCDC141 (coiled-coil | | | | | |
| | domain containing | | | | | |
| | 141) | | | | | |
| rpl-8 | 60S ribosomal protein | 28.203 | 28.5 | 107.3 | IV | Y24D9A.4 |
| | L8 | | | | | |
| cpl-1 | CathePsin L family | 21.838 | 29.3 | 104.96 | V | T03E6.7 |
| glh-1 | ATP-dependent RNA | 79.779 | 13.4 | 103.66 | I | T21G5.3 |
| | helicase glh-1 | | | | | |
| rps-6 | 40S ribosomal protein | 28.135 | 25.2 | 102.53 | I | Y71A12B.1 |
| | S6 | | | | | |
| tkt-1 | TransKeTolase | 66.01 | 17.6 | 101.32 | IV | F01G10.1 |
| | homolog | | | | | |
| mlc-2 | Myosin regulatory | 18.603 | 68.8 | 101.22 | X | C36E6.5 |
| | light chain 2 | | | | | |
| rps-20 | Ribosomal Protein, | 13.234 | 43.6 | 100.93 | I | Y105E8A.16 |
| | Small subunit | | | | | |
| unc-27 | Troponin I 2 | 27.56 | 26 | 98.773 | X | ZK721.2 |
| rps-21 | 40S ribosomal protein | 9.6748 | 59.1 | 98.538 | III | F37C12.11 |
| | S21 | | | | | |
| hmg-12 | DNA-(apurinic or | 33.004 | 37.5 | 97.649 | II | Y17G7A.1 |
| | apyrimidinic site) | | | | | |
| | endonuclease activity | | | | | |
| rpl-21 | 60S ribosomal protein | 18.31 | 23.6 | 95.312 | III | C14B9.7 |
| | L21 | | | | | |
| rpl-11.2 | Ribosomal Protein, | 22.776 | 18.9 | 92.863 | X | F07D10.1 |
| | Large subunit | | | | | |
| rpl-7A | 60S ribosomal protein | 30.174 | 20.4 | 92.529 | IV | Y24D9A.4 |
| | L7a | | | | | |
| immt-1 | Inner Membrane of | 75.996 | 5.3 | 90.359 | X | T14G11.3 |
| | MiTochondria protein | | | | | |
| | homolog | | | | | |
| Y39B6A.33 | Uncharacterized | 49.746 | 17.4 | 89.116 | V | Y39B6A.33 |
| | protein Y39B6A.33 | | | | | |
| glrx-5 | Glutaredoxin | 15.768 | 12 | 86.441 | III | Y49E10.2 |
| clik-2 | Uncharacterized | 44.815 | 19.9 | 85.949 | X | C53C9.2 |
| | protein C53C9.2 | | | | | |
| T23E7.2 | No description | 98.283 | 6.3 | 84.636 | X | T23E7.2 |
| rpl-32 | Ribosomal Protein, | 9.0994 | 22.2 | 84.335 | | T24B8.1 |
| | Large subunit | | | | | |
| tni-4 | Troponin I 4 | 23.059 | 16.5 | 83.575 | IV | W03F8.1 |
| chch-3 | Coiled coil Helix | 19.193 | 25.4 | 83.379 | II | M176.3 |
| | Coiled coiled Helix | | | | | |
| | domain | | | | | |
| eef-2 | Elongation factor 2 | 94.795 | 5.2 | 83.194 | I | F25H5.4 |
| rpl-27 | 60S ribosomal protein | 15.728 | 33.1 | 82.785 | I | C53H9.1 |
| | L27 | | | | | |
| tln-1 | TaLIN | 70.526 | 12.8 | 81.695 | I | Y71G12B.11 |
| vab-10 | actin filament binding | 374.91 | 5.4 | 80.995 | I | ZK1151.1 |
| | activity and structural | | | | | |
| | molecule activity | | | | | |
| Y75B8A.7 | ortholog of human | 73.896 | 6.5 | 79.397 | III | Y75B8A.7 |
| | MPHOSPH10 (M- | | | | | |
| | phase phosphoprotein | | | | | |
| | 10 | | | | | |
| rpl-19 | 60S ribosomal protein | 23.651 | 24.2 | 78.933 | I | C09D4.5 |
| | L19 | | | | | |
| rps-22 | Ribosomal Protein, | 14.733 | 12.3 | 78.419 | III | F53A3.3 |
| | Small subunit | | | | | |
| hrpk-1 | eterogeneous nuclear | 40.525 | 7.6 | 77.953 | I | F26B1.2 |
| | RibonucleoProtein | | | | | |
| | (HnRNP) K homolog | | | | | |
| tsn-1 | Tudor Staphylococcal | 100.77 | 4.4 | 77.109 | II | F10G7.2 |
| | Nuclease homolog | | | | | |
| rpl-5 | 60S ribosomal protein | 33.386 | 17.7 | 77.034 | II | F54C9.5 |
| | L5 | | | | | |
| rpl-35 | 60S ribosomal protein | 14.195 | 8.1 | 75.952 | III | ZK652.4 |
| | L35 | | | | | |
| fars-1 | Phenylalanyl Amino- | 56.182 | 10.3 | 74.298 | I | T08B2.9 |
| | acyl tRNA Synthetase | | | | | |
| pars-1 | Prolyl Amino-acyl | 51.586 | 7.7 | 74.236 | III | T20H4.3 |
| | tRNA Synthetase | | | | | |
| nrfl-1 | mammalian Na/H | 44.85 | 19.8 | 73.888 | IV | C01F6.6 |
| | Exchange Regulatory | | | | | |
| | Factor | | | | | |
| vha-16 | Vacuolar H ATPase | 39.937 | 8 | 73.606 | I | C30F8.2 |
| mlc-4 | Probable myosin | 19.94 | 34.9 | 73.387 | III | C56G7.1 |
| | regulatory light chain | | | | | |
| aars-2 | Alanine--tRNA ligase, | 106.78 | 4.5 | 72.52 | I | F28H1.3 |
| | cytoplasmic | | | | | |
| tba-1; | TuBulin, Alpha | 50.025 | 16.7 | 72.515 | I | F26E4.8 |
| rpl-9 | 60S ribosomal protein | 21.508 | 33.3 | 71.352 | III | R13A5.8 |
| | L9 | | | | | |
| his-11 | Histone H2B | 13.5 | 27 | 71.035 | II | ZK131.5 |
| | 1; Histone H2B | | | | | |
| | 2; Probable histone | | | | | |
| | H2B 4; Probable | | | | | |
| | histone H2B 3 | | | | | |
| dnc-2 | Probable dynactin | 37.154 | 26.9 | 70.976 | III | C28H8.12 |
| | subunit 2 | | | | | |
| F46H5.7 | No description | 64.271 | 9.8 | 69.684 | X | F46H5.7 |
| rpb-9 | DNA-directed RNA | 19.335 | 21 | 68.717 | V | Y97E10AR.5 |
| | polymerase subunit | | | | | |
| ctsa-2 | Carboxypeptidase | 256.43 | 3 | 68.38 | X | K10C2.1 |
| eif-2 | Eukaryotic Initiation | 51.234 | 7.7 | 67.503 | II | E04D5.1 |
| | Factor | | | | | |
| gei-15 | GEX Interacting | 44.577 | 31.8 | 66.185 | X | M03A8.4 |
| | protein | | | | | |
| hsp-25 | Heat Shock Protein | 12.417 | 25.9 | 65.453 | X | C09B8.6 |
| ubl-1 | Ubiquitin-like protein | 18.098 | 25.2 | 65.127 | III | H06I04.4 |
| | 1-40S ribosomal | | | | | |
| | protein | | | | | |
| | S27a; Ubiquitin-like | | | | | |
| | protein 1; 40S | | | | | |
| | ribosomal protein | | | | | |
| | S27a | | | | | |
| lfi-1 | in-5 (Five) Interacting | 242.46 | 8.8 | 65.062 | X | ZC8.4 |
| | protein | | | | | |
| let-805 | involved in | 497.45 | 2.1 | 63.992 | III | H19M22.2 |
| | hemidesmosome | | | | | |
| | assembly | | | | | |
| rpl-36 | 60S ribosomal protein | 11.884 | 48.1 | 62.813 | III | F37C12.4 |
| | L36 | | | | | |
| F23C8.5 | electron transfer | 27.64 | 50.2 | 62.545 | I | F23C8.5 |
| | flavoprotein subunit | | | | | |
| | beta, mitochondria | | | | | |
| vgln-1 | ViGiLN homolog | 134.34 | 5.4 | 62.445 | II | C08H9.2 |
| C18H9.3/ | GYF domain- | 120.8 | 8.6 | 62.22 | II | C18H9.3 |
| | containing protein | | | | | |
| | C18H9.3 | | | | | |
| rps-3 | 40S ribosomal protein | 27.313 | 29.6 | 61.405 | III | C23G10.3 |
| | S3 | | | | | |
| rpac-19 | Probable DNA- | 16.594 | 50 | 59.673 | III | F58A4.9 |
| | directed RNA | | | | | |
| | polymerases I and III | | | | | |
| | subunit RPAC2 | | | | | |
| rps-13 | 40S ribosomal protein | 17.316 | 19.9 | 58.177 | III | C16A3.9 |
| | S13 | | | | | |
| hpo-34 | Hypersensitive to | 85.647 | 34.3 | 58.093 | X | F29G6.3 |
| | POre-forming toxin | | | | | |
| alp-1 | ortholog of human | 45.883 | 12.5 | 57.482 | IV | T11B7.4 |
| | LDB3 (LIM domain | | | | | |
| | binding 3) | | | | | |
| rps-10 | Ribosomal Protein, | 16.87 | 18.8 | 57.208 | I | D1007.6 |
| | Small subunit | | | | | |
| vrs-2 | Valine--tRNA ligase | 118.92 | 6.6 | 56.368 | I | Y87G2A.5 |
| ant-1.1 | Adenine Nucleotide | 33.044 | 15.7 | 56.067 | III | T27E9.1 |
| | Translocator, | | | | | |
| | mitochondria | | | | | |
| dlat-1 | Dihydrolipoyllysine- | 53.466 | 6.7 | 55.273 | V | F23B12.5 |
| | residue | | | | | |
| | acetyltransferase | | | | | |
| | component of pyruvate | | | | | |
| | dehydrogenase | | | | | |
| | complex, | | | | | |
| | mitochondrial | | | | | |
| eef-la.2; | Elongation factor 1- | 47.145 | 26.6 | 54.622 | X | R03G5.1 |
| | alpha | | | | | |
| rpl-25.2 | 60S ribosomal protein | 16.283 | 40.4 | 54.084 | I | F52B5.6 |
| | L23a 2 | | | | | |
| rla-1 | 60S acidic ribosomal | 11.284 | 93.7 | 52.044 | I | Y37E3.7 |
| | protein P1 | | | | | |
| rpn-3 | 26S proteasome non- | 57.506 | 8.5 | 52.032 | III | C30C11.2 |
| | ATPase regulatory | | | | | |
| | subunit 3 | | | | | |
| T09B4.5 | No description | 36.425 | 4.9 | 50.966 | I | T09B4.5 |
| mlc-5 | Myosin Light Chain | 15.97 | 41.5 | 50.67 | III | T12D8.6 |
| ears-1 | glutamyl(E) Amino- | 125.2 | 5.4 | 50.529 | I | ZC434.5 |
| | acyl tRNA Synthetase | | | | | |
| rps-2 | 40S ribosomal protein | 28.96 | 12.1 | 50.156 | IV | C49H3.11 |
| | S2 | | | | | |
| atp-2 | ATP synthase subunit | 57.526 | 21.2 | 49.198 | III | C34E10.6 |
| | beta, mitochondrial | | | | | |
| asb-1 | ATP Synthase B | 34.368 | 3.3 | 48.531 | III | F35G12.10 |
| | homolog | | | | | |
| sdha-1; | Succinate | 70.397 | 5.6 | 47.726 | X | C03G5.1 |
| | dehydrogenase | | | | | |
| | [ubiquinone] | | | | | |
| | flavoprotein subunit, | | | | | |
| | mitochondrial | | | | | |
| rpl-4 | 60S ribosomal protein | 38.659 | 18.8 | 46.483 | I | B0041.4 |
| | L4 | | | | | |
| egl-45 | Eukaryotic translation | 124.41 | 2.5 | 46.36 | III | C27D11.1 |
| | initiation factor 3 | | | | | |
| | subunit A | | | | | |
| pqn-52 | Prion-like-(Q/N-rich)- | 62.591 | 3.9 | 45.569 | I | M01E11.4 |
| | domain-bearing | | | | | |
| | protein | | | | | |
| acdh-12 | Acyl CoA | 66.171 | 13.5 | 44.884 | II | E04F6.5 |
| | DeHydrogenase | | | | | |
| mlc-1 | Myosin regulatory | 18.617 | 68.8 | 44.804 | X | C36E6.3 |
| | light chain 1 | | | | | |
| pat-12 | Paralysed Arrest at | 76.708 | 9.8 | 44.658 | III | T17H7.4 |
| | Two-fold | | | | | |
| dtmk-1 | Probable thymidylate | 24.342 | 39.9 | 44.611 | II | R53.2 |
| | kinase | | | | | |
| rps-18 | Ribosomal Protein, | 17.77 | 33.8 | 44.499 | IV | Y57G11C.16 |
| | Small subunit | | | | | |
| nuo-5 | ortholog of human | 79.356 | 6.7 | 44.187 | V | Y45G12B.1 |
| | NDUFS1 | | | | | |
| | (NADH:ubiquinone | | | | | |
| | oxidoreductase core | | | | | |
| | subunit S1) | | | | | |
| ifet-1 | eIF4E Transporter | 83.95 | 19.6 | 43.901 | III | F56F3.1 |
| lin-53 | Probable histone- | 47.166 | 18.9 | 43.841 | I | K07A1.12 |
| | binding protein lin-53 | | | | | |
| icd-2 | Nascent polypeptide- | 22.077 | 34.5 | 42.797 | I | Y65B4BR.5 |
| | associated complex | | | | | |
| | subunit alpha | | | | | |
| mec-7 | Tubulin beta-1 chain | 49.26 | 14.5 | 42.56 | X | ZK154.3 |
| F21D5.1 | ortholog of human | 61.077 | 2.9 | 42.524 | IV | F21D5.1 |
| | PGM3 | | | | | |
| | (phosphoglucomutase | | | | | |
| | 3) | | | | | |
| pat-10 | troponin I binding | 18.516 | 44.1 | 42.027 | I | F54C1.7 |
| | activity | | | | | |
| rps-1 | 40S ribosomal protein | 28.96 | 24.9 | 42.009 | III | F56F3.5 |
| | S3a | | | | | |
| nkb-3; | Probable | 34.234 | 5.6 | 41.805 | X | F55F3.3 |
| | sodium/potassium- | | | | | |
| | transporting ATPase | | | | | |
| | subunit beta- | | | | | |
| | 3; Sodium/potassium- | | | | | |
| | transporting ATPase | | | | | |
| | subunit beta-1 | | | | | |
| gfi-1 | GEI-4 (Four) | 221.16 | 3.5 | 41.464 | V | F57F4.3 |
| | Interacting protein | | | | | |
| his-24 | Histone H1.1 | 21.366 | 6.2 | 40.739 | X | M163.3 |
| tni-1 | Troponin I 1 | 28.892 | 20.4 | 40.685 | X | F42E11.4 |
| eif-2alpha | Eukaryotic Initiation | 38.898 | 3.8 | 40.556 | I | Y37E3.10 |
| | Factor | | | | | |
| baf-1 | Barrier-to- | 9.9542 | 43.8 | 39.429 | III | B0464.7 |
| | autointegration factor | | | | | |
| | 1 | | | | | |
| npp-13 | Nuclear Pore complex | 88.279 | 6.5 | 39.379 | I | Y37E3.15 |
| | Protein | | | | | |
| lin-22 | ortholog of human HES1 | 24 | 8 | 39.092 | IV | Y54G2A.1 |
| | (hes family bHLH | | | | | |
| | transcription factor 1) | | | | | |
| ifo-1 | Intermediate Filament | 148.45 | 6.3 | 38.755 | IV | F42C5.10 |
| | Organize | | | | | |
| atp-5 | ATP synthase subunit | 21.797 | 47.1 | 37.238 | V | C06H2.1 |
| unc-70 | UNCoordinated | 262.22 | 2.3 | 37.191 | V | K11C4.3 |
| hsp-3 | Heat shock 70 kDa | 73.022 | 15 | 36.666 | X | C15H9.6 |
| | protein C | | | | | |
| pab-1 | PolyA Binding protein | 65.092 | 3.9 | 36.514 | I | Y106G6H.2 |
| F27D4.4 | Zinc finger CCCH | 43.301 | 11.8 | 36.194 | I | F27D4.4 |
| | domain-containing | | | | | |
| | protein 15 homolog | | | | | |
| tim-8 | Mitochondrial import | 9.5778 | 30.1 | 35.903 | III | Y39A3CR.4 |
| | inner membrane | | | | | |
| | translocase subunit | | | | | |
| | tim-8 | | | | | |

### REFERENCES

The following references are hereby incorporated by references related specifically to the production and use of muropeptides.
1. Barratt, M. J., Lebrilla, C., Shapiro, H. Y. & Gordon, J. I. The Gut Microbiota, Food Science, and Human Nutrition: A Timely Marriage. Cell Host Microbe 22, 134-141, doi:10.1016/j.chom.2017.07.006 (2017).
2. Brown, J. M. & Hazen, S. L. Microbial modulation of cardiovascular disease. Nat Rev Microbiol 16, 171-181, doi:10.1038/nrmicro.2017.149 (2018).
3. Kundu, P., Blacher, E., Elinav, E. & Pettersson, S. Our Gut Microbiome: The Evolving Inner Self. Cell 171, 1481-1493, doi:10.1016/j.cell.2017.11.024 (2017).
4. Postler, T. S. & Ghosh, S. Understanding the Holobiont: How Microbial Metabolites Affect Human Health and Shape the Immune System. Cell Metab 26, 110-130, doi:10.1016/j.cmet.2017.05.008 (2017).
5. Fan, Y. & Pedersen, O. Gut microbiota in human metabolic health and disease. Nature reviews. Microbiology, doi:10.1038/s41579-020-0433-9 (2020).
6. Cani, P. D. Human gut microbiome: hopes, threats and promises. Gut 67, 1716-1725, doi:10.1136/gutjnl-2018-316723 (2018).
7. Fischbach, M. A. Microbiome: Focus on Causation and Mechanism. Cell 174, 785-790, doi:10.1016/j.cell.2018.07.038 (2018).
8. Donia, M. S. & Fischbach, M. A. HUMAN MICROBIOTA. Small molecules from the human microbiota. Science 349, 1254766, doi:10.1126/science. 1254766 (2015).
9. Garcia-Gonzalez, A. P. et al. Bacterial Metabolism Affects the C. elegans Response to Cancer Chemotherapeutics. Cell 169, 431-441 e438, doi:10.1016/j.cell.2017.03.046 (2017).
10. Heintz, C. & Mair, W. You are what you host: microbiome modulation of the aging process. Cell 156, 408-411, doi:10.1016/j.cell.2014.01.025 (2014).
11. Schleifer, K. H. & Kandler, O. Peptidoglycan types of bacterial cell walls and their taxonomic implications. Bacteriological reviews 36, 407-477 (1972).
12. Bourhis, L. L. & Werts, C. Role of Nods in bacterial infection. Microbes and infection 9, 629-636, doi:10.1016/j.micinf.2007.01.014 (2007).
13. Humann, J. & Lenz, L. L. Bacterial peptidoglycan degrading enzymes and their impact on host muropeptide detection. Journal of innate immunity 1, 88-97, doi:10.1159/000181181 (2009).
14. Irazoki, O., Hernandez, S. B. & Cava, F. Peptidoglycan Muropeptides: Release, Perception, and Functions as Signaling microbiology Molecules. Frontiers in 10, 500, doi:10.3389/fmicb.2019.00500 (2019).
15. McDonald, C., Inohara, N. & Nunez, G. Peptidoglycan signaling in innate immunity and inflammatory disease. The Journal of biological chemistry 280, 20177-20180, doi:10.1074/jbc.R500001200 (2005).
16. Gottar, M. et al. The Drosophila immune response against Gram-negative bacteria is mediated by a peptidoglycan recognition protein. Nature 416, 640-644, doi:10.1038/nature734 (2002).
17. Girardin, S. E. et al. Nod2 is a general sensor of peptidoglycan through muramyl dipeptide (MDP) detection. The Journal of biological chemistry 278, 8869-8872, doi:10.1074/jbc.C200651200 (2003).
18. Palaniyar, N., Nadesalingam, J. & Reid, K. B. Pulmonary innate immune proteins and receptors that interact with gram-positive bacterial ligands. Immunobiology 205, 575-594, doi:10.1078/0171-2985-00156 (2002).
19. Arentsen, T., Khalid, R., Qian, Y. & Diaz Heijtz, R. Sex-dependent alterations in motor and anxiety-like behavior of aged bacterial peptidoglycan sensing molecule 2 knockout mice. Brain, behavior, and immunity 67, 345-354, doi:10.1016/j.bbi.2017.09.014 (2018).
20. Baba, T. et al. Construction of Escherichia coli K-12 in-frame, single-gene knockout mutants: the Keio collection. Molecular systems biology 2, 2006 0008, doi:10.1038/msb4100050 (2006).
21. Qi, B. & Han, M. Microbial Siderophore Enterobactin Promotes Mitochondrial Iron Uptake and Development of the Host via Interaction with ATP Synthase. Cell 175, 571-582 e511, doi:10.1016/j.cell.2018.07.032 (2018).
22. Govindan, J. A., Jayamani, E., Zhang, X., Mylonakis, E. & Ruvkun, G. Dialogue between E. coli free radical pathways and the mitochondria of C. elegans. Proceedings of the National Academy of Sciences of the United States of America 112, 12456-12461, doi:10.1073/pnas. 1517448112 (2015).
23. Zhang, J. et al. A Delicate Balance between Bacterial Iron and Reactive Oxygen Species Supports Optimal C. elegans Development. Cell host & microbe 26, 400-411 e403, doi:10.1016/j.chom.2019.07.010 (2019).
24. Qi, B., Kniazeva, M. & Han, M. A vitamin-B2-sensing mechanism that regulates gut protease activity to impact animal's food behavior and growth. eLife 6, doi:10.7554/eLife.26243 (2017).
25. Kniazeva, M., Zhu, H., Sewell, A. K. & Han, M. A Lipid-TORC1 Pathway Promotes Neuronal Development and Foraging Behavior under Both Fed and Fasted Conditions in C. elegans. Developmental cell 33, 260-271, doi:10.1016/j.devcel.2015.02.015 (2015).
26. Melo, J. A. & Ruvkun, G. Inactivation of conserved C. elegans genes engages pathogen- and xenobiotic-associated defenses. Cell 149, 452-466, doi:10.1016/j.cell.2012.02.050 (2012).
27. Artyukhin, A. B., Yim, J. J., Cheong Cheong, M. & Avery, L. Starvation-induced collective behavior in C. elegans. Scientific reports 5, 10647, doi:10.1038/srep10647 (2015).
28. Liu, Y., Samuel, B. S., Breen, P. C. & Ruvkun, G. Caenorhabditis elegans pathways that surveil and defend mitochondria. Nature 508, 406-410, doi:10.1038/nature13204 (2014).
29. Shtonda, B. B. & Avery, L. Dietary choice behavior in Caenorhabditis elegans. The Journal of experimental biology 209, 89-102, doi:10.1242/jeb.01955 (2006).
30. Brandt, J. P. & Ringstad, N. Toll-like Receptor Signaling Promotes Development and Function of Sensory Neurons Required for a C. elegans Pathogen-Avoidance Behavior. Current biology: CB 25, 2228-2237, doi:10.1016/j.cub.2015.07.037 (2015).
31. Meisel, J. D., Panda, O., Mahanti, P., Schroeder, F. C. & Kim, D. H. Chemosensation of bacterial secondary metabolites modulates neuroendocrine signaling and behavior of C. elegans. Cell 159, 267-280, doi:10.1016/j.cell.2014.09.011 (2014).
32. Kuhner, D., Stahl, M., Demircioglu, D. D. & Bertsche, U. From cells to muropeptide structures in 24 h: peptidoglycan mapping by UPLC-MS. Scientific reports 4, 7494, doi:10.1038/srep07494 (2014).
33. Labbadia, J. & Morimoto, R. I. The biology of proteostasis in aging and disease. Annual review of biochemistry 84, 435-464, doi:10.1146/annurev-biochem-060614-033955 (2015).
34. Shpilka, T. & Haynes, C. M. The mitochondrial UPR: mechanisms, physiological functions and implications in ageing. Nature reviews. Molecular cell biology 19, 109-120, doi:10.1038/nrm.2017.110 (2018).
35. Frakes, A. E. & Dillin, A. The UPR (ER): Sensor and Coordinator of Organismal Homeostasis. Molecular cell 66, 761-771, doi:10.1016/j.molcel.2017.05.031 (2017).
36. Kitamura, M. The unfolded protein response triggered by environmental factors. Seminars in immunopathology 35, 259-275, doi:10.1007/s00281-013-0371-y (2013).
37. Grootjans, J., Kaser, A., Kaufman, R. J. & Blumberg, R. S. The unfolded protein response in immunity and inflammation. Nature reviews. Immunology 16, 469-484, doi:10.1038/nri.2016.62 (2016).
38. Mitra, S. & Ryoo, H. D. The unfolded protein response in metazoan development. Journal of cell science 132, doi:10.1242/jcs.217216 (2019).
39. Taylor, R. C., Berendzen, K. M. & Dillin, A. Systemic stress signalling: understanding the cell non-autonomous control of proteostasis. Nature reviews. Molecular cell biology 15, 211-217, doi:10.1038/nrm3752 (2014).
40. Anderson, N. S. & Haynes, C. M. Folding the Mitochondrial UPR into the Integrated Stress Response. Trends in cell biology 30, 428-439, doi:10.1016/j.tcb.2020.03.001 (2020).
41. Girardin, S. E. et al. Nod1 detects a unique muropeptide from gram-negative bacterial peptidoglycan. Science 300, 1584-1587, doi:10.1126/science.1084677 (2003).
42. Royet, J., Gupta, D. & Dziarski, R. Peptidoglycan recognition proteins: modulators of the microbiome and inflammation. Nature reviews. Immunology 11, 837-851, doi:10.1038/nri3089 (2011).
43. Dingley, S. et al. Mitochondrial respiratory chain dysfunction variably increases oxidant stress in Caenorhabditis elegans. Mitochondrion 10, 125-136, doi:10.1016/j.mito.2009.11.003 (2010).
44. Lee, S. J., Hwang, A. B. & Kenyon, C. Inhibition of respiration extends C. elegans life span via reactive oxygen species that increase HIF-1 activity. Current biology: CB 20, 2131-2136, doi:10.1016/j.cub.2010.10.057 (2010).
45. Libina, N., Berman, J. R. & Kenyon, C. Tissue-specific activities of C. elegans DAF-16 in the regulation of lifespan. Cell 115, 489-502, doi:10.1016/s0092-8674 (03) 00889-4 (2003).
46. Kraus, E. & Femfert, U. Proteinase K from the mold Tritirachium album Limber. Specificity and mode of action. Hoppe-Seyler's Zeitschrift fur physiologische Chemie 357, 937-947, doi:10.1515/bchm2.1976.357.2.937 (1976).
47. Mann, B., Loh, L. N., Gao, G. & Tuomanen, E. Preparation of Purified Gram-positive Bacterial Cell Wall and Detection in Placenta and Fetal Tissues. Bio-protocol 6, doi:10.21769/BioProtoc.2037 (2016).
48. Packiam, M., Weinrick, B., Jacobs, W. R., Jr. & Maurelli, A. T. Structural characterization of muropeptides from Chlamydia trachomatis peptidoglycan by mass spectrometry resolves “chlamydial anomaly”. Proceedings of the National Academy of Sciences of the United States of America 112, 11660-11665, doi:10.1073/pnas. 1514026112 (2015).
49. Baik, J. E. et al. Differential profiles of gastrointestinal proteins interacting with peptidoglycans from Lactobacillus plantarum and Staphylococcus aureus. Molecular immunology 65, 77-85, doi:10.1016/j.molimm.2015.01.007 (2015).
50. Alavian, K. N. et al. Bcl-xL regulates metabolic efficiency of neurons through interaction with the mitochondrial FIFO ATP synthase. Nature cell biology 13, 1224-1233, doi:10.1038/ncb2330 (2011).
51. Chin, R. M. et al. The metabolite alpha-ketoglutarate extends lifespan by inhibiting ATP synthase and TOR. Nature 510, 397-401, doi:10.1038/nature13264 (2014).
52. Esparza-Molto, P. B. & Cuezva, J. M. The Role of Mitochondrial H(+)-ATP Synthase in Cancer. Frontiers in oncology 8, 53, doi:10.3389/fonc.2018.00053 (2018).
53. Goldberg, J. et al. The mitochondrial ATP synthase is a shared drug target for aging and dementia. Aging cell 17, doi:10.1111/acel.12715 (2018).
54. Bason, J. V., Montgomery, M. G., Leslie, A. G. & Walker, J. E. Pathway of binding of the intrinsically disordered mitochondrial inhibitor protein to F1-ATPase. Proceedings of the National Academy of Sciences of the United States of America 111, 11305-11310, doi:10.1073/pnas. 1411560111 (2014).
55. Pullman, M. E. & Monroy, G. C. A Naturally Occurring Inhibitor of Mitochondrial Adenosine Triphosphatase. The Journal of biological chemistry 238, 3762-3769 (1963).
56. Campanella, M. et al. Regulation of mitochondrial structure and function by the FlFo-ATPase inhibitor protein, IF1. Cell metabolism 8, 13-25, doi:10.1016/j.cmet.2008.06.001 (2008).
57. Garcia-Aguilar, A. & Cuezva, J. M. A Review of the Inhibition of the Mitochondrial ATP Synthase by IF1 in vivo: Reprogramming Energy Metabolism and Inducing Mitohormesis. Frontiers in physiology 9, 1322, doi:10.3389/fphys.2018.01322 (2018).
58. Garcia-Bermudez, J. et al. PKA Phosphorylates the ATPase Inhibitory Factor 1 and Inactivates Its Capacity to Bind and Inhibit the Mitochondrial H (+)-ATP Synthase. Cell reports 12, 2143-2155, doi:10.1016/j.celrep.2015.08.052 (2015).
59. Ichikawa, N., Ando, C. & Fumino, M. Caenorhabditis elegans MAI-1 protein, which is similar to mitochondrial ATPase inhibitor (IF1), can inhibit yeast F0F1-ATPase but cannot be transported to yeast mitochondria. Journal of bioenergetics and biomembranes 38, 93-99, doi:10.1007/s10863-006-9009-2 (2006).
60. Fernandez-Cardenas, L. P. et al. Caenorhabditis elegans ATPase inhibitor factor 1 (IF1) MAI-2 preserves the mitochondrial membrane potential (Deltapsim) and is important to induce germ cell apoptosis. PloS one 12, e0181984, doi:10.1371/journal.pone.0181984 (2017).
61. Sambuy, Y. et al. The Caco-2 cell line as a model of the intestinal barrier: influence of cell and culture-related factors on Caco-2 cell functional characteristics. Cell biology and toxicology 21, 1-26, doi:10.1007/s10565-005-0085-6 (2005).
62. Brand, M. D. & Nicholls, D. G. Assessing mitochondrial dysfunction in cells. The Biochemical journal 435, 297-312, doi:10.1042/BJ20110162 (2011).
63. Pastores, G. M. et al. Leigh syndrome and hypertrophic cardiomyopathy in an infant with a mitochondrial DNA point mutation (T8993G). American journal of medical genetics 50, 265-271, doi:10.1002/ajmg.1320500310 (1994).
64. Houstek, J. et al. Altered properties of mitochondrial ATP-synthase in patients with a T-->G mutation in the ATPase 6 (subunit a) gene at position 8993 of mtDNA. Biochimica et biophysica acta 1271, 349-357, doi:10.1016/0925-4439 (95) 00063-a (1995).
65. Cortes-Hernandez, P., Vazquez-Memije, M. E. & Garcia, J. J. ATP6 homoplasmic mutations inhibit and destabilize the human FIFO-ATP synthase without preventing enzyme assembly and oligomerization. The Journal of biological chemistry 282, 1051-1058, doi:10.1074/jbc.M606828200 (2007).
66. Nijtmans, L. G., Henderson, N. S., Attardi, G. & Holt, I. J. Impaired ATP synthase assembly associated with a mutation in the human ATP synthase subunit 6 gene. The Journal of biological chemistry 276, 6755-6762, doi:10.1074/jbc.M008114200 (2001).
67. Vazquez-Memije, M. E. et al. Comparative biochemical studies of ATPases in cells from patients with the T8993G or T8993C mitochondrial DNA mutations. Journal of inherited metabolic disease 21, 829-836, doi:10.1023/a: 1005418718299 (1998).
68. Tatuch, Y. et al. Heteroplasmic mtDNA mutation (T---G) at 8993 can cause Leigh disease when the percentage of abnormal mtDNA is high. American journal of human genetics 50, 852-858 (1992).
69. Trounce, I., Neill, S. & Wallace, D. C. Cytoplasmic transfer of the mtDNA nt 8993 T-->G (ATP6) point mutation associated with Leigh syndrome into mtDNA-less cells demonstrates cosegregation with a decrease in state III respiration and ADP/O ratio. Proceedings of the National Academy of Sciences of the United States of America 91, 8334-8338, doi:10.1073/pnas.91.18.8334 (1994).
70. Garcia, J. J., Ogilvie, I., Robinson, B. H. & Capaldi, R. A. Structure, functioning, and assembly of the ATP synthase in cells from patients with the T8993G mitochondrial DNA mutation. Comparison with the enzyme in Rho (0) cells completely lacking mtdna. The Journal of biological chemistry 275, 11075-11081, doi:10.1074/jbc.275.15.11075 (2000).
71. Carrozzo, R. et al. Maternally-inherited Leigh syndrome-related mutations bolster mitochondrial-mediated apoptosis. Journal of neurochemistry 90, 490-501, doi:10.1111/j.1471-4159.2004.02505.x (2004).
72. Henriques, M. et al. Mitochondrial DNA 8993T>G mutation in a child with ornithine transcarbamylase deficiency and leigh syndrome: an unexpected association. Journal of child neurology 27, 1059-1061, doi:10.1177/0883073811431015 (2012).
73. Baracca, A. et al. Biochemical phenotypes associated with the mitochondrial ATP6 gene mutations at nt8993. Biochimica et biophysica acta 1767, 913-919, doi:10.1016/j.bbabio.2007.05.005 (2007).
74. Mattiazzi, M. et al. The mtDNA T8993G (NARP) mutation results in an impairment of oxidative phosphorylation that can be improved by antioxidants. Human molecular genetics 13, 869-879, doi:10.1093/hmg/ddh 103 (2004).
75. Rogatzki, M. J., Ferguson, B. S., Goodwin, M. L. & Gladden, L. B. Lactate is always the end product of glycolysis. Frontiers in neuroscience 9, doi:Artn 22
76. 10.3389/Fnins.2015.00022 (2015).
77. Licznerski, P. et al. ATP Synthase c-Subunit Leak Causes Aberrant Cellular Metabolism in Fragile X Syndrome. Cell 182, 1170-+, doi:10.1016/j.cell.2020.07.008 (2020).
78. Hong, S. & Pedersen, P. L. ATP synthase and the actions of inhibitors utilized to study its roles in human health, disease, and other scientific areas. Microbiology and molecular biology reviews: MMBR 72, 590-641, Table of Contents, doi:10.1128/MMBR.00016-08 (2008).
79. Schulenburg, H. & Boehnisch, C. Diversification and adaptive sequence evolution of Caenorhabditis lysozymes (Nematoda: Rhabditidae). BMC evolutionary biology 8, 114, doi:10.1186/1471-2148-8-114 (2008).
80. Goodell, E. W. & Schwarz, U. Release of cell wall peptides into culture medium by exponentially growing Escherichia coli. Journal of bacteriology 162, 391-397, doi:10.1128/JB.162.1.391-397.1985 (1985).
81. Park, J. T. & Uehara, T. How bacteria consume their own exoskeletons (turnover and recycling of cell wall peptidoglycan). Microbiology and molecular biology reviews: MMBR 72, 211-227, table of contents, doi:10.1128/MMBR.00027-07 (2008).
82. Lapuente-Brun, E. et al. Supercomplex assembly determines electron flux in the mitochondrial electron transport chain. Science 340, 1567-1570, doi:10.1126/science.1230381 (2013).
83. Brenner, S. The genetics of Caenorhabditis elegans. Genetics 77, 71-94 (1974).
84. Juan, C., Torrens, G., Barcelo, I. M. & Oliver, A. Interplay between Peptidoglycan Biology and Virulence in Gram-Negative Pathogens. Microbiology and molecular biology reviews: MMBR 82, doi:10.1128/MMBR.00033-18 (2018).
85. Typas, A., Banzhaf, M., Gross, C. A. & Vollmer, W. From the regulation of peptidoglycan synthesis to bacterial growth and morphology. Nature reviews. Microbiology 10, 123-136, doi:10.1038/nrmicro2677 (2011).
86. van Heijenoort, J. Peptidoglycan hydrolases of Escherichia coli. Microbiology and molecular biology reviews: MMBR 75, 636-663, doi:10.1128/MMBR.00022-11 (2011).
87. Vollmer, W. & Holtje, J. V. The architecture of the murein (peptidoglycan) in gram-negative bacteria: vertical scaffold or horizontal layer(s)? Journal of bacteriology 186, 5978-5987, doi:10.1128/JB.186.18.5978-5987.2004 (2004).
88. MacNeil, L. T., Watson, E., Arda, H. E., Zhu, L. J. & Walhout, A. J. Diet-induced developmental acceleration independent of TOR and insulin in C. elegans. Cell 153, 240-252, doi:10.1016/j.cell.2013.02.049 (2013).
89. Coppa, A. et al. The peroxisomal fatty acid transporter ABCD1/PMP-4 is required in the C. elegans hypodermis for axonal maintenance: A worm model for adrenoleukodystrophy. Free radical biology & medicine 152, 797-809, doi:10.1016/j.freeradbiomed.2020.01.177 (2020).
90. Dunbar, T. L., Yan, Z., Balla, K. M., Smelkinson, M. G. & Troemel, E. R. C. elegans detects pathogen-induced translational inhibition to activate immune signaling. Cell host & microbe 11, 375-386, doi:10.1016/j.chom.2012.02.008 (2012).
91. Bolz, D. D., Tenor, J. L. & Aballay, A. A conserved PMK-1/p38 MAPK is required in Caenorhabditis elegans tissue-specific immune response to Yersinia pestis infection. The Journal of biological chemistry 285, 10832-10840, doi:10.1074/jbc.M109.091629 (2010).
92. Sanders, A. N. & Pavelka, M. S. Phenotypic analysis of Escherichia coli mutants lacking L,D-transpeptidases. Microbiology 159, 1842-1852, doi:10.1099/mic.0.069211-0 (2013).
93. Magnet, S. et al. Identification of the L,D-transpeptidases responsible for attachment of the Braun lipoprotein to Escherichia coli peptidoglycan. Journal of bacteriology 189, 3927-3931, doi:10.1128/JB.00084-07 (2007).
94. Uehara, T., Dinh, T. & Bernhardt, T. G. LytM-domain factors are required for daughter cell separation and rapid ampicillin-induced lysis in Escherichia coli. Journal of bacteriology 191, 5094-5107, doi:10.1128/JB.00505-09 (2009).
95. Alavian, K. N., Li, H., Collis, L., Bonanni, L., Zeng, L., Sacchetti, S., Lazrove, E., Nabili, P., Flaherty, B., Graham, M., et al. (2011). Bcl-xL regulates metabolic efficiency of neurons through interaction with the mitochondrial FIFO ATP synthase. Nature cell biology 13, 1224-1233. 10.1038/ncb2330.
96. Baracca, A., Sgarbi, G., Mattiazzi, M., Casalena, G., Pagnotta, E., Valentino, M. L., Moggio, M., Lenaz, G., Carelli, V., and Solaini, G. (2007). Biochemical phenotypes associated with the mitochondrial ATP6 gene mutations at nt8993. Biochimica et biophysica acta 1767, 913-919. 10.1016/j.bbabio.2007.05.005.
97. Chang, Y., Hsieh, P. H., and Chao, C. C. (2009). The efficiency of Percoll and Ficoll density gradient media in the isolation of marrow derived human mesenchymal stem cells with osteogenic potential. Chang Gung Med J 32, 264-275.
98. Chin, R. M., Fu, X., Pai, M. Y., Vergnes, L., Hwang, H., Deng, G., Diep, S., Lomenick, B., Meli, V. S., Monsalve, G. C., et al. (2014). The metabolite alpha-ketoglutarate extends lifespan by inhibiting ATP synthase and TOR. Nature 510, 397-401. 10.1038/nature13264.
99. Dautant, A., Meier, T., Hahn, A., Tribouillard-Tanvier, D., di Rago, J. P., and Kucharczyk, R. (2018). ATP Synthase Diseases of Mitochondrial Genetic Origin. Front Physiol 9, 329. 10.3389/fphys.2018.00329.
100. Demers-Lamarche, J., Guillebaud, G., Tlili, M., Todkar, K., Belanger, N., Grondin, M.,
101. Nguyen, A. P., Michel, J., and Germain, M. (2016). Loss of Mitochondrial Function Impairs Lysosomes. J Biol Chem 291, 10263-10276. 10.1074/jbc.M115.695825.
102. Dingley, S., Polyak, E., Lightfoot, R., Ostrovsky, J., Rao, M., Greco, T., Ischiropoulos, H., and Falk, M. J. (2010). Mitochondrial respiratory chain dysfunction variably increases oxidant stress in Caenorhabditis elegans. Mitochondrion 10, 125-136. 10.1016/j.mito.2009.11.003.
103. Esparza-Molto, P. B., and Cuezva, J. M. (2018). The Role of Mitochondrial H (+)-ATP Synthase in Cancer. Frontiers in oncology 8, 53. 10.3389/fonc.2018.00053.
104. Girardin, S. E., Boneca, I. G., Carneiro, L. A., Antignac, A., Jehanno, M., Viala, J., Tedin, K., Taha, M. K., Labigne, A., Zahringer, U., et al. (2003). Nodl detects a unique muropeptide from gram-negative bacterial peptidoglycan. Science 300, 1584-1587. 10.1126/science.1084677.
105. Glauner, B., Holtje, J. V., and Schwarz, U. (1988). The composition of the murein of Escherichia coli. J Biol Chem 263, 10088-10095.
106. Goldberg, J., Currais, A., Prior, M., Fischer, W., Chiruta, C., Ratliff, E., Daugherty, D., Dargusch, R., Finley, K., Esparza-Molto, P. B., et al. (2018). The mitochondrial ATP synthase is a shared drug target for aging and dementia. Aging cell 17. 10.1111/acel.12715.
107. Gottar, M., Gobert, V., Michel, T., Belvin, M., Duyk, G., Hoffmann, J. A., Ferrandon, D., and Royet, J. (2002). The Drosophila immune response against Gram-negative bacteria is mediated by a peptidoglycan recognition protein. Nature 416, 640-644. 10.1038/nature734.
108. Guillouzo, A., and Guguen-Guillouzo, C. (2020). Antibiotics-induced oxidative stress. Curr Opin Toxicol 20-21, 23-28. 10.1016/j.cotox.2020.03.004.
109. Henriques, M., Diogo, L., Garcia, P., Pratas, J., Simoes, M., and Grazina, M. (2012). Mitochondrial DNA 8993T>G mutation in a child with ornithine transcarbamylase deficiency and leigh syndrome: an unexpected association. Journal of child neurology 27, 1059-1061. 10.1177/0883073811431015.
Hong, S., and Pedersen, P. L. (2008). ATP synthase and the actions of inhibitors utilized to study its roles in human health, disease, and other scientific areas. Microbiology and molecular biology reviews: MMBR 72, 590-641, Table of Contents. 10.1128/MMBR.00016-08.
110. Humann, J., and Lenz, L. L. (2009). Bacterial peptidoglycan degrading enzymes and their impact on host muropeptide detection. Journal of innate immunity 1, 88-97. 10.1159/000181181.
111. Iannuzzi, F., Frisardi, V., Annunziato, L., and Matrone, C. (2021). Might Fibroblasts from Patients with Alzheimer's Disease Reflect the Brain Pathology? A Focus on the Increased Phosphorylation of Amyloid Precursor Protein Tyr682 Residue. Brain Sci 11. 10.3390/brainsci11010103.
112. Irazoki, O., Hernandez, S. B., and Cava, F. (2019). Peptidoglycan Muropeptides: Release, Perception, and Functions as Signaling Molecules. Front Microbiol 10, 500. 10.3389/fmicb.2019.00500.
113. Jauslin, M. L., Meier, T., Smith, R. A., and Murphy, M. P. (2003). Mitochondria-targeted antioxidants protect Friedreich Ataxia fibroblasts from endogenous oxidative stress more effectively than untargeted antioxidants. Faseb J 17, 1972-1974. 10.1096/fj.03-0240fje.
114. Jauslin, M. L., Wirth, T., Meier, T., and Schoumacher, F. (2002). A cellular model for Friedreich Ataxia reveals small-molecule glutathione peroxidase mimetics as novel treatment strategy. Hum Mol Genet 11, 3055-3063. 10.1093/hmg/11.24.3055.
115. Kalghatgi, S., Spina, C. S., Costello, J. C., Liesa, M., Morones-Ramirez, J. R., Slomovic, S., Molina, A., Shirihai, O. S., and Collins, J. J. (2013). Bactericidal antibiotics induce mitochondrial dysfunction and oxidative damage in Mammalian cells. Sci Transl Med 5, 192ra185. 10.1126/scitranslmed.3006055.
116. Kniazeva, M., Zhu, H., Sewell, A. K., and Han, M. (2015). A Lipid-TORC1 Pathway Promotes Neuronal Development and Foraging Behavior under Both Fed and Fasted Conditions in C. elegans. Dev Cell 33, 260-271. 10.1016/j.devcel.2015.02.015.
117. LaFauci, G., Adayev, T., Kascsak, R., and Brown, W. T. (2016). Detection and Quantification of the Fragile X Mental Retardation Protein 1 (FMRP). Genes (Basel) 7. 10.3390/genes7120121.
Lee, J., Giordano, S., and Zhang, J. (2012). Autophagy, mitochondria and oxidative stress: cross-talk and redox signalling. Biochem J 441, 523-540. 10.1042/BJ20111451.
118. Lee, S. J., Hwang, A. B., and Kenyon, C. (2010). Inhibition of respiration extends C. elegans life span via reactive oxygen species that increase HIF-1 activity. Current biology: CB 20, 2131-2136. 10.1016/j.cub.2010.10.057.
119. Liu, Y., Samuel, B. S., Breen, P. C., and Ruvkun, G. (2014). Caenorhabditis elegans pathways that surveil and defend mitochondria. Nature 508, 406-410. 10.1038/nature13204.
120. Magnet, S., Bellais, S., Dubost, L., Fourgeaud, M., Mainardi, J. L., Petit-Frere, S., Marie, A., Mengin-Lecreulx, D., Arthur, M., and Gutmann, L. (2007). Identification of the L,D-transpeptidases responsible for attachment of the Braun lipoprotein to Escherichia coli peptidoglycan. Journal of bacteriology 189, 3927-3931. 10.1128/JB.00084-07.
121. Mann, B., Loh, L. N., Gao, G., and Tuomanen, E. (2016). Preparation of Purified Gram-positive Bacterial Cell Wall and Detection in Placenta and Fetal Tissues. Bio Protoc 6. 10.21769/BioProtoc.2037.
122. Mattiazzi, M., Vijayvergiya, C., Gajewski, C. D., DeVivo, D. C., Lenaz, G., Wiedmann, M., and Manfredi, G. (2004). The mtDNA T8993G (NARP) mutation results in an impairment of oxidative phosphorylation that can be improved by antioxidants. Human molecular genetics 13, 869-879. 10.1093/hmg/ddh103.
123. McDonald, K., Schwarz, H., Muller-Reichert, T., Webb, R., Buser, C., and Morphew, M. (2010). “Tips and tricks” for high-pressure freezing of model systems. Methods Cell Biol 96, 671-693. 10.1016/S0091-679X (10) 96028-7.
124. Melo, J. A., and Ruvkun, G. (2012). Inactivation of conserved C. elegans genes engages pathogen- and xenobiotic-associated defenses. Cell 149, 452-466. 10.1016/j.cell.2012.02.050.
125. Nee, L. E., Polinsky, R. J., Eldridge, R., Weingartner, H., Smallberg, S., and Ebert, M. (1983). A family with histologically confirmed Alzheimer's disease. Arch Neurol 40, 203-208. 10.1001/archneur.1983.04050040033004.
126. Ortiz-Gonzalez, X. R. (2021). Mitochondrial Dysfunction: A Common Denominator in Neurodevelopmental Disorders? Dev Neurosci 43, 222-229. 10.1159/000517870.
127. Palikaras, K., Lionaki, E., and Tavernarakis, N. (2018). Mechanisms of mitophagy in cellular homeostasis, physiology and pathology. Nat Cell Biol 20, 1013-1022. 10.1038/s41556-018-0176-2.
128. Pastores, G. M., Santorelli, F. M., Shanske, S., Gelb, B. D., Fyfe, B., Wolfe, D., and Willner, J. P. (1994). Leigh syndrome and hypertrophic cardiomyopathy in an infant with a mitochondrial DNA point mutation (T8993G). American journal of medical genetics 50, 265-271. 10.1002/ajmg. 1320500310.
129. Rogatzki, M. J., Ferguson, B. S., Goodwin, M. L., and Gladden, L. B. (2015). Lactate is always the end product of glycolysis. Frontiers in neuroscience 9. Artn 22 10.3389/Fnins.2015.00022.
130. Royet, J., Gupta, D., and Dziarski, R. (2011). Peptidoglycan recognition proteins: modulators of the microbiome and inflammation. Nat Rev Immunol 11, 837-851. 10.1038/nri3089.
131. Sanders, A. N., and Pavelka, M. S. (2013). Phenotypic analysis of Escherichia coli mutants lacking L,D-transpeptidases. Microbiology 159, 1842-1852. 10.1099/mic.0.069211-0.
132. Schniertshauer, D., Gebhard, D., and Bergemann, J. (2018). Age-Dependent Loss of Mitochondrial Function in Epithelial Tissue Can Be Reversed by Coenzyme Q10. J Aging Res 2018, 6354680. 10.1155/2018/6354680.
133. Typas, A., Banzhaf, M., Gross, C. A., and Vollmer, W. (2011). From the regulation of peptidoglycan synthesis to bacterial growth and morphology. Nat Rev Microbiol 10, 123-136. 10.1038/nrmicro2677.
134. Uehara, T., Dinh, T., and Bernhardt, T. G. (2009). LytM-domain factors are required for daughter cell separation and rapid ampicillin-induced lysis in Escherichia coli. Journal of bacteriology 191, 5094-5107. 10.1128/JB.00505-09.
135. Wu, D., and Yotnda, P. (2011). Production and detection of reactive oxygen species (ROS) in cancers. J Vis Exp. 10.3791/3357.
136. Zarrinpar, A., Chaix, A., Xu, Z. Z., Chang, M. W., Marotz, C. A., Saghatelian, A., Knight, R., and Panda, S. (2018). Antibiotic-induced microbiome depletion alters metabolic homeostasis by affecting gut signaling and colonic metabolism. Nat Commun 9, 2872. 10.1038/s41467-018-05336-9.
137. Zhang, J., Li, X., Olmedo, M., Holdorf, A. D., Shang, Y., Artal-Sanz, M., Yilmaz, L. S., and Walhout, A. J. M. (2019). A Delicate Balance between Bacterial Iron and Reactive Oxygen Species Supports Optimal C. elegans Development. Cell host & microbe 26, 400-411 e403. 10.1016/j.chom.2019.07.010.
138. Beal, M. F. (2005). Oxidative damage as an early marker of Alzheimer's disease and mild cognitive impairment. Neurobiol Aging 26, 585-586. 10.1016/j.neurobiolaging.2004.09.022.
139. Beilina, A., and Cookson, M. R. (2016). Genes associated with Parkinson's disease: regulation of autophagy and beyond. J Neurochem 139 Suppl 1, 91-107. 10.1111/jnc.13266.
140. Benchoua, A., Trioulier, Y., Zala, D., Gaillard, M. C., Lefort, N., Dufour, N., Saudou, F., Elalouf, J. M., Hirsch, E., Hantraye, P., et al. (2006). Involvement of mitochondrial complex II defects in neuronal death produced by N-terminus fragment of mutated huntingtin. Mol Biol Cell 17, 1652-1663. 10.1091/mbc.e05-07-0607.
141. Boudina, S., Sena, S., Theobald, H., Sheng, X., Wright, J. J., Hu, X. X., Aziz, S., Johnson, J. I., Bugger, H., Zaha, V. G., and Abel, E. D. (2007). Mitochondrial energetics in the heart in obesity-related diabetes: direct evidence for increased uncoupled respiration and activation of uncoupling proteins. Diabetes 56, 2457-2466. 10.2337/db07-0481.
142. Brouillet, E., Hantraye, P., Ferrante, R. J., Dolan, R., Leroy-Willig, A., Kowall, N. W., and Beal, M. F. (1995). Chronic mitochondrial energy impairment produces selective striatal degeneration and abnormal choreiform movements in primates. Proc Natl Acad Sci USA 92, 7105-7109. 10.1073/pnas.92.15.7105.
143. Cenini, G., and Voos, W. (2019). Mitochondria as Potential Targets in Alzheimer Disease Therapy: An Update. Front Pharmacol 10, 902. 10.3389/fphar.2019.00902.
144. Chouchani, E. T., Pell, V. R., Gaude, E., Aksentijevic, D., Sundier, S. Y., Robb, E. L., Logan, A., Nadtochiy, S. M., Ord, E. N. J., Smith, A. C., et al. (2014). Ischaemic accumulation of succinate controls reperfusion injury through mitochondrial ROS. Nature 515, 431-435. 10.1038/nature13909.
145. Dautant, A., Meier, T., Hahn, A., Tribouillard-Tanvier, D., di Rago, J. P., and Kucharczyk, R. (2018). ATP Synthase Diseases of Mitochondrial Genetic Origin. Front Physiol 9, 329. 10.3389/fphys.2018.00329.
146 Fassone, E., Taanman, J. W., Hargreaves, I. P., Sebire, N. J., Cleary, M. A., Burch, M., and Rahman, S. (2011). Mutations in the mitochondrial complex I assembly factor NDUFAF1 cause fatal infantile hypertrophic cardiomyopathy. J Med Genet 48, 691-697. 10.1136/jmedgenet-2011-100340.
147. Finsterer, J., and Zarrouk Mahjoub, S. (2012). Epilepsy in mitochondrial disorders. Seizure 21, 316-321. 10.1016/j.seizure.2012.03.003.
148. Frye, R. E. (2020). Mitochondrial Dysfunction in Autism Spectrum Disorder: Unique Abnormalities and Targeted Treatments. Semin Pediatr Neurol 35, 100829. 10.1016/j.spen.2020.100829.
149. Gonzalez-Rodriguez, P., Zampese, E., Stout, K. A., Guzman, J. N., Ilijic, E., Yang, B., Tkatch, T., Stavarache, M. A., Wokosin, D. L., Gao, L., et al. (2021). Disruption of mitochondrial complex I induces progressive parkinsonism. Nature 599, 650-656. 10.1038/s41586-021-04059-0.
150. Grace, P. M., Gaudet, A. D., Staikopoulos, V., Maier, S. F., Hutchinson, M. R., Salvemini, D., and Watkins, L. R. (2016). Nitroxidative Signaling Mechanisms in Pathological Pain. Trends Neurosci 39, 862-879. 10.1016/j.tins.2016.10.003.
151. Guillouzo, A., and Guguen-Guillouzo, C. (2020). Antibiotics-induced oxidative stress. Curr Opin Toxicol 20-21, 23-28. 10.1016/j.cotox.2020.03.004.
152. Haelterman, N. A., Yoon, W. H., Sandoval, H., Jaiswal, M., Shulman, J. M., and Bellen, H. J. (2014). A mitocentric view of Parkinson's disease. Annu Rev Neurosci 37, 137-159. 10.1146/annurev-neuro-071013-014317.
153. James, A. M., Collins, Y., Logan, A., and Murphy, M. P. (2012). Mitochondrial oxidative stress and the metabolic syndrome. Trends Endocrinol Metab 23, 429-434. 10.1016/j.tem.2012.06.008.
154. Johnson, J., Mercado-Ayon, E., Mercado-Ayon, Y., Dong, Y. N., Halawani, S., Ngaba, L., and Lynch, D. R. (2021). Mitochondrial dysfunction in the development and progression of neurodegenerative diseases. Arch Biochem Biophys 702, 108698. 10.1016/j.abb.2020.108698.
155. Kalghatgi, S., Spina, C. S., Costello, J. C., Liesa, M., Morones-Ramirez, J. R., Slomovic, S., Molina, A., Shirihai, O. S., and Collins, J. J. (2013). Bactericidal antibiotics induce mitochondrial dysfunction and oxidative damage in Mammalian cells. Sci Transl Med 5, 192ra185. 10.1126/scitranslmed.3006055.
156. Lin, M. T., and Beal, M. F. (2006). Mitochondrial dysfunction and oxidative stress in neurodegenerative diseases. Nature 443, 787-795. 10.1038/nature05292.
157. Lott, M. T., Leipzig, J. N., Derbeneva, O., Xie, H. M., Chalkia, D., Sarmady, M., Procaccio, V., and Wallace, D. C. (2013). mtDNA Variation and Analysis Using Mitomap and Mitomaster. Curr Protoc Bioinformatics 44, 1 23 21-26. 10.1002/0471250953.bi0123s44.
158. Lowes, D. A., Wallace, C., Murphy, M. P., Webster, N. R., and Galley, H. F. (2009). The mitochondria targeted antioxidant MitoQ protects against fluoroquinolone-induced oxidative stress and mitochondrial membrane damage in human Achilles tendon cells. Free Radic Res 43, 323-328. 10.1080/10715760902736275.
159. Mahalaxmi, I., Subramaniam, M. D., Gopalakrishnan, A. V., and Vellingiri, B. (2021). Dysfunction in Mitochondrial Electron Transport Chain Complex I, Pyruvate Dehydrogenase Activity, and Mutations in ND1 and ND4 Gene in Autism Spectrum Disorder Subjects from Tamil Nadu Population, India. Mol Neurobiol 58, 5303-5311. 10.1007/s12035-021-02492-w.
160. McKee, E. E., Ferguson, M., Bentley, A. T., and Marks, T. A. (2006). Inhibition of mammalian mitochondrial protein synthesis by oxazolidinones. Antimicrob Agents Chemother 50, 2042-2049. 10.1128/AAC.01411-05.
161. Milakovic, T., and Johnson, G. V. (2005). Mitochondrial respiration and ATP production are significantly impaired in striatal cells expressing mutant huntingtin. J Biol Chem 280, 30773-30782. 10.1074/jbc.M504749200.
162. Mochel, F., and Haller, R. G. (2011). Energy deficit in Huntington disease: why it matters. J Clin Invest 121, 493-499. 10.1172/JCI45691.
163. Montgomery, M. K., and Turner, N. (2015). Mitochondrial dysfunction and insulin resistance: an update. Endocr Connect 4, R1-R15. 10.1530/EC-14-0092.
164. Nunomura, A., Perry, G., Aliev, G., Hirai, K., Takeda, A., Balraj, E. K., Jones, P. K., Ghanbari, H., Wataya, T., Shimohama, S., et al. (2001). Oxidative damage is the earliest event in Alzheimer disease. J Neuropathol Exp Neurol 60, 759-767. 10.1093/jnen/60.8.759.
165. Ortiz-Gonzalez, X. R. (2021). Mitochondrial Dysfunction: A Common Denominator in Neurodevelopmental Disorders? Dev Neurosci 43, 222-229. 10.1159/000517870.
166. Prasuhn, J., Davis, R. L., and Kumar, K. R. (2020). Targeting Mitochondrial Impairment in Parkinson's Disease: Challenges and Opportunities. Front Cell Dev Biol 8, 615461. 10.3389/fcell.2020.615461.
167. Prasun, P. (2020). Mitochondrial dysfunction in metabolic syndrome. Biochim Biophys Acta Mol Basis Dis 1866, 165838. 10.1016/j.bbadis.2020.165838.
168. Pritam, P., Deka, R., Bhardwaj, A., Srivastava, R., Kumar, D., Jha, A. K., Jha, N. K., Villa, C., and Jha, S. K. (2022). Antioxidants in Alzheimer's Disease: Current Therapeutic Significance and Future Prospects. Biology (Basel) 11. 10.3390/biology11020212.
169. Rahman, S. (2012). Mitochondrial disease and epilepsy. Dev Med Child Neurol 54, 397-406. 10.1111/j. 1469-8749.2011.04214.x.
170. Reddy, P. H., Tripathi, R., Troung, Q., Tirumala, K., Reddy, T. P., Anekonda, V., Shirendeb, U. P., Calkins, M. J., Reddy, A. P., Mao, P., and Manczak, M. (2012). Abnormal mitochondrial dynamics and synaptic degeneration as early events in Alzheimer's disease: implications to mitochondria-targeted antioxidant therapeutics. Biochim Biophys Acta 1822, 639-649. 10.1016/j.bbadis.2011.10.011.
171. Sawaddiruk, P., Apaijai, N., Paiboonworachat, S., Kaewchur, T., Kasitanon, N., Jaiwongkam, T., Kerdphoo, S., Chattipakorn, N., and Chattipakorn, S. C. (2019). Coenzyme Q10 supplementation alleviates pain in pregabalin-treated fibromyalgia patients via reducing brain activity and mitochondrial dysfunction. Free Radic Res 53, 901-909. 10.1080/10715762.2019.1645955.
172. Skladal, D., Halliday, J., and Thorburn, D. R. (2003). Minimum birth prevalence of mitochondrial respiratory chain disorders in children. Brain 126, 1905-1912. 10.1093/brain/awg170.
173. Stewart, J. B., and Chinnery, P. F. (2015). The dynamics of mitochondrial DNA heteroplasmy: implications for human health and disease. Nat Rev Genet 16, 530-542. 10.1038/nrg3966.
174. Surmeier, D. J., Obeso, J. A., and Halliday, G. M. (2017). Selective neuronal vulnerability in Parkinson disease. Nat Rev Neurosci 18, 101-113. 10.1038/nrn.2016.178.
175. Tragni, V., Primiano, G., Tummolo, A., Cafferati Beltrame, L., La Piana, G., Sgobba, M. N., Cavalluzzi, M. M., Paterno, G., Gorgoglione, R., Volpicella, M., et al. (2022). Personalized Medicine in Mitochondrial Health and Disease: Molecular Basis of Therapeutic Approaches Based on Nutritional Supplements and Their Analogs. Molecules 27. 10.3390/molecules27113494.
176. Tzoulis, C., Tran, G. T., Coxhead, J., Bertelsen, B., Lilleng, P. K., Balafkan, N., Payne, B., Miletic, H., Chinnery, P. F., and Bindoff, L. A. (2014). Molecular pathogenesis of polymerase gamma-related neurodegeneration. Ann Neurol 76, 66-81. 10.1002/ana.24185.
177. Valiente-Palleja, A., Torrell, H., Muntane, G., Cortes, M. J., Martinez-Leal, R., Abasolo, N., Alonso, Y., Vilella, E., and Martorell, L. (2018). Genetic and clinical evidence of mitochondrial dysfunction in autism spectrum disorder and intellectual disability. Hum Mol Genet 27, 891-900. 10.1093/hmg/ddy009.
178 Vitek, M. P., Araujo, J. A., Fossel, M., Greenberg, B. D., Howell, G. R., Rizzo, S. J. S., Seyfried, N. T., Tenner, A. J., Territo, P. R., Windisch, M., et al. (2020). Translational animal models for Alzheimer's disease: An Alzheimer's Association Business Consortium Think Tank. Alzheimers Dement (NY) 6, e12114. 10.1002/trc2.12114.
179. Yang, N., Guan, Q. W., Chen, F. H., Xia, Q. X., Yin, X. X., Zhou, H. H., and Mao, X. Y. (2020). Antioxidants Targeting Mitochondrial Oxidative Stress: Promising Neuroprotectants for Epilepsy. Oxid Med Cell Longev 2020, 6687185. 10.1155/2020/6687185.
180. Zhou, B., and Tian, R. (2018). Mitochondrial dysfunction in pathophysiology of heart failure. J Clin Invest 128, 3716-3726. 10.1172/JCI120849.

What is claimed is:

1. A method of treating a mitochondrial disease, the method comprising administering a therapeutically effective amount of isolated therapeutic peptidoglycan (PG) muropeptide.

2. The method of claim 1, wherein said therapeutic PG muropeptide is generated by treating a PG molecule with a lysozyme, or synthesized in vitro.

3. The method of any of claims 1 and 2, wherein said therapeutic PG muropeptide is generated from a PG that is not associated with a lipoprotein.

4. The method of any of claims 1-3, wherein said therapeutic PG muropeptide comprises a 5′ N-acetylglucosamine (NAG)-N-acetylmuramic acid (NAM) disaccharide muropeptides with an amino acid peptide attached to said NAM.

5. The method of claim 1, wherein said therapeutic PG muropeptide are selected from the group consisting of: a muropeptides dimer, a muropeptides oligomer, and a combination of the same.

6. The method of claim 1, wherein said therapeutic PG muropeptide interacts with, and/or stabilizes at least one subunit of the ATP synthase complex in the subject.

7. The method of claim 6, wherein said at least one ATP synthase subunit is selected from the group consisting of: the α subunit, the d subunit, the F subunit, or a combination of the same.

8. The method of claim 1, wherein said therapeutic PG muropeptide acts as an ATP synthase agonist thereby increasing ATP synthase activity in the subject.

9. The method of claim 1, wherein said therapeutic PG muropeptide inhibits formation of reactive oxygen species (ROS) in the subject.

10. The method of claim 1, wherein said therapeutic PG muropeptide inhibits mitochondrial oxidative stress in the subject.

11. The method of claim 1, wherein said mitochondrial disease is selected from the group consisting of: Apical hypertrophic cardiomyopathy (AHCM), neuropathy, ataxia, autism, Charcot-Marie-Tooth syndrome (CMT), encephalopathy, epilepsy with brain pseudoatrophy, Episodic Weakness, Hereditary Spastic Paraplegia (HSP), Familiar Bilateral Striatal Necrosis (FBSN), Infantile cardiomyopathy, Leber Hereditary Optic neuropathy (LHON), Left Ventricular Hyper Trabeculation syndrome (LVHT), Maternally inherited Diabetes, Deafness syndrome (MIDD), Maternally inherited Leigh Syndrome (MILS), Mesial Temporal Lobe Epilepsies with Hippocampal Sclerosis (MTLE-HS), Metabolic Syndrome (MS), Motor Neuron Syndrome (MNS), Myopathy, lactic Acidosis, Sideroblastic Anemia (MLASA), Neurogenetic Ataxia Retinis Pigmentosa syndrome (NARP), Periodic paralyzes, Schizophrenia, Spino Cerebellar Ataxia (SCA), Tetralogy of Fallot (ToF), short-chain acyl-coA dehydrogenase deficiency (SCAD), medium-chain acyl-coA dehydrogenase deficiency (MCAD), long-chain acyl-coA dehydrogenase deficiency LCAD), chronic progressive ophthalmoplegia (CPEO), Pearson Syndrome, Barth Syndrome, Alpers Disease, Luft Disease, mitochondrial neurogastrointestinal encephalomyopathy (MNGIE), neuropathy, ataxia, retinal pigmentosa (NARP), myoclonic epilepsy, ragged red fibers (MERRF), mitochondrial encephalopathy, lactic acidosis, stroke-like episodes (MELAS), Alzheimer's diseases (AD), Parkinson's disease (PD), Huntington's disease (HD), amyotrophic lateral sclerosis (ALS, aka Lou Gehring's diseases, Epilepsy, Autism, Fibromyalgia, chronic fatigue, cerebral palsy, Friedreich's Ataxia, Rett Syndrome, and Fragile X Syndrome, cardiomyopathy and muscular dystrophy (MD), diabetes and side effects of antibiotics treatment of bacterial infection.

12. The method of any of claims 1-11, wherein said subject in need thereof comprises a human subject.

13. The method of claim 1, wherein said pharmaceutically acceptable carrier comprises a nutritional supplement.

14. A pharmaceutical composition for the treatment of a mitochondrial disease in a subject in need thereof, comprising a therapeutically effective amount of isolated therapeutic PG muropeptide, and a pharmaceutically acceptable carrier.

15. The composition of claim 14, wherein said therapeutic PG muropeptide is generated by treating a PG molecule treated with a lysozyme, or synthesized in vitro.

16. The composition of any of claims 14 and 15, wherein said therapeutic PG muropeptide is generated from a PG that is not associated with a lipoprotein.

17. The composition of any of claim 16, wherein said therapeutic PG muropeptide comprises a 5′ N-acetylglucosamine (NAG)-N-acetylmuramic acid (NAM) disaccharide muropeptides with an amino acid peptide attached to said NAM.

18. The composition of claim 14, wherein said therapeutic PG muropeptide are selected from the group consisting of: a muropeptides dimer, a muropeptides oligomer, and a combination of the same.

19. The composition of claim 14, wherein said therapeutic PG muropeptide interacts with, and/or stabilizes at least one subunit of the ATP synthase complex in the subject.

20. The composition of claim 19, wherein said at least one ATP synthase subunit is selected from the group consisting of: the α subunit, the d subunit, the F subunit, or a combination of the same.

21. The composition of claim 14, wherein said therapeutic PG muropeptide acts as an ATP synthase agonist thereby increasing ATP synthase activity.

22. The composition of claim 14, wherein said therapeutic PG muropeptide inhibits formation of reactive oxygen species (ROS) in the subject.

23. The composition of claim 14, wherein said therapeutic PG muropeptide inhibits mitochondrial oxidative stress in the subject.

24. The composition of claim 14, wherein said mitochondrial disease is selected from the group consisting of: Apical hypertrophic cardiomyopathy (AHCM), neuropathy, ataxia, autism, Charcot-Marie-Tooth syndrome (CMT), encephalopathy, epilepsy with brain pseudoatrophy, Episodic Weakness, Hereditary Spastic Paraplegia (HSP), Familiar Bilateral Striatal Necrosis (FBSN), Infantile cardiomyopathy, Leber Hereditary Optic neuropathy (LHON), Left Ventricular Hyper Trabeculation syndrome (LVHT), Maternally inherited Diabetes, Deafness syndrome (MIDD), Maternally inherited Leigh Syndrome (MILS), Mesial Temporal Lobe Epilepsies with Hippocampal Sclerosis (MTLE-HS), Metabolic Syndrome (MS), Motor Neuron Syndrome (MNS), Myopathy, lactic Acidosis, Sideroblastic Anemia (MLASA), Neurogenetic Ataxia Retinis Pigmentosa syndrome (NARP), Periodic paralyzes, Schizophrenia, Spino Cerebellar Ataxia (SCA), Tetralogy of Fallot (ToF), short-chain acyl-coA dehydrogenase deficiency (SCAD), medium-chain acyl-coA dehydrogenase deficiency (MCAD), long-chain acyl-coA dehydrogenase deficiency LCAD), chronic progressive ophthalmoplegia (CPEO), Pearson Syndrome, Barth Syndrome, Alpers Disease, Luft Disease, mitochondrial neurogastrointestinal encephalomyopathy (MNGIE), neuropathy, ataxia, retinal pigmentosa (NARP), myoclonic epilepsy, ragged red fibers (MERRF), mitochondrial encephalopathy, lactic acidosis, stroke-like episodes (MELAS), Alzheimer's diseases (AD), Parkinson's disease (PD), Huntington's disease (HD), amyotrophic lateral sclerosis (ALS, aka Lou Gehring's diseases, Epilepsy, Autism, Fibromyalgia, chronic fatigue, cerebral palsy, Friedreich's Ataxia, Rett Syndrome, and Fragile X Syndrome, cardiomyopathy and muscular dystrophy (MD), diabetes and side effects of antibiotics treatment of bacterial infection.

25. A method of treating a mitochondrial disease, the method comprising administering a therapeutically effective amount of the composition of claim 14 to a subject in need thereof, and wherein said subject is preferably a human subject.

26. The composition of claim 14, wherein said pharmaceutically acceptable carrier comprises a nutritional supplement.

27. A method of producing a therapeutic muropeptide comprising:
establishing a quantity of peptidoglycan (PG);
removing any lipoproteins associated with said PG;
treating said PG with a lysozyme generating a quantity of therapeutic PG muropeptides;
isolating said therapeutic PG muropeptides; and
optionally combining said therapeutic PG muropeptides with a pharmaceutically acceptable carrier.

28. The method of claim 27, wherein said therapeutic PG muropeptide comprises a 5′ N-acetylglucosamine (NAG)-N-acetylmuramic acid (NAM) disaccharide muropeptide with an amino acid peptide attached to said NAM.

29. The method of claim 27, wherein said therapeutic PG muropeptide are selected from the group consisting of: a muropeptides dimer, a muropeptides oligomer, and a combination of the same.

30. The method of claim 27, wherein said therapeutic PG muropeptide interacts with and/or stabilizes at least one subunit of the ATP synthase complex in a subject in need thereof.

31. The method of claim 30, wherein said at least one ATP synthase subunit is selected from the group consisting of: the α subunit, the d subunit, the F subunit, or a combination of the same.

32. The method of claim 27, wherein said therapeutic PG muropeptide acts as an ATP synthase agonist.

33. The method of claim 27, wherein said therapeutic PG muropeptide inhibits formation of reactive oxygen species (ROS) in a subject in need thereof.

34. The method of claim 27, wherein said therapeutic PG muropeptide inhibits mitochondrial oxidative stress in a subject in need thereof.

35. The method of claim 27, wherein said therapeutic PG muropeptide is used to treat a mitochondrial disease in a subject in need thereof, selected from the group consisting of: Apical hypertrophic cardiomyopathy (AHCM), neuropathy, ataxia, autism, Charcot-Marie-Tooth syndrome (CMT), encephalopathy, epilepsy with brain pseudoatrophy, Episodic Weakness, Hereditary Spastic Paraplegia (HSP), Familiar Bilateral Striatal Necrosis (FBSN), Infantile cardiomyopathy, Leber Hereditary Optic neuropathy (LHON), Left Ventricular Hyper Trabeculation syndrome (LVHT), Maternally inherited Diabetes, Deafness syndrome (MIDD), Maternally inherited Leigh Syndrome (MILS), Mesial Temporal Lobe Epilepsies with Hippocampal Sclerosis (MTLE-HS), Metabolic Syndrome (MS), Motor Neuron Syndrome (MNS), Myopathy, lactic Acidosis, Sideroblastic Anemia (MLASA), Neurogenetic Ataxia Retinis Pigmentosa syndrome (NARP), Periodic paralyzes, Schizophrenia, Spino Cerebellar Ataxia (SCA), Tetralogy of Fallot (ToF), short-chain acyl-coA dehydrogenase deficiency (SCAD), medium-chain acyl-coA dehydrogenase deficiency (MCAD), long-chain acyl-coA dehydrogenase deficiency LCAD), chronic progressive ophthalmoplegia (CPEO), Pearson Syndrome, Barth Syndrome, Alpers Disease, Luft Disease, mitochondrial neurogastrointestinal encephalomyopathy (MNGIE), neuropathy, ataxia, retinal pigmentosa (NARP), myoclonic epilepsy, ragged red fibers (MERRF), mitochondrial encephalopathy, lactic acidosis, stroke-like episodes (MELAS), Alzheimer's diseases (AD), Parkinson's disease (PD), Huntington's disease (HD), amyotrophic lateral sclerosis (ALS, aka Lou Gehring's diseases, Epilepsy, Autism, Fibromyalgia, chronic fatigue, cerebral palsy, Friedreich's Ataxia, Rett Syndrome, and Fragile X Syndrome, cardiomyopathy and muscular dystrophy (MD), diabetes and side effects of antibiotics treatment of bacterial infection.

36. The method of any of claims 30-36, wherein said subject in need thereof comprises a human subject.

37. The method of claim 27, wherein said pharmaceutically acceptable carrier comprises a nutritional supplement.

38. A method of treating a mitochondrial disease, the method comprising administering a therapeutically effective amount of isolated therapeutic muropeptide combined with a pharmaceutically acceptable carrier to a subject in need thereof, wherein said therapeutic muropeptide comprises a 5′ N-acetylglucosamine (NAG)-N-acetylmuramic acid (NAM) disaccharide muropeptides with an amino acid peptide attached to said NAM.

39. A pharmaceutical composition for the treatment of a mitochondrial disease, comprising a therapeutically effective amount of isolated therapeutic muropeptide combined with a pharmaceutically acceptable carrier to a subject in need thereof, wherein said therapeutic muropeptide comprises a 5′ N-acetylglucosamine (NAG)-N-acetylmuramic acid (NAM) disaccharide muropeptide with an amino acid peptide attached to said NAM.

40. A method of producing a therapeutic muropeptide comprising:
establishing a quantity of peptidoglycan (PG);
removing any lipoproteins associated with said PG;
treating said PG with a lysozyme generating a quantity of therapeutic muropeptides;
isolating said therapeutic muropeptides; and
optionally combining said therapeutic muropeptides with a pharmaceutically acceptable carrier; and
wherein said therapeutic muropeptide comprises a 5′ N-acetylglucosamine (NAG)-N-acetylmuramic acid (NAM) disaccharide muropeptide with an amino acid peptide attached to said NAM.

41. An ATP synthase agonist comprising an isolated therapeutic PG muropeptide.

42. The ATP synthase agonist of claim 41, wherein said therapeutic PG muropeptide comprises a 5′ N-acetylglucosamine (NAG)-N-acetylmuramic acid (NAM) muropeptide with an amino acid peptide attached to said NAM.

43. A method of stabilizing an ATP synthase complex comprising contacting one or more subunits of said ATP synthase complex with an isolated therapeutic muropeptide.

44. The method of claim 43, wherein said ATP synthase subunit selected from the group consisting of: the α subunit, the d subunit, the F subunit, or a combination of the same.

45. A method of increasing ATP production in an assay comprising contacting one or more subunits of said ATP synthase complex with an isolated therapeutic muropeptide.

46. The method of claim 45, ATP synthase subunit selected from the group consisting of: the α subunit, the d subunit, the F subunit, or a combination of the same.

47. An assay having increased ATP production comprising an in vitro or in vivo assay that requires ATP synthesis, and including a quantity of isolated therapeutic PG muropeptide.

48. The assay of claim 47, wherein said therapeutic muropeptide comprises a 5′ N-acetylglucosamine (NAG)-N-acetylmuramic acid (NAM) disaccharide muropeptide with an amino acid peptide attached to said NAM.

49. The assay of any of claims 47-48, wherein said assay comprises an assay kit.

50. The method or composition of any claim above, wherein said therapeutic muropeptide are derived from PG of a gram (+) positive or Gram (−) negative bacteria, or synthesized in vitro.

51. A method of treating a mitochondrial disease, the method comprising administering a therapeutically effective amount of an isolated complex mixture of therapeutic PG muropeptides combined with a pharmaceutically acceptable carrier to a subject in need thereof.

52. The method of claim 51, wherein said complex mixture of therapeutic muropeptides are generated by treating one or more peptidoglycan (PG) molecules with a lysozyme, or synthesized in vitro.

53. The method of any of claim 51 or 52, wherein said complex mixture of therapeutic muropeptides are generated from a PG that is not associated with a lipoprotein.

54. The method of any of claims 51-53, wherein said complex mixture of therapeutic muropeptides comprises at least one therapeutic muropeptide comprising a 5′ N-acetylglucosamine (NAG)-N-acetylmuramic acid (NAM) disaccharide muropeptide with an amino acid peptide attached to said NAM.

55. The method of claim 51, wherein said complex mixture of therapeutic muropeptides comprises a mixture of disaccharide muropeptides, muropeptide dimers, a muropeptide oligomers, multi-saccharide muropeptides, and/or non-therapeutic muropeptides, or a combination of the same.

56. The method of claim 51, wherein said complex mixture of therapeutic muropeptides interacts with at least one subunit of the ATP synthase complex in the subject, and preferably an ATP synthase subunit selected from the group consisting of: the α subunit, the d subunit, the F subunit, or a combination of the same.

57. The method of claim 51, wherein said complex mixture of therapeutic muropeptides binds to and stabilizes at least one subunit of the ATP synthase complex in the subject, and preferably an ATP synthase subunit selected from the group consisting of: the α subunit, the d subunit, the F subunit, or a combination of the same.

58. The method of claim 51, wherein said complex mixture of therapeutic muropeptides acts as an ATP synthase agonist in the subject thereby increasing ATP synthase activity in the subject.

59. The method of claim 51, wherein said complex mixture of therapeutic muropeptides inhibit formation of reactive oxygen species (ROS) in the subject.

60. The method of claim 51, wherein said complex mixture of therapeutic muropeptides inhibits mitochondrial oxidative stress in the subject.

61. The method of claim 51, wherein said mitochondrial disease is selected from the group consisting of: Apical hypertrophic cardiomyopathy (AHCM), neuropathy, ataxia, autism, Charcot-Marie-Tooth syndrome (CMT), encephalopathy, epilepsy with brain pseudoatrophy, Episodic Weakness, Hereditary Spastic Paraplegia (HSP), Familiar Bilateral Striatal Necrosis (FBSN), Infantile cardiomyopathy, Leber Hereditary Optic neuropathy (LHON), Left Ventricular Hyper Trabeculation syndrome (LVHT), Maternally inherited Diabetes, Deafness syndrome (MIDD), Maternally inherited Leigh Syndrome (MILS), Mesial Temporal Lobe Epilepsies with Hippocampal Sclerosis (MTLE-HS), Metabolic Syndrome (MS), Motor Neuron Syndrome (MNS), Myopathy, lactic Acidosis, Sideroblastic Anemia (MLASA), Neurogenetic Ataxia Retinis Pigmentosa syndrome (NARP), Periodic paralyzes, Schizophrenia, Spino Cerebellar Ataxia (SCA), Tetralogy of Fallot (ToF), short-chain acyl-coA dehydrogenase deficiency (SCAD), medium-chain acyl-coA dehydrogenase deficiency (MCAD), long-chain acyl-coA dehydrogenase deficiency LCAD), chronic progressive ophthalmoplegia (CPEO), Pearson Syndrome, Barth Syndrome, Alpers Disease, Luft Disease, mitochondrial neurogastrointestinal encephalomyopathy (MNGIE), neuropathy, ataxia, retinal pigmentosa (NARP), myoclonic epilepsy, ragged red fibers (MERRF), mitochondrial encephalopathy, lactic acidosis, stroke-like episodes (MELAS), Alzheimer's diseases (AD), Parkinson's disease (PD), Huntington's disease (HD), amyotrophic lateral sclerosis (ALS, aka Lou Gehring's diseases, Epilepsy, Autism, Fibromyalgia, chronic fatigue, cerebral palsy, Friedreich's Ataxia, Rett Syndrome, and Fragile X Syndrome, cardiomyopathy and muscular dystrophy (MD), diabetes and side effects of antibiotics treatment of bacterial infection.

62. The method of any of claims 51-61, wherein said subject in need thereof comprises a human subject.

63. The method of any of claim 51-62, wherein said complex mixture of therapeutic muropeptides comprises an isolated complex mixture of therapeutic muropeptides.

64. A method of producing a therapeutic muropeptide comprising:
establishing a quantity of peptidoglycan (PG);
removing any lipoproteins associated with said PG;
treating said PG with a lysozyme generating a complex mixture of therapeutic muropeptides;
isolating said therapeutic muropeptides; and
optionally combining said therapeutic muropeptides with a pharmaceutically acceptable carrier.

65. The method of claim 64, wherein said complex mixture of therapeutic muropeptides comprises a least one therapeutic muropeptide having a 5′ N-acetylglucosamine (NAG)-N-acetylmuramic acid (NAM) disaccharide muropeptide with an amino acid peptide attached to said NAM.

66. The method of claim 64, wherein said complex mixture of therapeutic muropeptides comprises a mixture of disaccharide muropeptides, muropeptide dimers, a muropeptide oligomers, multi-saccharide muropeptides, and/or non-therapeutic muropeptides, or a combination of the same.

67. The method of claim 64, wherein said complex mixture of therapeutic muropeptides interacts with at least one subunit of the ATP synthase complex in a subject in need thereof, and preferably an ATP synthase subunit selected from the group consisting of: the α subunit, the d subunit, the F subunit, or a combination of the same.

68. The method of claim 64, wherein said complex mixture of therapeutic muropeptides binds to and stabilizes at least one subunit of the ATP synthase complex in a subject in need thereof, and preferably an ATP synthase subunit selected from the group consisting of: the α subunit, the d subunit, the F subunit, or a combination of the same.

69. The method of claim 64, wherein said complex mixture of therapeutic muropeptides acts as an ATP synthase agonist.

70. The method of claim 64, wherein said complex mixture of therapeutic muropeptides inhibit formation of reactive oxygen species (ROS) in a subject in need thereof.

71. The method of claim 64, wherein said complex mixture of therapeutic muropeptides inhibits mitochondrial oxidative stress in a subject in need thereof.

72. The method of claim 64, wherein said complex mixture of therapeutic muropeptides is used to treat a mitochondrial disease in a subject in need thereof, selected from the group consisting of: Alzheimer's disease, amyotrophic lateral sclerosis, Asperger's Disorder, Autistic Disorder, bipolar disorder, cancer, Cardiomyopathy, Charcot Marie Tooth disease (CMT, including subtypes such as CMT type 2b and 2b), Childhood Disintegrative Disorder (CDD), diabetes, epilepsy, Friedreich's Ataxia (FA), Hereditary motor and sensory neuropathy (HMSN), Huntington's Disease, Keams-Sayre Syndrome (KSS), Leber's Hereditary Optic Neuropathy (LHON, also referred to as Leber's Disease, Leber's Optic Atrophy (LOA), or Leber's Optic Neuropathy (LON)), Leigh Disease or Leigh Syndrome, macular degeneration, Mitochondrial Myopathy, Lactacidosis, and Stroke (MELAS), mitochondrial neurogastrointestinal encephalomyophathy (MNGIE), motor neuron diseases, Myoclonic Epilepsy With Ragged Red Fibers (MERRF), Neuropathy, ataxia, retinitis pigmentosa, and ptosis (NARP), Parkinson's disease, Peroneal muscular atrophy (PMA), Pervasive Developmental Disorder Not Otherwise Specified (PDD-NOS), renal tubular acidosis, Rett's Disorder, Schizophrenia, and strokes.

73. The method of claims 64-72, wherein said subject in need thereof comprises a human subject.

74. The method of claim 64, wherein said pharmaceutically acceptable carrier comprises a nutritional supplement.

75. An assay having increase ATP production comprising an in vitro or in vivo assay that requires ATP synthesis, and including a quantity of an isolated complex mixture of therapeutic muropeptides.

76. The assay of claim 75, wherein said complex mixture of therapeutic muropeptides comprises at least one therapeutic muropeptide 5′ N-acetylglucosamine (NAG)-N-acetylmuramic acid (NAM) disaccharide muropeptides with an amino acid peptide attached to said NAM.

77. The assay of claim 75-76, wherein said assay comprises an assay kit.

78. The method or composition of any claim above, wherein said therapeutic muropeptides are derived from PG of a gram (+) positive or Gram (−) negative bacteria, or synthesized in vitro.

79. A method of increasing the activity of ATP synthase in vitro or in a cell, the method comprising introducing an effective amount of a complex mixture of therapeutic muropeptides with an ATP synthase complex.

80. The method of claim 79, wherein said complex mixture of therapeutic muropeptides are generated by treating one or more peptidoglycan (PG) molecules with a lysozyme, or synthesized in vitro.

81. The method of any of claim 79 or 80, wherein said complex mixture of therapeutic muropeptides are generated from a PG that is not associated with a lipoprotein.

82. The method of any of claims 79-81, wherein said complex mixture of therapeutic muropeptides comprises at least one therapeutic muropeptide comprising a 5′ N-acetylglucosamine (NAG)-N-acetylmuramic acid (NAM) disaccharide muropeptide with an amino acid peptide attached to said NAM.

83. The method of claim 79, wherein said complex mixture of therapeutic muropeptides comprises a mixture of disaccharide muropeptides, muropeptide dimers, a muropeptide oligomers, non-therapeutic muropeptides, or a combination of the same.

84. The method of claim 79, wherein said complex mixture of therapeutic muropeptides interacts with and/or stabilize at least one subunit of the ATP synthase complex in the subject.

85. The method of claim 79, wherein said at least one synthase subunit is selected from the group consisting of: the α subunit, the d subunit, the F subunit, or a combination of the same.

86. The method of claim 79, wherein said complex mixture of therapeutic muropeptides acts as an ATP synthase agonist thereby increasing ATP synthase activity.

87. The method of claim 79, wherein said complex mixture of therapeutic muropeptides inhibit formation of reactive oxygen species (ROS) in the subject.

88. The method of claim 79, wherein said complex mixture of therapeutic muropeptides inhibits mitochondrial oxidative stress in the subject.

89. A method of increasing the activity of ATP synthase, the method comprising introducing an effective amount of a therapeutic PG muropeptide with an ATP synthase complex.

90. The method of claim 89, wherein said therapeutic muropeptide is generated by treating one or more peptidoglycan (PG) molecules with a lysozyme, or synthesized in vitro.

91. The method of any of claims 89 and 90, wherein said therapeutic muropeptide is generated from a PG that is not associated with a lipoprotein.

92. The method of any of claims 89-91, wherein said therapeutic muropeptide comprises at least one therapeutic muropeptide comprising a 5′ N-acetylglucosamine (NAG)-N-acetylmuramic acid (NAM) disaccharide muropeptide with an amino acid peptide attached to said NAM.

93. The method of claim 89, wherein said therapeutic PG muropeptide are selected from the group consisting of: a muropeptides dimer, a muropeptides oligomer, and a combination of the same.

94. The method of claim 89, wherein said therapeutic muropeptide interacts with and/or stabilizes to at least one subunit of the ATP synthase complex in the subject.

95. The method of claim 94, wherein said at least one ATP synthase subunit is selected from the group consisting of: the α subunit, the d subunit, the F subunit, or a combination of the same.

96. The method of claim 89, wherein said therapeutic muropeptide acts as an ATP synthase agonist.

97. The method of claim 89, wherein said therapeutic muropeptide inhibits formation of reactive oxygen species (ROS) in the subject.

98. The method of claim 89, wherein said therapeutic muropeptide inhibits mitochondrial oxidative stress in the subject.

99. A composition for increasing the activity of ATP synthase comprising an effective amount of an isolated therapeutic muropeptide, and optionally a pharmaceutically acceptable carrier.

100. The composition of claim 99, wherein said therapeutic muropeptide is generated by treating one or more peptidoglycan (PG) molecules with a lysozyme, or synthesized in vitro.

101. The composition of any of claim 99 or 10, wherein said therapeutic muropeptide is generated from a PG that is not associated with a lipoprotein.

102. The composition of any of claims 99-101, wherein said c therapeutic muropeptide comprises at least one therapeutic muropeptide comprising 5′ N-acetylglucosamine (NAG)-N-acetylmuramic acid (NAM) disaccharide muropeptide with an amino acid peptide attached to said NAM.

103. The composition of claim 99, wherein said therapeutic PG muropeptide are selected from the group consisting of: a muropeptides dimer, a muropeptides oligomer, and a combination of the same.

104. The composition of claim 99, wherein said therapeutic muropeptide interacts with and/or stabilizes at least one subunit of the ATP synthase complex in the subject.

105. The composition of claim 127, wherein said ATP synthase subunit is selected from the group consisting of: the α subunit, the d subunit, the F subunit, or a combination of the same.

106. The met composition of claim 12992, wherein said therapeutic muropeptide acts as an ATP synthase agonist.

107. The composition of claim 99, wherein said therapeutic muropeptide inhibits formation of reactive oxygen species (ROS).

108. The composition of claim 99, wherein said therapeutic muropeptide inhibits mitochondrial oxidative stress.

109. A method of increasing the Mitochondrial (Mt) oxidative respiration, the method comprising contacting an effective amount of an isolated therapeutic PG muropeptide, or a complex mixture of therapeutic PG muropeptides with a protein in the electron transport chain.

110. The method of claim 109, wherein said protein in the electron transport chain comprises UCR-1.

111. The method of claim 109, wherein said therapeutic muropeptide is generated by treating one or more peptidoglycan (PG) molecules with a lysozyme, or synthesized in vitro.

112. The method of any of claim 109 or 111, wherein said therapeutic PG muropeptide is generated from a PG that is not associated with a lipoprotein.

113. The method of any of claims 109-112, wherein said therapeutic PG muropeptide comprises at least one therapeutic muropeptide comprising a 5′ N-acetylglucosamine (NAG)-N-acetylmuramic acid (NAM) disaccharide muropeptide with an amino acid peptide attached to said NAM.

114. The method of claim 109, wherein said therapeutic PG muropeptide are selected from the group consisting of: a muropeptides dimer, a muropeptides oligomer, and a combination of the same.

115. The method of claim 109, wherein said therapeutic PG muropeptide inhibits formation of reactive oxygen species (ROS) in the subject.

116. The method of claim 109, wherein said therapeutic PG muropeptide inhibits mitochondrial oxidative stress in the subject.

117. A composition for increasing the Mitochondrial (Mt) oxidative respiration, comprising an effective amount of a therapeutic PG muropeptide, or a complex mixture of therapeutic muropeptides and a pharmaceutically acceptable carrier.

118. The compositions of claim 117, wherein said therapeutic PG muropeptide interacts with a UCR-1 protein.

119. The composition of claim 117, wherein said therapeutic PG muropeptide is generated by treating one or more peptidoglycan (PG) molecules with a lysozyme, or synthesized in vitro.

120. The composition of any of claim 117 or 119, wherein said therapeutic PG muropeptide is generated from a PG that is not associated with a lipoprotein.

121. The composition of any of claims 117-120, wherein said therapeutic PG muropeptide comprises at least one therapeutic muropeptide comprising a 5′ N-acetylglucosamine (NAG)-N-acetylmuramic acid (NAM) disaccharide muropeptide with an amino acid peptide attached to said NAM.

122. The composition of claim 117, wherein said therapeutic PG muropeptide are selected from the group consisting of: a muropeptides dimer, a muropeptides oligomer, and a combination of the same.
